(19) United States
(12) Patent Application Publication
(10) Pub. No.: US 2026/0294856 A1
(43) Pub. Date: Oct. 1, 2026

(54) METHODS FOR TREATING GASTROINTESTINAL INFLAMMATORY DISEASES USING ALPHA-KETOGLUTARATE (AKG) MODULATORS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Almudena Chavez, New York, NY (US); Scott Lowe, New York, NY (US)

(21) Appl. No.: 19/480,776

(22) PCT Filed: May 1, 2024
(86) PCT No.: PCT/US2024/027206

§ 371 Date: Oct. 31, 2025

Related U.S. Application Data
(60) Provisional application No. 63/463,741, filed on May 3, 2023.
(60) Provisional application No. 63/593,484, filed on Oct. 26, 2023.

(51) Int. Cl. A61K 31/194; A61K 31/19; A61K 31/198; A61K 31/4025; A61K 31/404; A61K 31/405; A61K 31/4245; A61K 31/436; A61K 31/4402; A61K 31/4545; A61K 31/4985; A61K 31/517; A61K 31/519; A61K 31/52; A61K 31/573; A61K 31/58; A61K 31/606; A61K 31/662; A61K 31/7068; A61P 1/04; C12N 15/113; C12Q 1/32; C12Q 1/6883

(52) U.S. Cl. CPC A61K 31/194; A61K 31/19; A61K 31/198; A61K 31/4025; A61K 31/404; A61K 31/405; A61K 31/4245; A61K 31/436; A61K 31/4402; A61K 31/4545; A61K 31/4985; A61K 31/517; A61K 31/519; A61K 31/52; A61K 31/573; A61K 31/58; A61K 31/606; A61K 31/662; A61K 31/7068; A61P 1/04; C12N 15/113; C12Q 1/32; C12Q 1/6883; C12N 2310/531; C12Q 2600/158

(57) ABSTRACT

The present disclosure provides methods and compositions for treating or preventing a gastrointestinal inflammatory disease in a subject in need thereof comprising administering to the subject a therapeutically effective amount of an agent that increases alpha-ketoglutarate (aKG) expression and/or activity.

### CROSS-REFERENCE TO RELATED APPLICATIONS

This application claims the benefit of and priority to U.S. Provisional Patent Application No. 63/463,741, filed May 3, 2023, and U.S. Provisional Patent Application No. 63/593,484, filed Oct. 26, 2023, the entire contents of which are incorporated herein by reference.

### GOVERNMENT SUPPORT

This invention was made with government support under CA254838 awarded by the National Institutes of Health. The government has certain rights in the invention.

### TECHNICAL FIELD

The present technology relates to methods and compositions for treating or preventing gastrointestinal inflammatory disease in a subject in need thereof comprising administering a therapeutically effective amount of an agent that increases alpha-ketoglutarate (αKG) expression and/or activity.

### BACKGROUND

The following description of the background of the present technology is provided simply as an aid in understanding the present technology and is not admitted to describe or constitute prior art to the present technology.

Cells in the mammalian intestine undergo a hierarchical process of differentiation leading to the lineage specification of various cell types that make up the intestinal crypt^{9,10}. Intestinal stem cells (ISCs), located at the bottom of the crypt, possess the ability to both self-renew and potentially differentiate into the distinct lineages found within the intestinal epithelium. In the bottom of the crypt, stemness is maintained by strict balance among Notch pathway, bone morphogenic protein (BMP) pathway, and Wnt and its agonist R-spondin (Rspo)¹¹⁻¹³. As ISCs undergo division, they migrate to the transit-amplifying cell compartment and give rise to progenitor cells¹⁴, with subsequent activation of lineage-specific transcriptional programs enabling these progenitors to fully differentiate into mature absorptive and secretory lineages. In the absorptive lineage, enterocytic progenitors are highly proliferative cells that must rapidly expand to generate the absorptive surface in the intestine (260-300 m² in humans)^{11,15,16}. In contrast, the differentiation towards the secretory lineage involves a more limited expansion that gives rise to smaller yet diverse pool of cells, including goblet, enteroendocrine, Paneth, and tuft cells. These specialized cells coordinate immune responses, secrete antimicrobial peptides, and produce mucus, all of which are essential to intestinal health^{9,10}. Tissue damage can disrupt the normal trajectory of intestinal differentiation, resulting in impaired maturation and decreased generation of secretory cells^{4,5,17}. This imbalance can contribute to the development of inflammatory bowel diseases, such as ulcerative colitis (UC) and Crohn's disease in humans, for which additional therapeutic options are needed^{4,5,17}.

Accordingly, there is an urgent need for compositions that effectively treat or prevent gastrointestinal inflammatory diseases such as UC and Crohn's disease in patients in need thereof.

### SUMMARY OF THE PRESENT TECHNOLOGY

In one aspect, the present disclosure provides a method for treating or preventing a gastrointestinal inflammatory disease in a subject in need thereof comprising administering to the subject a therapeutically effective amount of an agent that increases alpha-ketoglutarate (αKG) expression and/or activity. In another aspect, the present disclosure provides a method for selecting a subject suffering from a gastrointestinal inflammatory disease for treatment with an agent that increases alpha-ketoglutarate (αKG) activity comprising detecting OGDH mRNA and/or OGDH polypeptide levels in a biological sample obtained from the patient that are elevated relative to a predetermined threshold or a reference sample obtained from a healthy control subject; and administering to the subject an effective amount of an agent that increases αKG activity. Additionally or alternatively, in some embodiments, the expression levels of OGDH are detected via RT-PCR, Northern Blotting, RNA-Seq, microarray analysis, High-performance liquid chromatography (HPLC), mass spectrometry, immunohistochemistry (IHC), fluorescence in situ hybridization (FISH), Western Blotting, immunoprecipitation, flow cytometry, Immuno-electron microscopy, immunoelectrophoresis, enzyme-linked immunosorbent assays (ELISA), or multiplex ELISA antibody arrays.

In any and all embodiments of the methods disclosed herein, the gastrointestinal inflammatory disease is colitis or Chron's disease.

Additionally or alternatively, in some embodiments, the agent is αKG, glutamine, succinate, or dimethyl-succinate. In any of the preceding embodiments of the methods disclosed herein, the agent inhibits expression and/or activity of a component of oxoglutarate dehydrogenase (OGDH) complex. The components of OGDH complex include oxoglutarate dehydrogenase (OGDH), dihydrolipoyl succinyltransferase (DLST), or dihydrolipoyl dehydrogenase (DLD). In some embodiments, the agent is a small molecule, an inhibitory nucleic acid that specifically targets the component of OGDH complex, or a neutralizing antibody that specifically targets the component of OGDH complex. Examples of small molecule OGDH inhibitors include, but are not limited to succinyl phosphonate, (S)-2-[(2,6-dichlorobenzoyl)amino]succinic acid (AA6), KGD09, KGD02, and derivatives (e.g., azide-alkyne cyclized derivatives of KGD09, and KGD02) thereof. Examples of small molecule DLST inhibitors include, but are not limited to obatoclax mesylate, CP724714, LY317615 (enzastaurin), gemcitabine, BMS 708163 (Avagacestat), Lapatinib, or IPA3. Examples of small molecule DLD inhibitors include, but are not limited to carmustine, lomustine, 5-methoxyindole-2-carboxylic acid (MICA), valproic acid (VPA) derivatives such as valproyl-CoA and valproyl-dephospho-CoA. In some embodiments, the inhibitory nucleic acid that specifically targets the component of OGDH complex is a siRNA, a shRNA, an antisense oligonucleotide, or a sgRNA. The inhibitory nucleic acid that specifically targets the component of OGDH complex may be operably linked to a heterologous promoter, an inducible promoter, a constitutive promoter, a tissue-specific promoter, or an ubiquitous promoter. In some embodiments, the inhibitory nucleic acid that specifically targets the component of OGDH complex is selectively expressed in intestinal epithelial cells.

In any of the preceding embodiments, the agent is administered orally, topically, intranasally, systemically, intravenously, subcutaneously, intraperitoneally, intradermally, intraocularly, iontophoretically, transmucosally, or intramuscularly.

Additionally or alternatively, in some embodiments, the methods of the present technology further comprise separately, sequentially or simultaneously administering one or more additional therapeutic agents to the subject. Examples of additional therapeutic agents that are useful for treating or preventing gastrointestinal inflammatory diseases include, but are not limited to, aminosalicylates (e.g., sulfasalazine, mesalamine, olsalazine, balsalazide), corticosteroids (e.g., Prednisone, Prednisolone, Methylprednisolone, Budesonide), azathioprine, 6-mercaptopurine, cyclosporine, tacrolimus, ozanimod, tofacitinib, upadacitinib, etrasimod, adalimumab, golimumab, infliximab, ustekinumab and vedolizumab.

In any and all embodiments of the methods disclosed herein, administration of the agent that increases αKG activity results in an increased abundance of secretory intestinal cells in the subject. Additionally or alternatively, in some embodiments, administration of the agent does not alter biosynthetic function or viability of enterocytes in the subject.

In any and all embodiments of the methods disclosed herein, the agent that increases αKG activity is specifically targeted for delivery into intestinal epithelium using a nanocarrier. The nanocarrier may comprise an extracellular vesicle, a lipid-based nanoparticle, a microsphere, a liposome, an inorganic nanoparticle, a SPION, a SiNP, a SNEDD, or a polymeric nanoparticle. In some embodiments, the nanocarrier is functionalized with PEG, listeriolysin O (LLO), silica mesoporous microparticles, an antibody, a carbohydrate, or a ligand that binds a receptor that overexpressed in intestinal epithelium. Additionally or alternatively, in some embodiments, the agent that increases aKG activity further comprises a pH-dependent release coating or a delayed-release coating. In some embodiments, the pH-dependent release coating or the delayed-release coating comprises one or more of hyaluronic acid, alginate, Eudragit, polysaccharides, inulin, xanthan gum, locust bean gum, portulaca-derived polysaccharides, pectin, guar gum, carboxymethyl starch (CM), Layered double hydroxides (LDHs), calixarenes, and cholesteryl hemisuccinate (CHEMS).

In one aspect, the present disclosure provides a method for enhancing tissue regeneration in a subject that has received radiation therapy comprising administering to the subject a therapeutically effective amount of alpha-ketoglutarate (αKG) or glutamine. In some embodiments, the radiation therapy is external beam radiation therapy. The tissue regeneration may comprise regeneration of intestinal tissue or colon tissue.

Also disclosed herein are kits comprising at least one of alpha-ketoglutarate (αKG), glutamine, succinate (e.g., dimethyl-succinate), an OGDH inhibitor, a DLST inhibitor or a DLD inhibitor described herein and instructions for using the same to treat or prevent gastrointestinal inflammatory disease (e.g., colitis or Chron's disease) or enhance tissue regeneration in a subject that has received radiation therapy. In some embodiments, the OGDH inhibitor, DLST inhibitor or DLD inhibitor is a small molecule, an inhibitory nucleic acid that specifically targets the component of OGDH complex, or a neutralizing antibody that specifically targets the component of OGDH complex. Examples of small molecule OGDH inhibitors include, but are not limited to succinyl phosphonate, (S)-2-[(2,6-dichlorobenzoyl)amino]succinic acid (AA6), KGD09, KGD02, and derivatives (e.g., azide-alkyne cyclized derivatives of KGD09, and KGD02) thereof. Examples of small molecule DLST inhibitors include, but are not limited to obatoclax mesylate, CP724714, LY317615 (enzastaurin), gemcitabine, BMS 708163 (Avagacestat), Lapatinib, or IPA3. Examples of small molecule DLD inhibitors include, but are not limited to carmustine, lomustine, 5-methoxyindole-2-carboxylic acid (MICA), valproic acid (VPA) derivatives such as valproyl-CoA and valproyl-dephospho-CoA. In some embodiments, the inhibitory nucleic acid that specifically targets the component of OGDH complex is a siRNA, a shRNA, an antisense oligonucleotide, or a sgRNA. The inhibitory nucleic acid that specifically targets the component of OGDH complex may be operably linked to a heterologous promoter, an inducible promoter, a constitutive promoter, a tissue-specific promoter, or an ubiquitous promoter. In some embodiments, the inhibitory nucleic acid that specifically targets the component of OGDH complex is selectively expressed in intestinal epithelial cells.

### BRIEF DESCRIPTION OF THE DRAWINGS

FIGS. 1a-1m: Metabolic state correlates with lineage specification in the intestine. FIG. 1a: Expression of TCA cycle enzyme genes in different cell types in the intestinal tissue, derived from publicly available scRNA-seq data. The size of the dots represents the percentage of positive cells identified in the indicated lineage and the color represents the amount of expression. FIGS. 1b-1d: Single-molecule in situ fluorescence (smFISH) analysis depicting RNA expression of the indicated TCA cycle enzymes in intestinal tissue from C57Bl/6 mice. FIG. 1e: Scheme describing the reporter for intestinal stem cells introduced into the mice used in the subsequent experiments. FIG. 1f: Sorting strategy to isolate Paneth cells (GFP⁻, high FSC-A) and ISCs (GFP^{high}). FIG. 1g: Heatmap depicting qPCR values of various TCA cycle enzymes and lineage-specific markers in different intestinal stem cell populations. Paneth cells and ISCs were obtained as described in FIG. 1f, and absorptive lineage corresponds to the villus fraction obtained during crypt isolation process. FIG. 1h: Diagram describing the protocol to ex vivo culture and expand different intestinal progenitors. This strategy was used for all metabolic experiments described in the paper. FIG. 1i: Immunofluorescence for Ogdh in ISCs, progenitor cells, and fully mature lineages. FIG. 1j: Immunofluorescence and staining in progenitor cells and fully mature lineages showing lineage-specific markers (Ace2=enterocytes, Lysozyme (Lyz)=Paneth cells, Alcian blue PAS (ABP)=Goblet cells). FIG. 1k: PCA component analysis of the metabolites obtained by LC-MC/MS from the different intestinal population cultured ex vivo. FIG. 1l: Metabolite levels in different intestinal progenitors relative to ISCs with particular focus on bioenergetics and TCA cycle metabolites. FIG. 1m: Scheme depicting TCA cycle in absorptive lineage and the rewiring observed in the secretory lineage. (Abbreviations: ENR=EGF/Nogging/R-spondin; C=CHIR2099; V=Valproic acid; I=IWP2; D=DAPT; pAbs=absorptive progenitors; pSec1=goblet progenitors; pSec2=Paneth progenitors).

FIGS. 2a-2p: Dual role of Ogdh in lineage specification during tissue regeneration. FIG. 2a: Scheme depicting Ogdh depletion experiments in ISC-enriched organoids extracted from TRE-shRen^{Cag-rtTA3}, TRE-shOgdh^{Cag-rtTA3}, or wildtype mice. After ISC-enriched organoids were expanded in ENR-CV media, TRE-shRen^{Cag-tTA3} and TRE-shOgdh^{Cag-rTA3} were treated with doxycycline and wildtype organoids with DM-αKG, and simultaneously changed to ENR media to allow differentiation into all cell types in the intestine. FIG. 2b: Immunofluorescence and staining in ISC-enriched organoids from TRE-shRen^{Cag-rTA3}, TRE-shOgdh^{Cag-rt TA3}, and DM-αKG-treated organoids showing proliferation (Ki67) and cell death (Cl. Casp3) after 3 days of doxycycline or DM-αKG, and lineage specific markers (Ace2=enterocytes, lysozyme (Lyz)=Paneth cells, Alcian blue PAS (ABP)=goblet cells) after 6 days. FIG. 2c: Scheme depicting Ogdh depletion and αKG supplementation experiments in progenitor-enriched organoids. ISC-enriched organoids were expanded, differentiated, and then treated with either doxycycline (TRE-shRen^{Cag-rtTA3}, TRE-shOgdh^{Cag-rTA3} organoids) or DM-αKG (wildtype) and maintained. Organoids were maintained in the pertinent lineage-specific differentiation media until the end of the experiment to force differentiation into the intended cell lineage. FIG. 2d: Immunofluorescence in progenitor-enriched organoids from TRE-shRen^{Cag-rtTA3}, TRE-shOgdh^{Cag-rtTA3}, and αKG-treated organoids showing proliferation (Ki67) and cell death (Cl. Casp3) after 3 and 8 days in culture. FIG. 2e: Immunofluorescence in progenitor-enriched organoids from C57Bl/6 mice showing Tet expression and 5hmC in the different intestinal lineages. FIG. 2f: αKG and αKG:succinate ratio in ISCs and the different progenitors, measured by LC-MS/MS (See FIG. 1). FIG. 2g: Oxygen consumption rate (OCR) obtained through Seahorse experiments performed in organoids derived from TRE-shRen^{Cag-rtTA3} or TRE-shOgdh^{Cag-rTA3} mice. FIG. 2h: ATP production in TRE-shRen^{Cag-tTA3} or TRE-shOgdh^{Cag-rtTA3} mice measured by Seahorse analysis. FIGS. 2i-2j Metabolite levels in different intestinal progenitors relative to ISCs and in TRE-shOgdh^{Cag-rtTA3} organoids relative to TRE-shRen^{Cag-rtTA3} organoids, with particular focus on TCA cycle metabolites (FIG. 2i) and bioenergetics (FIG. 2j). FIG. 2k: Scheme depicting potential binding sites for Hnf4α in the Ogdh promoter and the position relative to the transcription start site (TSS). FIG. 2l: Immunofluorescence in progenitor-enriched organoids from C57Bl/6 mice showing Hnf4α expression in the different lineages. FIG. 2m: Hnf4α chromatin immunoprecipitation (ChIP) of intestinal isolated crypts from C57Bl/6 mice. qPCR was run using 3 different primer sets in the immunoprecipitated DNA for Hnf4α (positive binding) and Smad4 (part of the complex but lacks DNA-binding domains). FIG. 2n: Immunofluorescence analysis in tissue sections from C57Bl/6 mice showing Hnf4a expression and 5hmC-positive marks within the crypt compartment. FIG. 2o: Percentage of colocalization between 5hmC and Hnf4α in tissue sections obtained from FIG. 2l. FIG. 2p: 5hmC and Hnf4α expression according to crypt size in tissue sections obtained from FIG. 2l. Statistical analysis: t-test was used to assess the statistical significance of the results.

FIGS. 3a-3m: Role of Ogdh during intestinal homeostasis in vivo. FIG. 3a: Hematoxylin and eosin staining (H & E) and immunofluorescence analysis for GFP, Ogdh, BrdU, and Cleaved Caspase 3 (Cl. Casp 3) in intestinal sections at day 3 of doxycycline treatment. FIG. 3b: H & E and immunofluorescence staining for GFP, Cl. Casp 3, and Hnf4α in intestinal sections at day 6 of doxycycline treatment. FIGS. 3c-3d: Time course quantification of BrdU and Cl. Casp 3 in intestinal sections from TRE-shRen^{Cag-rtTA3} and TRE-shOgdh^{Cag-rtTA3} mice treated with doxycycline. FIG. 3e: H & E and immunofluorescence staining for Cl. Casp 3, BrdU, ß-catenin (Bcat), and Hnf4α in intestinal sections from mice injected with 600 mg/kg αKG and treated for 7 days. FIG. 3f: Alcian blue periodic acid Schiff (ABP) staining and immunofluorescence staining for lysozyme and Ace2 in intestinal sections from TRE-shRen^{Cag-rtTA3}, TRE-shOgdh^{Cag-rtTA3}, vehicle-treated, and DM-αKG-treated mice. FIG. 3g: Immunofluorescence staining for Hnf4α and 5hmC in intestinal sections from TRE-shOgdh^{Cag-rtTA3} and DM-αKG treated mice. FIG. 3h: 5hmC levels in intestinal tissue from TRE-shRen^{Cag-rtTA3}, TRE-shOgdh^{Cag-rtTA3}, vehicle-treated, and DM-αKG treated mice measured by ELISA using total tissue lysates. FIG. 3i: Experimental design scheme for RNA seq experiments in isolated intestinal crypts. FIG. 3j: Gene ontology (GO) analysis showing the most upregulated and downregulated pathways in TRE-shOgd^{Cag-rtTA3} vs TRE-shRen^{Cag-rtTA3} mice. FIG. 3k: Heatmap illustrating transcriptional changes associated with secretory lineage, absorptive lineage (zone 1 and zone 2), Notch signaling pathway, and proliferation in crypts isolated from TRE-shRen^{Cag-rtTA3}, TRE-shOgdh^{Cag-rtTA3}, vehicle-treated, and DM-αKG-treated mice. FIG. 3l: Venn diagrams depicting overlap in upregulated and downregulated genes between TRE-shOgdh^{Cag-rtTA3} and αKG-treated mice, along with GO analysis for the overlapping upregulated genes. FIG. 3m: Scheme illustrating the skewing towards differentiation into the secretory lineage following Ogdh depletion or αKG supplementation.

FIGS. 4a-4q: Metabolic interventions to treat ulcerative colitis. FIG. 4a: Immunofluorescence staining for Ogdh, Ki67, and 5hmC, and Alcian blue periodic acid Schiff (ABP) staining in tissue microarrays of human intestinal tissue with or without colitis. FIG. 4b: Immunofluorescence staining for Ogdh, Ki67, and 5hmC (Yellow in human samples, red in mouse tissue), and ABP staining in intestinal tissue from mice with or without DSS-induced colitis. FIG. 4c: Quantification of OGDH and ABP levels in human samples from normal, inflamed, ulcerative colitis (UC), and Crohn's mucosa. FIG. 4d: Time course quantification of OGDH and ABP levels in DSS-treated mice (R=recovery, 15 days). FIG. 4e: 5hmC levels quantified by ELISA in lysates from total colon tissue of mice treated with DSS. FIG. 4f: Experimental scheme illustrating the treatment for DSS-induced ulcerative colitis in mice with pulsatile OGDH inhibition. FIG. 4g: Body weight in the indicated conditions, presented as a percentage of initial body weight. FIG. 4h: Colon length for mice in the indicated conditions at day 9. Colon length is a readout of colonic inflammation and damage; a shorter length indicates a more inflamed and damaged colon. FIG. 4i: Hematoxylin and eosin (H & E) staining and ABP staining of intestinal sections from mice treated with DSS or DSS+pulsatile OGDH inhibition at day 9. FIG. 4j: Numbers of ulcers in the depicted conditions at day 9 of treatment. FIG. 4k: ABP quantification at day 9 in the indicated conditions. FIG. 4l: Experimental scheme illustrating the treatment for DSS-induced ulcerative colitis in mice treated with αKG. FIG. 4m: Body weight in the indicated conditions relative to the initial body weight. FIG. 4n: Colon length at day 9 in the indicated conditions. FIG. 4o: H & E and ABP staining of intestinal sections from mice treated with DSS or DSS+αKG at day 9. FIG. 4p: Numbers of ulcers in the depicted conditions at day 9 of treatment. FIG. 4q: ABP quantification at day 9 in the indicated conditions.

FIGS. 5a-5f: Characterization of Ogdh expression in the intestine. FIG. 5a: Principal component analysis (PCA) of publicly available scRNA-seq data showing different transcriptional signatures in various human intestinal and colonic cell populations. FIG. 5b: Immunofluorescence staining in intestinal tissues from Lgr5-EGFP mice, depicting GFP (ISCs), Ogdh, Ace2 (enterocytes), and Lyz (Paneth cells). FIG. 5c: Quantification of OGDH expression percentage in different intestinal lineages. FIGS. 5d-5f: Receiver operating characteristic (ROC) curve analysis demonstrating the specificity of OGDH as a marker for enterocytes (FIG. 5d), ISCs (FIG. 5e) and Paneth cells (FIG. 5f).

FIGS. 6a-6f: Mitochondrial activity in different intestinal lineages. FIG. 6a: Principal component analysis (PCA) of metabolites obtained through LC-MS/MS in ISCs, absorptive progenitors (pAbs), and secretory progenitors (pSec1 and pSec2). FIG. 6b: Hierarchical clustering dendrogram based on metabolites obtained by LC-MS/MS in different intestinal organoids enriched for various intestinal populations. FIG. 6c: Image of organoids growing in a Seahorse plate. These organoids were used for Seahorse analysis. FIG. 6d: Oxygen consumption rate (OCR) derived from Seahorse analysis in different progenitor lineages. FIG. 6e: Immunofluorescence staining in intestinal tissues from Lgr5-EGFP mice, showing GFP (ISCs), VDAC (mitochondrial marker), and β-catenin (membrane marker). FIG. 6f: Quantification of VDAC intensity in ISCs and Paneth cells.

FIGS. 7a-7g: Generation of an inducible Ogdh knock down mouse model. FIG. 7a: Schematic representation of the generation of the TRE-shOgdh^{Cag-rtTA3} knockdown mouse model using the “speedy-mouse technology” (See Materials and Methods). FIG. 7b: Pictures showing GFP induction in shOgdh.2081 and shOgdh.346 mouse embryonic stem cells (mESCs) after 48 hr with doxycycline. FIG. 7c: Ogdh expression measured by qPCR in shRenilla.713, shOgdh.2081, and shOgdh.346 mESCs after 48 hr on doxycycline treatment. FIG. 7d: Western blot analysis of mouse mESCs used for generating the mouse model, 48 hours after doxycycline treatment. FIG. 7e: Genotyping PCR results showing wild-type mice (248 bp) and knock-in mice (300 bp). FIG. 7f: Representative macroscopic bright-field images and GFP fluorescent images of different organs from TRE-shOgdh^{Cag-rtTA3} mice with or without doxycycline diet for 6 days. FIG. 7g: Immunofluorescence analysis of Ogdh in different tissues at day 3 of doxycycline treatment in TRE-shOgdh^{rtTA3} and TRE-shRenilla^{Cag-rtTA3} mice.

FIGS. 8a-8o: Role of Ogdh during intestinal homeostasis in vivo. FIG. 8a: Schematic representation of the experimental design in TRE-shOgdh^{Cag-rtTA3} mice, where Ogdh depletion is induced in adulthood. FIG. 8b: qPCR analysis showing Ogdh expression in intestinal lysates from TRE-shOgd^{Cag-rtTA3} and TRE-shRen^{Cag-rtTA3} mice after 3 days of doxycycline treatment. FIG. 8c: Kaplan-Meier survival curve comparing TRE-shOgdh^{Cag-rtTA3} and TRE-shRen^{Cag-rtTA3} mice during doxycycline treatment. FIG. 8d: Percentage of body weight compared to initial body weight in TRE-shOgdh^{Cag-rtTA3} and TRE-shRen^{Cag-rtTA3} mice. FIG. 8e: Weight of food found in the stomachs of TRE-shOgdh^{Cag-rtTA3} and TRE-shRen^{Cag-rtTA3} mice on the 8th day of doxycycline treatment. FIG. 8f: Schematic representation of the experimental design for DM-αKG injection in C57Bl/6 adult mice. FIG. 8g: Body weight compared to initial body weight in mice treated with vehicle or two different doses of DM-αKG. FIG. 8h: Kaplan-Meier survival curves of mice treated with vehicle or two different doses of DM-αKG. FIG. 8i: Single-molecule in situ fluorescence (smFISH) analysis depicting the RNA expression patterns of the indicated differentiation markers in intestinal tissue from TRE-shOgdh^{Cag-rtTA3} and TRE-shRen^{Cag-rtTA3} mice. FIG. 8j: Immunofluorescence staining for Lysozyme (Lyz), BrdU, and Æ-catenin in intestinal sections from vehicle-treated and DM-αKG-treated mice after a 2-hour pulse with BrdU. FIG. 8k: Immunofluorescence staining and quantification of Olfm4 levels in intestinal tissues from TRE-shOgdh^{Cag-rt TA3}, TRE-shRen^{Cag-rtTA3}, vehicle-treated, and DM-αKG-treated mice at day 4 and day 7 of the treatment, respectively. FIG. 8l: Gene ontology (GO) analysis showing the most upregulated pathways in intestinal crypts from mice treated with DM-αKG vs vehicle. FIG. 8m: Heatmap analysis showing Wnt pathway gene expression profiles in crypts extracted from TRE-shOgdh^{Cag-rtTA3}, TRE-shRen^{Cag-rtTA3} vehicle-treated, and DM-αKG-treated mice at day 3 of treatment. Each lane represents one mouse. FIG. 8n: Heatmap analysis showing gene expression profiles of transcription factors involved in lineage specification in crypts extracted from TRE-shOgdh^{Cag-rtTA3}, TRE-shRen^{Cag-rtTA3} vehicle-treated, and DM-αKG-treated mice at day 3 of treatment. FIG. 8o: Plot depicting predicted transcription factors commonly upregulated or downregulated in TRE-shOgdh^{Cag-rtTA3} vs. DM-αKG-treated mice. Red dots indicate upregulation, blue dots indicate downregulation.

FIGS. 9a-9h: Metabolic interventions to treat ulcerative colitis. FIG. 9a: Expression of TCA cycle enzymes in colon tissue from publicly available single-cell RNA sequencing (scRNA-seq) data, comparing healthy, non-inflamed tissue from ulcerative colitis patients, and inflamed ulcerative colitis conditions. Each row represents a condition, each column represents the expression of a specific TCA cycle enzyme across conditions. The size of the dots represents the percentage of positive cells identified in that lineage, and the color indicates the level of expression according to that column. FIG. 9b: Correlation analysis between Ki67, Ogdh, and ABP expression in ulcerative colitis, Crohn's disease, chronic inflammation, and normal mucosa, obtained from the tissue microarrays shown in FIG. 4a. The bottom row shows only individuals with normal mucosa or chronic inflammation. FIG. 9c: Kaplan-Meier survival curve comparing constant doxycycline treatment with pulsatile inhibition (3 days ON/4 days OFF/weekly) in TRE-shOgd^{Cag-rtTA3} mice. FIG. 9d: Representative images showing colon length of TRE-shOgdh^{Cag-rtTA3} mice on day 9 under different conditions. FIG. 9e: Representative images showing colon length of αKG-treated mice on day 9 under different conditions. FIG. 9f: Representative flow cytometric immunophenotyping of myeloid populations in immune cells isolated from the entire colon of mice given the indicated treatments. FIG. 9g: Quantification of macrophages, monocytes, granulocytes, and macrophage status from the immunophenotyping analysis. FIG. 9h: Immunofluorescence staining for Cd8, Cd4, and Ki67 in mouse tissue from DSS and DSS+αKG-treated mice.

FIGS. 10a-10i: Metabolic intervention to prevent ulcerative colitis. FIG. 10a: Schematic of the DM-αKG treatment as preventative for DSS-induced ulcerative colitis. FIG. 10b: Body weight in the indicated conditions relative to the initial body weight. FIG. 10c: Representative images showing colon length of αKG-treated mice on day 9 under the indicated conditions. FIG. 10d: Colon length at day 9 in the indicated conditions. FIG. 10e: Hematoxylin and eosin (H & E) staining and Alcian blue periodic acid Schiff (ABP) staining in intestinal sections from mice treated with DSS or DSS+αKG at day 9. FIG. 10f: Numbers of ulcers in the depicted conditions at day 9 of treatment. FIG. 10g: ABP quantification at day 9 in the indicated conditions. FIG. 10h: Representative flow cytometric immunophenotyping of myeloid populations in immune cells isolated from the entire colon of mice given the indicated treatments. FIG. 10i: Quantification of macrophages, monocytes, granulocytes, and macrophage status from the immunophenotyping analysis.

FIGS. 11a-11g: Increases in αKG levels is sufficient to tailor stem cell differentiation into secretory lineage. FIG. 11a: Scheme depicting Ogdh depletion experiments in ISC-enriched organoids extracted from TRE-shRen^{Cag-rtTA3}, TRE-shOgdh^{Cag-rtTA3} or wildtype mice. After ISC-enriched organoids were expanded in ENR-CV media, TRE-shRen^{Cag-rtTA3} and TRE-shOgdh^{Cag-rtTA3} were treated with doxycycline and wildtype organoids with DM-αKG, and simultaneously changed to ENR media to allow differentiation into all cell types in the intestine. FIG. 11b: Immunofluorescence and staining in ISC-enriched organoids from TRE-shRen^{Cag-rtTA3}, TRE-shOgdh^{Cag-rtTA3}, and DM-aKG-treated organoids showing proliferation (Ki67) and cell death (Cl. Casp3) after 3 days of doxycycline or DM-αKG, and lineage specific markers (Ace2=enterocytes, lysozyme (Lyz)=Paneth cells, Alcian blue PAS (ABP)=goblet cells) after 6 days. FIG. 11c: Immunofluorescence in progenitor-enriched organoids from C57Bl/6 mice showing Tet expression and 5hmC in the different intestinal lineages. FIG. 11d: αKG and αKG:succinate ratio in ISCs and the different progenitors, measured by LC-MS/MS. FIG. 11e: Immunofluorescence analysis in tissue sections from C57Bl/6 mice showing Hnf4a expression and 5hmC-positive marks within the crypt compartment. FIG. 11f: Percentage of colocalization between 5hmC and Hnf4α in tissue sections obtained from FIG. 11e. FIG. 11g: 5hmC and Hnf4α expression according to crypt size in tissue sections obtained from FIG. 11e.

FIGS. 12a-12f: Lineage-dependent heterogeneity for Ogdh. FIG. 12a: Scheme depicting Ogdh depletion and αKG supplementation experiments in progenitor-enriched organoids. ISC-enriched organoids were expanded, differentiated, and then treated with either doxycycline (TRE-shRen^{Cag-rtTA3}, TRE-shOgdh^{Cag-rtTA3} organoids) or DM-αKG (wildtype) and maintained. Organoids were maintained in the pertinent lineage-specific differentiation media until the end of the experiment to force differentiation into the intended cell lineage. FIG. 12b: Immunofluorescence in progenitor-enriched organoids from TRE-shRen^{Cag-rtTA3}, TRE-shOgdh^{Cag-rtTA3}, and αKG-treated organoids showing proliferation (Ki67) and cell death (Cl. Casp3) after 3 and 8 days in culture. FIG. 12c-12d: Metabolite levels in different intestinal progenitors relative to ISCs and in TRE-shOgdh^{Cag-rtTA3} organoids relative to TRE-shRen^{Cag-rtTA3} organoids, with particular focus on TCA cycle metabolites (FIG. 12c) and bioenergetics (FIG. 12d). FIG. 12e: Oxygen consumption rate (OCR) obtained through Seahorse experiments performed in organoids derived from TRE-shRen^{Cag-rtTA3} or TRE-shOgdh^{Cag-rtTA3} mice. FIG. 12f: ATP production in TRE-shRen^{Cag-rtTA3} or TRE-shOgd^{Cag-rtTA3} mice measured by Seahorse analysis.

FIGS. 13a-13c: Regulation of Ogdh expression. FIG. 13a: Scheme depicting potential binding sites for Hnf4α in the Ogdh promoter and the position relative to the transcription start site (TSS). FIG. 13b: Immunofluorescence in progenitor-enriched organoids from C57Bl/6 mice showing Hnf4α expression in the different lineages. FIG. 13c: Hnf4α chromatin immunoprecipitation (ChTP) of intestinal isolated crypts from C57Bl/6 mice. qPCR was run using 3 different primer sets in the immunoprecipitated DNA for Hnf4α (positive binding) and Smad4 (part of the complex but lacks DNA-binding domains).

FIGS. 14a-14f: Hnf4 modulates Ogdh expression and maintain enterocytic lineage. FIG. 14a: The inducible vector design consists of a backbone plasmid containing regulatory elements for conditional expression. In this case, a tetracycline-responsive promoter (TRE) or a doxycycline-inducible promoter (Tet-On). Integrated within the vector are two hairpin RNA (shRNA) sequences targeting Hnf4α and Hnf4g and a reporter protein (mCherry) that is expressed upon doxycycline treatment. FIG. 14b: Diagram illustrating the experimental strategy for establishing stable organoid cultures harboring the construct and achieving concurrent Hnf4 downregulation in intestinal stem cell (ISC)-enriched organoids. FIG. 14c: mCherry fluorescence observed in organoids 48 hours post-Dox treatment. FIGS. 14d-14f: qPCR results displaying mRNA levels of Hnf4α and Hnf4g (FIG. 14d), Ogdh (FIG. 14e), Math1, and Defa1 (FIG. 14f) in polyclonal populations following dual Hnf4 downregulation for 48 hours.

FIGS. 15a-15e: Hnf4 modulates Ogdh expression and maintain enterocytic lineage. FIG. 15a: Schematic representation of the reporter vector design for assessing Ogdh promoter activity. The vector design features the incorporation of wild-type (wt) and mutated Hnf4 transcription factor binding sites within the promoter region of the Ogdh gene. Green fluorescent protein (GFP) expression is controlled by this modified Ogdh promoter, allowing for the visualization of Ogdh promoter activity. Additionally, a constitutive promoter from the thymidine kinase (pTK) gene drives the expression of Luciferase, serving as an internal control for electroporation efficiency. FIG. 15b: Diagram illustrating the experimental strategy visualization of Ogdh promoter activity during ISCs differentiation in organoids. FIG. 15c: Representative image depicting TurboGFP expression in the absorptive lineage under the control of both Wt and mutated Ogdh promoters 48 hr post induction of differentiation. FIG. 15d: GFP/Luciferase ratio in the absorptive lineage under the control of both Wt and mutated Ogdh promoters 48 hr post induction of differentiation. FIG. 15e: Time course depicting GFP/Luciferase ratio in the absorptive lineage under the control of both Wt and mutated Ogdh promoters at indicated timepoints post transfection in a murine colorectal cancer cell line.

FIGS. 16a-16d: Ogdh depletion specifically in the intestinal epithelium impairs enterocytic formation and skews differentiation towards secretory lineage. FIG. 16a: Representative macroscopic bright-field images and GFP fluorescent images of different organs from TRE-shOgdh^{Cag-rtTA3} and TRE-shOgdh^{Villin-rtTA3} mice with doxycycline diet for 6 days. FIG. 16b: Hematoxylin and eosin staining (H & E) and immunofluorescence analysis for GFP, Ogdh and Ki67 in TRE-shRen^{Villin-rtTA3} and TRE-shOgdh^{Villin-rtTA3} mice with doxycycline. FIG. 16c: Immunofluorescence analysis for lysozyme (Paneth cells) and Alcian Blue PAS staining (Goblet cells) in TRE-shRen^{Villin-rtTA3} and TRE-shOgdh^{Villin-rtTA3} mice with doxycycline. FIG. 16d: Immunofluorescence analysis for 5hmC in TRE-shRen^{Villin-rtTA3} and TRE-shOgdh^{Villin-rtTA3} mice on doxycycline treatment.

FIGS. 17a-17c: Ogdh depletion in the colonic epithelium during colitis enhances tissue regeneration. FIG. 17a: Representative images showing colon length of TRE-shRen^{Villin-rtTA3} and TRE-shOgdh^{Villin-rtTA3} mice on day 9 during colitis onset. FIG. 17b: Body weight in the indicated conditions, presented as a percentage of initial body weight. FIG. 17c: Colon length for mice in the indicated conditions at 984 day 9. Colon length is a readout of colonic inflammation and damage; a shorter length indicates a more inflamed and damaged colon.

FIGS. 18a-18d: αKG is also effective in an autoimmune model of colitis. FIG. 18a: Schematic representing the adoptive transfer of CD4+ T cells from C57BL/6 mice to Rag2 knockout (KO) mice, along with the sorting process. FIG. 18b: Body weight in the indicated conditions, presented as a percentage of initial body weight. FIG. 18c: Colon length for mice in the indicated conditions at 8 weeks post Cd4 transfer. FIG. 18d: Hematoxylin and eosin (H & E) staining of colonic sections from Rag2 KO mice treated with or without αKG treatment at 8 weeks after CD4 T cell transfer.

FIGS. 19a-19e: Characterization and tracing of secretory progenitors in steady-state conditions and during the regenerative phase following injury. FIG. 19a: Hyperplex immunofluorescence analysis on DSS-treated mice in the prevention setting to evaluate the expression of various markers, including Ogdh, Vimentin, αSMA, Atoh1 (secretory progenitors), Ephb2 (ISCs), and Muc2 (mature secretory cells), both with and without αKG supplementation. FIGS. 19b-19d: Schematic representation illustrating the labeling and tracing of secretory progenitors under steady-state conditions (FIG. 19b) and during the regenerative phase following injury (FIG. 19c). This was achieved using the Atoh1-CreERT2 mouse line crossed with the RIK allele (LSL-Katushka) and subsequently analyzed via immunofluorescence analysis (FIG. 19e).

FIG. 20: αKG supplementation enhances tissue regeneration following abdominal radiation treatment. Hematoxylin and eosin (H & E) staining depicting intestinal and colonic sections from C57B16 mice abdominally irradiated with 14 Gy, treated either with or without αKG, at the one-week post-irradiation time point.

FIG. 21: Glutamine supplementation (a precursor for αKG) helps tissue healing during colitis. Hematoxylin and eosin (H & E) staining illustrating colonic sections from C57B16 mice treated with DSS, with or without glutamine, in both preventive and therapeutic contexts, on day 9.

FIG. 22: Succinate supplementation rescues enterocytic cell death. Hematoxylin and eosin staining (H & E) and ABP staining (Goblet cells) and immunofluorescence analysis for Lysozyme (Paneth cells), GFP, Ogdh, Ace2 (mature enterocytes), Hnf4a, CC3 (apoptotic cells) and Ki67 in TRE-shRen^{Cag-rtTA3} and TRE-shOgdh^{Cag-rtTA3} mice on doxycycline treated with or without DM-succinate.

FIGS. 23a-23d: Existence of mitochondrial heterogeneity across different lineages in the intestine. FIG. 23a: Immunofluorescence analysis for Lysozyme (Paneth cells), VDAC (mitochondrial load), GFP in Lgr5-GFP mice and R catenin in intestinal epithelium. FIG. 23b: Quantification of VDAC expression in the different lineages in the intestinal epithelium. FIGS. 23c-23d: Detailed assessment of mitochondrial structure in the different lineages using electron microscopy. Quantification of mitochondrial parameters including size, shape, and density in ISCs, absorptive and secretory lineages.

FIGS. 24a-24d: TCA cycle rewiring following Ogdh depletion. FIGS. 24a-24d: Carbon tracing experiments conducted using ¹³C6 glucose and ¹³C5 glutamine isotope tracers. Upon Ogdh depletion, reductive carboxylation is observed (FIG. 24b), along with TCA cycle blockage (FIG. 24c) and increased activity of pyruvate carboxylase (FIG. 24d) to compensate for the deficiency of oxaloacetate resulting from the TCA cycle blockade.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

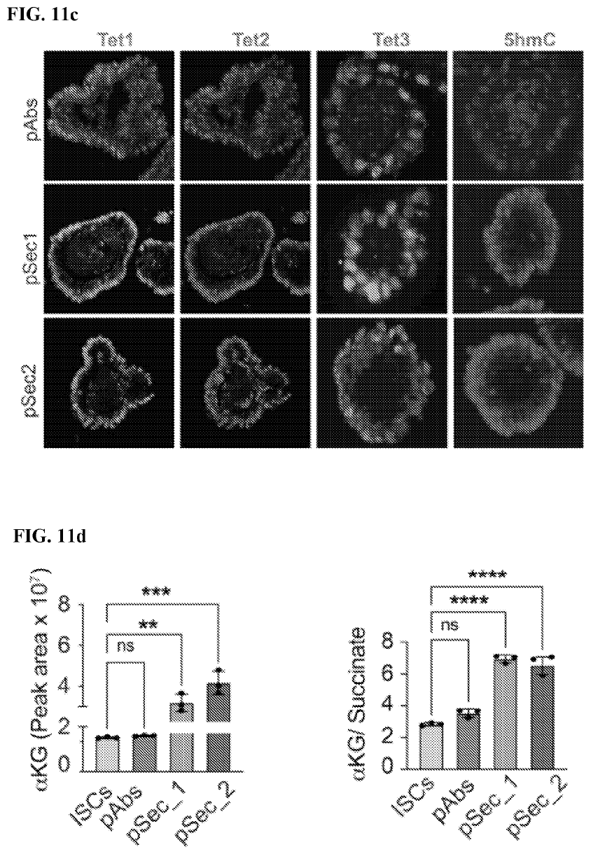

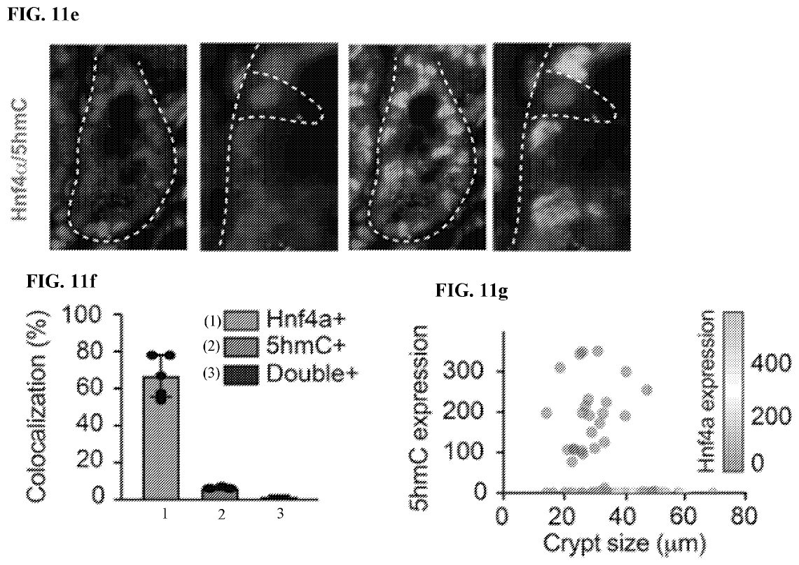

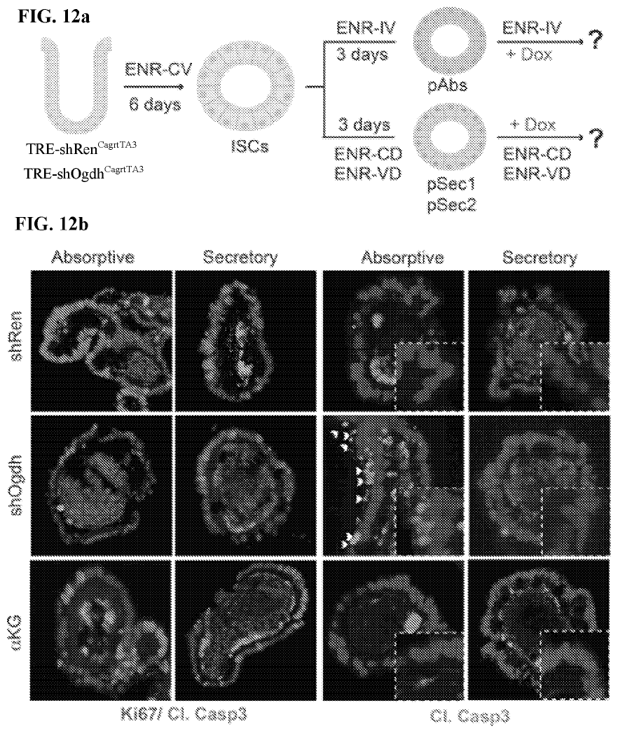

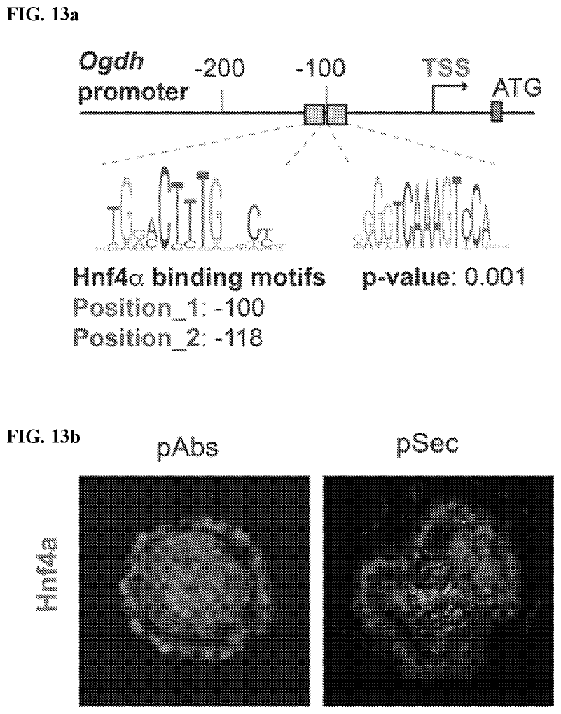

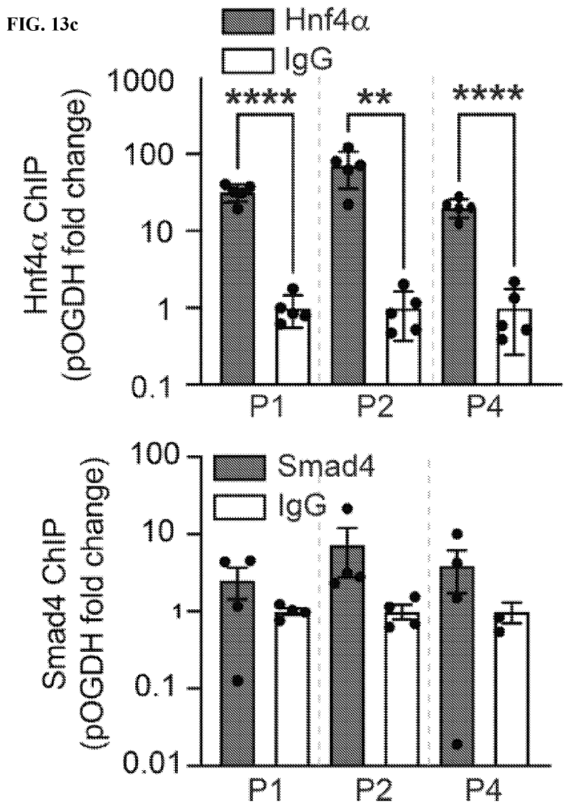

Addition of αKG or OGDH complex inhibition provides a therapeutic strategy to direct stem cell differentiation specifically into secretory cells, with implications for conditions like CRC tumors and colitis. This mechanism appears to hinge on increased activity of αKG-dependent dioxygenases and alterations in methylation levels within specific genes responsible for secretory lineage establishment (FIG. 11). Predicting which cells are Ogdh-dependent can be based on Ogdh expression at mRNA and protein level in the tissue, making Ogdh a potential biomarker for identifying responsive cells (FIG. 13). We also identified Hnf4α as a potential regulatory factor influencing Ogdh expression in a lineage-dependent manner.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, intrathecally, parenterally (intravenously, intramuscularly, intraperitoneally, or subcutaneously), rectally, intrathecally, intraocularly, intradermally, transmucosally, iontophoretically, or topically. Administration includes self-administration and the administration by another.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

The term “DLD inhibitor” as used herein refers to an agent that inhibits the expression and/or activity of dihydrolipoamide dehydrogenase (DLD), the E3 component of the αKG dehydrogenase complex. Examples of DLD biological activity includes, but is not limited to, an enzymatic activity, a substrate binding activity, homo- or hereto-dimerization activity and/or binding activity to a cellular structure. The DLD inhibitors of the present disclosure inhibit at least one biological activity of DLD.

The term “DLST inhibitor” as used herein refers to an agent that inhibits the expression and/or activity of dihydrolipoamide S-succinyltransferase (DLST), the E2 component of the αKG dehydrogenase complex. Examples of DLST biological activity includes, but is not limited to, an enzymatic activity, a substrate binding activity, homo- or hereto-dimerization activity and/or binding activity to a cellular structure. The DLST inhibitors of the present disclosure inhibit at least one biological activity of DLST.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “heterologous nucleic acid molecule or polypeptide” refers to a nucleic acid molecule (e.g., a cDNA, DNA or RNA molecule) or polypeptide that is either not normally expressed or is expressed at an aberrant level in a cell or sample obtained from a cell. This nucleic acid can be from another organism, or it can be, for example, an mRNA molecule that is not normally expressed in a cell or sample.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al., 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

The term “OGDH inhibitor” as used herein refers to an agent that inhibits the expression and/or activity of alpha-ketoglutarate dehydrogenase, which is also known as 2-oxoglutarate dehydrogenase (OGDH). Examples of OGDH biological activity includes, but is not limited to, an enzymatic activity, a substrate binding activity, homo- or hereto-dimerization activity and/or binding activity to a cellular structure. The OGDH inhibitors of the present disclosure inhibit at least one biological activity of OGDH.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, “prevention” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “specific” as used herein in reference to an oligonucleotide means that the nucleotide sequence of the oligonucleotide has at least 12 bases of sequence identity with a portion of a target nucleic acid when the oligonucleotide and the target nucleic acid are aligned. An oligonucleotide that is specific for a target nucleic acid is one that, under the stringent hybridization or washing conditions, is capable of hybridizing to the target nucleic acid of interest and not substantially hybridizing to nucleic acids which are not of interest. Higher levels of sequence identity are desirable and include at least 75%, at least 80%, at least 85%, at least 90%, at least 95% or at least 98% sequence identity.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “subject”, “patient”, or “individual” can be an individual organism, a vertebrate, a mammal, or a human. In some embodiments, the subject, patient or individual is a human.

As used herein, the terms “target sequence” and “target nucleic acid sequence” refer to a specific nucleic acid sequence to be modulated (e.g., inhibited or downregulated).

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Succinate, ODGH Inhibitors, DLST Inhibitors, or DLD Inhibitors of the Present Technology

Succinic acid is a dicarboxylic acid with the chemical formula (CH₂)₂(COOH)₂. In living organisms, succinic acid takes the form of an anion, succinate, which has multiple biological roles as a metabolic intermediate being converted into fumarate by the enzyme succinate dehydrogenase in complex 2 of the electron transport chain which is involved in making ATP, and as a signaling molecule reflecting the cellular metabolic state.

The present disclosure provides therapeutic agents that inhibit the activity or expression of OGDH, DLST, or DLD. In some embodiments, the OGDH inhibitor, the DLST inhibitor, or the DLD inhibitor is a small molecule, an inhibitory nucleic acid (e.g., siRNA, antisense nucleic acid, shRNA, sgRNA, ribozymes), or an antibody (e.g., a neutralizing antibody). Additionally or alternatively, in some embodiments, the OGDH inhibitor, the DLST inhibitor, or the DLD inhibitor is a selective inhibitor or non-selective inhibitor.

Examples of small molecule OGDH inhibitors include, but are not limited to succinyl phosphonate, (S)-2-[(2,6-dichlorobenzoyl)amino]succinic acid (AA6), KGD09, KGD02, and derivatives (e.g., azide-alkyne cyclized derivatives of KGD09, and KGD02) thereof. In some embodiments, the azide-alkyne cyclized derivatives of KGD09, and KGD02 would comprise at least one of the core moieties:

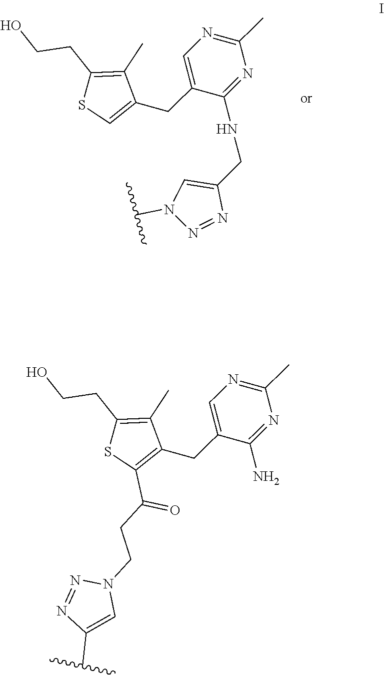 [SMILES: *n1cc(CNc2nc(C)ncc2Cc2csc(CCO)c2C)nn1 ; *c1cn(CCC(=O)c2sc(CCO)c(C)c2Cc2cnc(C)nc2N)nn1]

Examples of small molecule DLST inhibitors include, but are not limited to obatoclax mesylate, CP724714, LY317615 (enzastaurin), gemcitabine, BMS 708163 (Avagacestat), Lapatinib, or IPA3.

Examples of small molecule DLD inhibitors include, but are not limited to carmustine, lomustine, 5-methoxyindole-2-carboxylic acid (MICA), valproic acid (VPA) derivatives such as valproyl-CoA and valproyl-dephospho-CoA.

Exemplary mRNA sequences of OGDH are provided below, represented by SEQ ID NOs: 4-7:

NM_002541.4 Homo sapiens oxoglutarate dehydrogenase (OGDH), transcript
variant 1, mRNA
(SEQ ID NO: 4)
ATTCGGGTGGAGCTGAGCCGGAGACAGGCAGTTGTGAAAAACTTCAGGACAAAAATGTTTCATTTAAGGA
CTTGTGCTGCTAAGTTGAGGCCATTGACGGCTTCCCAGACTGTTAAGACATTTTCACAAAACAGACCAGC
AGCAGCTAGGACATTTCAACAGATTCGGTGCTATTCTGCACCTGTTGCTGCTGAGCCCTTTCTCAGTGGG
ACTAGTTCGAACTATGTGGAGGAGATGTACTGTGCTTGGCTGGAAAACCCCAAAAGTGTACATAAGTCAT
GGGACATTTTTTTTCGCAACACGAATGCCGGAGCCCCACCGGGCACTGCCTACCAGAGTCCCCTTCCCCT
GAGCCGAGGCTCCCTGGCTGCTGTGGCCCATGCACAGTCCCTGGTAGAAGCACAGCCCAACGTGGACAAG
CTCGTGGAGGACCACCTGGCAGTGCAGTCGCTCATCAGGGCATATCAGATACGAGGGCACCATGTAGCAC
AGCTGGACCCCCTGGGGATTTTGGATGCTGATCTGGACTCCTCCGTGCCCGCTGACATTATCTCATCCAC
AGACAAACTTGGGTTCTATGGCCTGGATGAGTCTGACCTCGACAAGGTCTTCCACTTGCCCACCACCACT
TTCATCGGGGGACAGGAATCAGCACTTCCTCTGCGGGAGATCATCCGTCGGCTGGAGATGGCCTACTGCC
AGCATATTGGGGTGGAGTTCATGTTCATCAATGACCTGGAGCAGTGCCAGTGGATCCGGCAGAAGTTTGA
GACCCCTGGGATCATGCAGTTCACAAATGAGGAGAAACGGACCCTGCTGGCCAGGCTTGTGCGGTCCACC
AGGTTTGAGGAGTTCCTACAGCGGAAGTGGTCCTCTGAGAAGCGCTTTGGTCTAGAAGGCTGCGAGGTAC
TGATCCCTGCCCTCAAGACCATCATTGACAAGTCTAGTGAGAATGGCGTGGACTACGTGATCATGGGCAT
GCCACACAGAGGGCGGCTGAACGTGCTTGCAAATGTCATCAGGAAGGAGCTGGAACAGATCTTCTGTCAA
TTCGATTCAAAGCTGGAGGCAGCTGATGAGGGCTCCGGAGATGTGAAGTACCACCTGGGCATGTATCACC
GCAGGATCAATCGTGTCACCGACAGGAACATTACCTTGTCCTTGGTGGCCAACCCTTCCCACCTTGAGGC
CGCTGACCCCGTGGTGATGGGCAAGACCAAAGCCGAACAGTTTTACTGTGGCGACACTGAAGGGAAAAAG
GTCATGTCCATCCTGTTGCATGGGGATGCTGCATTTGCTGGCCAGGGCATTGTGTACGAGACCTTCCACC
TCAGCGACCTGCCATCCTACACAACTCATGGCACCGTGCACGTGGTCGTCAACAACCAGATCGGCTTCAC
CACCGACCCTCGGATGGCCCGCTCCTCCCCCTACCCCACTGACGTGGCCCGAGTGGTGAATGCCCCCATT
TTCCACGTGAACTCAGATGACCCCGAGGCTGTCATGTACGTGTGCAAAGTGGCGGCCGAGTGGAGGAGCA
CCTTCCACAAGGACGTGGTTGTCGATTTGGTGTGTTACCGGCGCAACGGCCACAACGAGATGGATGAGCC
CATGTTCACGCAGCCGCTCATGTACAAGCAGATCCGCAAGCAGAAGCCTGTGTTACAGAAGTACGCTGAG
CTGCTGGTGTCGCAGGGTGTGGTCAACCAGCCTGAGTATGAGGAGGAAATTTCCAAGTATGATAAGATCT
GTGAGGAAGCTTTTGCCAGATCTAAAGATGAGAAGATCTTGCACATTAAGCACTGGCTGGACTCTCCCTG
GCCTGGCTTCTTCACCCTGGACGGGCAGCCCAGGAGCATGTCCTGCCCCTCCACGGGTCTGACGGAGGAT
ATTCTGACACACATCGGGAATGTGGCTAGTTCTGTGCCTGTGGAAAACTTTACTATTCATGGAGGGCTGA
GCCGGATCTTGAAGACTCGTGGGGAAATGGTGAAGAACCGGACTGTGGACTGGGCTCTAGCGGAGTACAT
GGCGTTTGGCTCGCTCCTGAAGGAGGGCATCCACATTCGGCTGAGCGGCCAGGACGTGGAGCGGGGCACA
TTCAGCCACCGCCACCATGTGCTCCATGACCAGAATGTGGACAAGAGAACCTGCATCCCCATGAACCATC
TCTGGCCCAATCAGGCCCCCTATACTGTGTGCAACAGCTCACTGTCTGAGTACGGCGTGCTGGGCTTTGA
GCTGGGCTTCGCCATGGCCAGTCCTAATGCCCTGGTCCTCTGGGAAGCCCAATTTGGTGACTTCCACAAC
ACGGCCCAGTGTATCATCGACCAGTTCATCTGCCCGGGACAAGCCAAGTGGGTGCGGCAGAATGGCATCG
TGTTGCTGCTGCCCCATGGCATGGAGGGCATGGGTCCAGAACATTCCTCCGCCCGCCCAGAGCGGTTCTT
GCAGATGTGCAACGATGACCCAGATGTCCTGCCAGACCTTAAAGAAGCCAACTTCGACATCAATCAGCTA
TATGACTGCAATTGGGTTGTTGTCAACTGCTCCACTCCTGGCAACTTCTTCCACGTGCTACGACGCCAGA
TCCTGCTGCCATTCCGGAAGCCGTTAATTATCTTCACCCCCAAATCCCTGTTGCGCCACCCCGAGGCCAG
ATCCAGCTTTGATGAGATGCTTCCAGGAACCCACTTCCAGCGGGTGATCCCAGAAGATGGCCCTGCAGCT
CAGAACCCAGAAAATGTCAAAAGGCTTCTCTTCTGCACCGGCAAAGTGTATTATGACCTCACCCGGGAGC
GCAAAGCACGCGACATGGTGGGGCAGGTGGCCATCACAAGGATTGAGCAGCTGTCGCCATTCCCCTTTGA
CCTCCTGCTGAAGGAGGTGCAGAAGTACCCCAATGCTGAGCTGGCCTGGTGCCAGGAGGAGCACAAGAAC
CAAGGCTACTATGACTACGTGAAGCCAAGACTTCGGACCACCATCAGCCGCGCCAAGCCCGTCTGGTATG
CCGGCCGGGACCCAGCGGCTGCTCCAGCCACCGGCAACAAGAAGACCCACCTGACGGAGCTGCAGCGCCT
CCTGGACACGGCCTTCGACCTGGACGTCTTCAAGAACTTCTCGTAGATGCTGCCTAGGGTTGCTTGGGCC
ACTGCCCTCTCCACACCCATGACTGCCCCTTGCTTCTCAACTAAAGAATAGTGCCTCAGCGCTGCCCACA
CCACCGCCCTCCTCGCTGTGCCACCACCCCTCCCTCTGCTCTCATAGGAGTTAGGCTGTCGTCCCCCTCC
AGTGCTTGGCTGCCCCACAGGCCACACGCTGCCCAGGCTCTGCTGACTTCTGAGCAGTTTTCCAGGAGGC
CGGGGGGAGCAGGAGGAGGAAAGGTAGCCCCCGAGGGATGTCCTTGGGGAGGGGTCAGCTCTGGCCACAA
TCCTCCCCACCAGTCTCACCCACTAGGATAGGAACTGGGCCTTGTGTGCTGGCTTCCGCTGTCACCCAGC
AAGGCACAGGCTCCTGTATTTGAGACTAGGATAGCTTCATCTTGAGCCTGAGCCTTAGAATCTGTAGAGG
AGCCTGGAGTCGGATCTAGCCATGGCTGGCAGAGGTTTCTAGGGTGGGCCCCAGCCGTGGCGTGAACTGA
GGATGACCCGGGGCAGCTGGCAGGAGAGAGCCTTGGCCTGACCTGGCACAGAAAGGGCAGCTTCAGTCTC
TGCAGTGTCCATTATCTGCTGTTCCTTCGAGGGTTCCAGGCTGTGTGTGGGGCCCAAGCATGCCCCACCC
ACCCCTCCTGGGCCCAGGCAGCACCTGGAGCCCACAGAGTCTGTGTGTAGCCAGGAAGCCCCGCTCAGGT
AGCCACCACCGGGGCACTGGCTGCTCTGTCTTGGTCCTGTTAACCCTCCACCTCCTCTCTTGGACTCCCT
CCCCACCCCAACCACTCTTTCTTTCTCCTTTAACCCAATGGAGACTTTCTGATGCATCGTTTTCTTTGCT
GTGCCAAAGCAGGTCAGAAGAGGGAGAGGAGGGGCTGGGGGTGAGGGGCCAGGCCATGGCCAAGGGGCCA
GCTGCCCCTCATTTATCACTCTGACCTTCACAGGGACAGATCTGATTTATTTATTTTGGTTAAAAAAAAA
AAAAAGGAACAGAAACAACTTTGCATTGCATTGGCTTGACCCATAAACTAAGTTATATCCGTG
NM_001003941.3 Homo sapiens oxoglutarate dehydrogenase (OGDH),
transcript variant 2, mRNA
(SEQ ID NO: 5)
ATTCGGGTGGAGCTGAGCCGGAGACAGGCAGTTGTGAAAAACTTCAGGACAAAAATGTTTCATTTAAGGA
CTTGTGCTGCTAAGTTGAGGCCATTGACGGCTTCCCAGACTGTTAAGACATTTTCACAAAACAGACCAGC
AGCAGCTAGGACATTTCAACAGATTCGGTGCTATTCTGCACCTGTTGCTGCTGAGCCCTTTCTCAGTGGG
ACTAGTTCGAACTATGTGGAGGAGATGTACTGTGCTTGGCTGGAAAACCCCAAAAGTGTACATAAGTCAT
GGGACATTTTTTTTCGCAACACGAATGCCGGAGCCCCACCGGGCACTGCCTACCAGAGTCCCCTTCCCCT
GAGCCGAGGCTCCCTGGCTGCTGTGGCCCATGCACAGTCCCTGGTAGAAGCACAGCCCAACGTGGACAAG
CTCGTGGAGGACCACCTGGCAGTGCAGTCGCTCATCAGGGCATATCAGATACGAGGGCACCATGTAGCAC
AGCTGGACCCCCTGGGGATTTTGGATGCTGATCTGGACTCCTCCGTGCCCGCTGACATTATCTCATCCAC
AGACAAACTTGGGTTCTATGGCCTGGATGAGTCTGACCTCGACAAGGTCTTCCACTTGCCCACCACCACT
TTCATCGGGGGACAGGAATCAGCACTTCCTCTGCGGGAGATCATCCGTCGGCTGGAGATGGCCTACTGCC
AGCATATTGGGGTGGAGTTCATGTTCATCAATGACCTGGAGCAGTGCCAGTGGATCCGGCAGAAGTTTGA
GACCCCTGGGATCATGCAGTTCACAAATGAGGAGAAACGGACCCTGCTGGCCAGGCTTGTGCGGTCCACC
AGGTTTGAGGAGTTCCTACAGCGGAAGTGGTCCTCTGAGAAGCGCTTTGGTCTAGAAGGCTGCGAGGTAC
TGATCCCTGCCCTCAAGACCATCATTGACAAGTCTAGTGAGAATGGCGTGGACTACGTGATCATGGGCAT
GCCACACAGAGGGCGGCTGAACGTGCTTGCAAATGTCATCAGGAAGGAGCTGGAACAGATCTTCTGTCAA
TTCGATTCAAAGCTGGAGGCAGCTGATGAGGGCTCCGGAGATGTGAAGTACCACCTGGGCATGTATCACC
GCAGGATCAATCGTGTCACCGACAGGAACATTACCTTGTCCTTGGTGGCCAACCCTTCCCACCTTGAGGC
CGCTGACCCCGTGGTGATGGGCAAGACCAAAGCCGAACAGTTTTACTGTGGCGACACTGAAGGGAAAAAG
GTAAGGCCCAGAGAGAGGCGTGCAAGGCAGATCGTCAAGGCCCCATGTTCCAGCATGGAGTTCCGCTCAC
CAACATAACCCAGAGCCCTGGGTGCATCTAGACTTTAAAAAAATATTTAAAGTCGGCCGGGCGCAGTGTC
TCACGCCTGTAATCCCAGCACTTTGGGAGGCCGAGGTGGGCAGATCACCTGAGTTCGGGAGTTGGAGACC
AGCCTGACCAACATGGAGAAACTCCATCTCTACTAAAAATACAAAATTAGCTGGGCGTGGTGGCGCGCGC
CTGTAATCCCAGCTACTCAGGAGGCTGAGGCAGGAGAATCGCTTGAACCCGGGAGGTGGAGGTTGCAGTG
AGCCGAGATTACGCCATTGCACTCCAGCCTGGGCCAACAAGAGCGAAACTCTGTCTCAAAGAAAAAAATA
AATAAATAAAAAA
NM_001165036.2 Homo sapiens oxoglutarate dehydrogenase (OGDH),
transcript variant 3, mRNA
(SEQ ID NO: 6)
ATTCGGGTGGAGCTGAGCCGGAGACAGGCAGTTGTGAAAAACTTCAGGACAAAAATGTTTCATTTAAGGA
CTTGTGCTGCTAAGTTGAGGCCATTGACGGCTTCCCAGACTGTTAAGACATTTTCACAAAACAGACCAGC
AGCAGCTAGGACATTTCAACAGATTCGGTGCTATTCTGCACCTGTTGCTGCTGAGCCCTTTCTCAGTGGG
ACTAGTTCGAACTATGTGGAGGAGATGTACTGTGCTTGGCTGGAAAACCCCAAAAGTGTACATAAGTCAT
GGGACATTTTTTTTCGCAACACGAATGCCGGAGCCCCACCGGGCACTGCCTACCAGAGTCCCCTTCCCCT
GAGCCGAGGCTCCCTGGCTGCTGTGGCCCATGCACAGTCCCTGGTAGAAGCACAGCCCAACGTGGACAAG
CTCGTGGAGGACCACCTGGCAGTGCAGTCGCTCATCAGGGCATATCAGGTCAGGGGTCACCACATTGCAA
AACTTGATCCTCTCGGAATTAGTTGTGTAAATTTTGATGATGCTCCAGTAACTGTTTCTTCAAACGTGGG
GTTCTATGGCCTGGATGAGTCTGACCTCGACAAGGTCTTCCACTTGCCCACCACCACTTTCATCGGGGGA
CAGGAATCAGCACTTCCTCTGCGGGAGATCATCCGTCGGCTGGAGATGGCCTACTGCCAGCATATTGGGG
TGGAGTTCATGTTCATCAATGACCTGGAGCAGTGCCAGTGGATCCGGCAGAAGTTTGAGACCCCTGGGAT
CATGCAGTTCACAAATGAGGAGAAACGGACCCTGCTGGCCAGGCTTGTGCGGTCCACCAGGTTTGAGGAG
TTCCTACAGCGGAAGTGGTCCTCTGAGAAGCGCTTTGGTCTAGAAGGCTGCGAGGTACTGATCCCTGCCC
TCAAGACCATCATTGACAAGTCTAGTGAGAATGGCGTGGACTACGTGATCATGGGCATGCCACACAGAGG
GCGGCTGAACGTGCTTGCAAATGTCATCAGGAAGGAGCTGGAACAGATCTTCTGTCAATTCGATTCAAAG
CTGGAGGCAGCTGATGAGGGCTCCGGAGATGTGAAGTACCACCTGGGCATGTATCACCGCAGGATCAATC
GTGTCACCGACAGGAACATTACCTTGTCCTTGGTGGCCAACCCTTCCCACCTTGAGGCCGCTGACCCCGT
GGTGATGGGCAAGACCAAAGCCGAACAGTTTTACTGTGGCGACACTGAAGGGAAAAAGGTCATGTCCATC
CTGTTGCATGGGGATGCTGCATTTGCTGGCCAGGGCATTGTGTACGAGACCTTCCACCTCAGCGACCTGC
CATCCTACACAACTCATGGCACCGTGCACGTGGTCGTCAACAACCAGATCGGCTTCACCACCGACCCTCG
GATGGCCCGCTCCTCCCCCTACCCCACTGACGTGGCCCGAGTGGTGAATGCCCCCATTTTCCACGTGAAC
TCAGATGACCCCGAGGCTGTCATGTACGTGTGCAAAGTGGCGGCCGAGTGGAGGAGCACCTTCCACAAGG
ACGTGGTTGTCGATTTGGTGTGTTACCGGCGCAACGGCCACAACGAGATGGATGAGCCCATGTTCACGCA
GCCGCTCATGTACAAGCAGATCCGCAAGCAGAAGCCTGTGTTACAGAAGTACGCTGAGCTGCTGGTGTCG
CAGGGTGTGGTCAACCAGCCTGAGTATGAGGAGGAAATTTCCAAGTATGATAAGATCTGTGAGGAAGCTT
TTGCCAGATCTAAAGATGAGAAGATCTTGCACATTAAGCACTGGCTGGACTCTCCCTGGCCTGGCTTCTT
CACCCTGGACGGGCAGCCCAGGAGCATGTCCTGCCCCTCCACGGGTCTGACGGAGGATATTCTGACACAC
ATCGGGAATGTGGCTAGTTCTGTGCCTGTGGAAAACTTTACTATTCATGGAGGGCTGAGCCGGATCTTGA
AGACTCGTGGGGAAATGGTGAAGAACCGGACTGTGGACTGGGCTCTAGCGGAGTACATGGCGTTTGGCTC
GCTCCTGAAGGAGGGCATCCACATTCGGCTGAGCGGCCAGGACGTGGAGCGGGGCACATTCAGCCACCGC
CACCATGTGCTCCATGACCAGAATGTGGACAAGAGAACCTGCATCCCCATGAACCATCTCTGGCCCAATC
AGGCCCCCTATACTGTGTGCAACAGCTCACTGTCTGAGTACGGCGTGCTGGGCTTTGAGCTGGGCTTCGC
CATGGCCAGTCCTAATGCCCTGGTCCTCTGGGAAGCCCAATTTGGTGACTTCCACAACACGGCCCAGTGT
ATCATCGACCAGTTCATCTGCCCGGGACAAGCCAAGTGGGTGCGGCAGAATGGCATCGTGTTGCTGCTGC
CCCATGGCATGGAGGGCATGGGTCCAGAACATTCCTCCGCCCGCCCAGAGCGGTTCTTGCAGATGTGCAA
CGATGACCCAGATGTCCTGCCAGACCTTAAAGAAGCCAACTTCGACATCAATCAGCTATATGACTGCAAT
TGGGTTGTTGTCAACTGCTCCACTCCTGGCAACTTCTTCCACGTGCTACGACGCCAGATCCTGCTGCCAT
TCCGGAAGCCGTTAATTATCTTCACCCCCAAATCCCTGTTGCGCCACCCCGAGGCCAGATCCAGCTTTGA
TGAGATGCTTCCAGGAACCCACTTCCAGCGGGTGATCCCAGAAGATGGCCCTGCAGCTCAGAACCCAGAA
AATGTCAAAAGGCTTCTCTTCTGCACCGGCAAAGTGTATTATGACCTCACCCGGGAGCGCAAAGCACGCG
ACATGGTGGGGCAGGTGGCCATCACAAGGATTGAGCAGCTGTCGCCATTCCCCTTTGACCTCCTGCTGAA
GGAGGTGCAGAAGTACCCCAATGCTGAGCTGGCCTGGTGCCAGGAGGAGCACAAGAACCAAGGCTACTAT
GACTACGTGAAGCCAAGACTTCGGACCACCATCAGCCGCGCCAAGCCCGTCTGGTATGCCGGCCGGGACC
CAGCGGCTGCTCCAGCCACCGGCAACAAGAAGACCCACCTGACGGAGCTGCAGCGCCTCCTGGACACGGC
CTTCGACCTGGACGTCTTCAAGAACTTCTCGTAGATGCTGCCTAGGGTTGCTTGGGCCACTGCCCTCTCC
ACACCCATGACTGCCCCTTGCTTCTCAACTAAAGAATAGTGCCTCAGCGCTGCCCACACCACCGCCCTCC
TCGCTGTGCCACCACCCCTCCCTCTGCTCTCATAGGAGTTAGGCTGTCGTCCCCCTCCAGTGCTTGGCTG
CCCCACAGGCCACACGCTGCCCAGGCTCTGCTGACTTCTGAGCAGTTTTCCAGGAGGCCGGGGGGAGCAG
GAGGAGGAAAGGTAGCCCCCGAGGGATGTCCTTGGGGAGGGGTCAGCTCTGGCCACAATCCTCCCCACCA
GTCTCACCCACTAGGATAGGAACTGGGCCTTGTGTGCTGGCTTCCGCTGTCACCCAGCAAGGCACAGGCT
CCTGTATTTGAGACTAGGATAGCTTCATCTTGAGCCTGAGCCTTAGAATCTGTAGAGGAGCCTGGAGTCG
GATCTAGCCATGGCTGGCAGAGGTTTCTAGGGTGGGCCCCAGCCGTGGCGTGAACTGAGGATGACCCGGG
GCAGCTGGCAGGAGAGAGCCTTGGCCTGACCTGGCACAGAAAGGGCAGCTTCAGTCTCTGCAGTGTCCAT
TATCTGCTGTTCCTTCGAGGGTTCCAGGCTGTGTGTGGGGCCCAAGCATGCCCCACCCACCCCTCCTGGG
CCCAGGCAGCACCTGGAGCCCACAGAGTCTGTGTGTAGCCAGGAAGCCCCGCTCAGGTAGCCACCACCGG
GGCACTGGCTGCTCTGTCTTGGTCCTGTTAACCCTCCACCTCCTCTCTTGGACTCCCTCCCCACCCCAAC
CACTCTTTCTTTCTCCTTTAACCCAATGGAGACTTTCTGATGCATCGTTTTCTTTGCTGTGCCAAAGCAG
GTCAGAAGAGGGAGAGGAGGGGCTGGGGGTGAGGGGCCAGGCCATGGCCAAGGGGCCAGCTGCCCCTCAT
TTATCACTCTGACCTTCACAGGGACAGATCTGATTTATTTATTTTGGTTAAAAAAAAAAAAAAGGAACAG
AAACAACTTTGCATTGCATTGGCTTGACCCATAAACTAAGTTATATCCGTG
NM_001363523.2 Homo sapiens oxoglutarate dehydrogenase (OGDH),
transcript variant 4, mRNA
(SEQ ID NO: 7)
ATTCGGGTGGAGCTGAGCCGGAGACAGGCAGTTGTGAAAAACTTCAGGACAAAAATGTTTCATTTAAGGA
CTTGTGCTGCTAAGTTGAGGCCATTGACGGCTTCCCAGACTGTTAAGACATTTTCACAAAACAGACCAGC
AGCAGCTAGGACATTTCAACAGATTCGGTGCTATTCTGCACCTGTTGCTGCTGAGCCCTTTCTCAGTGGG
ACTAGTTCGAACTATGTGGAGGAGATGTACTGTGCTTGGCTGGAAAACCCCAAAAGTGTACATAAGTCAT
GGGACATTTTTTTTCGCAACACGAATGCCGGAGCCCCACCGGGCACTGCCTACCAGAGTCCCCTTCCCCT
GAGCCGAGGCTCCCTGGCTGCTGTGGCCCATGCACAGTCCCTGGTAGAAGCACAGCCCAACGTGGACAAG
CTCGTGGAGGACCACCTGGCAGTGCAGTCGCTCATCAGGGCATATCAGGTCAGGGGTCACCACATTGCAA
AACTTGATCCTCTCGGAATTAGTTGTGTAAATTTTGATGATGCTCCAGTAACTGTTTCTTCAAACGTGGA
TCTTGCAGTTTTCAAGGAACGACTTCGAATGCTAACAGTAGGAGGGTTCTATGGCCTGGATGAGTCTGAC
CTCGACAAGGTCTTCCACTTGCCCACCACCACTTTCATCGGGGGACAGGAATCAGCACTTCCTCTGCGGG
AGATCATCCGTCGGCTGGAGATGGCCTACTGCCAGCATATTGGGGTGGAGTTCATGTTCATCAATGACCT
GGAGCAGTGCCAGTGGATCCGGCAGAAGTTTGAGACCCCTGGGATCATGCAGTTCACAAATGAGGAGAAA
CGGACCCTGCTGGCCAGGCTTGTGCGGTCCACCAGGTTTGAGGAGTTCCTACAGCGGAAGTGGTCCTCTG
AGAAGCGCTTTGGTCTAGAAGGCTGCGAGGTACTGATCCCTGCCCTCAAGACCATCATTGACAAGTCTAG
TGAGAATGGCGTGGACTACGTGATCATGGGCATGCCACACAGAGGGCGGCTGAACGTGCTTGCAAATGTC
ATCAGGAAGGAGCTGGAACAGATCTTCTGTCAATTCGATTCAAAGCTGGAGGCAGCTGATGAGGGCTCCG
GAGATGTGAAGTACCACCTGGGCATGTATCACCGCAGGATCAATCGTGTCACCGACAGGAACATTACCTT
GTCCTTGGTGGCCAACCCTTCCCACCTTGAGGCCGCTGACCCCGTGGTGATGGGCAAGACCAAAGCCGAA
CAGTTTTACTGTGGCGACACTGAAGGGAAAAAGGTCATGTCCATCCTGTTGCATGGGGATGCTGCATTTG
CTGGCCAGGGCATTGTGTACGAGACCTTCCACCTCAGCGACCTGCCATCCTACACAACTCATGGCACCGT
GCACGTGGTCGTCAACAACCAGATCGGCTTCACCACCGACCCTCGGATGGCCCGCTCCTCCCCCTACCCC
ACTGACGTGGCCCGAGTGGTGAATGCCCCCATTTTCCACGTGAACTCAGATGACCCCGAGGCTGTCATGT
ACGTGTGCAAAGTGGCGGCCGAGTGGAGGAGCACCTTCCACAAGGACGTGGTTGTCGATTTGGTGTGTTA
CCGGCGCAACGGCCACAACGAGATGGATGAGCCCATGTTCACGCAGCCGCTCATGTACAAGCAGATCCGC
AAGCAGAAGCCTGTGTTACAGAAGTACGCTGAGCTGCTGGTGTCGCAGGGTGTGGTCAACCAGCCTGAGT
ATGAGGAGGAAATTTCCAAGTATGATAAGATCTGTGAGGAAGCTTTTGCCAGATCTAAAGATGAGAAGAT
CTTGCACATTAAGCACTGGCTGGACTCTCCCTGGCCTGGCTTCTTCACCCTGGACGGGCAGCCCAGGAGC
ATGTCCTGCCCCTCCACGGGTCTGACGGAGGATATTCTGACACACATCGGGAATGTGGCTAGTTCTGTGC
CTGTGGAAAACTTTACTATTCATGGAGGGCTGAGCCGGATCTTGAAGACTCGTGGGGAAATGGTGAAGAA
CCGGACTGTGGACTGGGCTCTAGCGGAGTACATGGCGTTTGGCTCGCTCCTGAAGGAGGGCATCCACATT
CGGCTGAGCGGCCAGGACGTGGAGCGGGGCACATTCAGCCACCGCCACCATGTGCTCCATGACCAGAATG
TGGACAAGAGAACCTGCATCCCCATGAACCATCTCTGGCCCAATCAGGCCCCCTATACTGTGTGCAACAG
CTCACTGTCTGAGTACGGCGTGCTGGGCTTTGAGCTGGGCTTCGCCATGGCCAGTCCTAATGCCCTGGTC
CTCTGGGAAGCCCAATTTGGTGACTTCCACAACACGGCCCAGTGTATCATCGACCAGTTCATCTGCCCGG
GACAAGCCAAGTGGGTGCGGCAGAATGGCATCGTGTTGCTGCTGCCCCATGGCATGGAGGGCATGGGTCC
AGAACATTCCTCCGCCCGCCCAGAGCGGTTCTTGCAGATGTGCAACGATGACCCAGATGTCCTGCCAGAC
CTTAAAGAAGCCAACTTCGACATCAATCAGCTATATGACTGCAATTGGGTTGTTGTCAACTGCTCCACTC
CTGGCAACTTCTTCCACGTGCTACGACGCCAGATCCTGCTGCCATTCCGGAAGCCGTTAATTATCTTCAC
CCCCAAATCCCTGTTGCGCCACCCCGAGGCCAGATCCAGCTTTGATGAGATGCTTCCAGGAACCCACTTC
CAGCGGGTGATCCCAGAAGATGGCCCTGCAGCTCAGAACCCAGAAAATGTCAAAAGGCTTCTCTTCTGCA
CCGGCAAAGTGTATTATGACCTCACCCGGGAGCGCAAAGCACGCGACATGGTGGGGCAGGTGGCCATCAC
AAGGATTGAGCAGCTGTCGCCATTCCCCTTTGACCTCCTGCTGAAGGAGGTGCAGAAGTACCCCAATGCT
GAGCTGGCCTGGTGCCAGGAGGAGCACAAGAACCAAGGCTACTATGACTACGTGAAGCCAAGACTTCGGA
CCACCATCAGCCGCGCCAAGCCCGTCTGGTATGCCGGCCGGGACCCAGCGGCTGCTCCAGCCACCGGCAA
CAAGAAGACCCACCTGACGGAGCTGCAGCGCCTCCTGGACACGGCCTTCGACCTGGACGTCTTCAAGAAC
TTCTCGTAGATGCTGCCTAGGGTTGCTTGGGCCACTGCCCTCTCCACACCCATGACTGCCCCTTGCTTCT
CAACTAAAGAATAGTGCCTCAGCGCTGCCCACACCACCGCCCTCCTCGCTGTGCCACCACCCCTCCCTCT
GCTCTCATAGGAGTTAGGCTGTCGTCCCCCTCCAGTGCTTGGCTGCCCCACAGGCCACACGCTGCCCAGG
CTCTGCTGACTTCTGAGCAGTTTTCCAGGAGGCCGGGGGGAGCAGGAGGAGGAAAGGTAGCCCCCGAGGG
ATGTCCTTGGGGAGGGGTCAGCTCTGGCCACAATCCTCCCCACCAGTCTCACCCACTAGGATAGGAACTG
GGCCTTGTGTGCTGGCTTCCGCTGTCACCCAGCAAGGCACAGGCTCCTGTATTTGAGACTAGGATAGCTT
CATCTTGAGCCTGAGCCTTAGAATCTGTAGAGGAGCCTGGAGTCGGATCTAGCCATGGCTGGCAGAGGTT
TCTAGGGTGGGCCCCAGCCGTGGCGTGAACTGAGGATGACCCGGGGCAGCTGGCAGGAGAGAGCCTTGGC
CTGACCTGGCACAGAAAGGGCAGCTTCAGTCTCTGCAGTGTCCATTATCTGCTGTTCCTTCGAGGGTTCC
AGGCTGTGTGTGGGGCCCAAGCATGCCCCACCCACCCCTCCTGGGCCCAGGCAGCACCTGGAGCCCACAG
AGTCTGTGTGTAGCCAGGAAGCCCCGCTCAGGTAGCCACCACCGGGGCACTGGCTGCTCTGTCTTGGTCC
TGTTAACCCTCCACCTCCTCTCTTGGACTCCCTCCCCACCCCAACCACTCTTTCTTTCTCCTTTAACCCA
ATGGAGACTTTCTGATGCATCGTTTTCTTTGCTGTGCCAAAGCAGGTCAGAAGAGGGAGAGGAGGGGCTG
GGGGTGAGGGGCCAGGCCATGGCCAAGGGGCCAGCTGCCCCTCATTTATCACTCTGACCTTCACAGGGAC
AGATCTGATTTATTTATTTTGGTTAAAAAAAAAAAAAAGGAACAGAAACAACTTTGCATTGCATTGGCTT
GACCCATAAACTAAGTTATATCCGTG

Exemplary mRNA sequences of DLST are provided below, represented by SEQ ID NOs: 8-9:

NM_001933.5 Homo sapiens dihydrolipoamide S-succinyltransferase (DLST),
transcript variant 1, mRNA; nuclear gene for mitochondrial product
(SEQ ID NO: 8)
ATATCCGGTGTCCGCCCGCCCTCGGCTCCTCCGCCGTGATGCTGTCCCGATCCCGCTGTGTGTCTCGGGC
GTTCAGCCGCTCGCTCTCCGCCTTCCAGAAGGGGAACTGCCCTCTAGGGAGACGTTCCCTGCCTGGGGTC
TCCTTATGCCAGGGACCAGGTTACCCTAACAGCAGGAAGGTTGTCATTAACAACAGTGTCTTCAGTGTTC
GCTTTTTCAGAACTACAGCTGTATGCAAGGATGACTTGGTTACAGTCAAAACCCCAGCGTTTGCAGAATC
TGTCACAGAGGGAGATGTCAGGTGGGAGAAAGCTGTTGGAGACACAGTTGCAGAAGATGAAGTGGTTTGT
GAGATTGAAACTGACAAGACATCTGTGCAGGTTCCATCACCAGCAAATGGCGTGATTGAAGCTCTTTTGG
TACCTGATGGGGGAAAAGTCGAAGGAGGCACTCCACTTTTCACACTCAGGAAAACTGGTGCTGCTCCTGC
TAAGGCCAAGCCGGCTGAAGCTCCTGCTGCTGCAGCCCCAAAAGCAGAACCTACAGCAGCGGCAGTTCCT
CCCCCTGCAGCACCCATACCCACTCAGATGCCACCGGTGCCCTCGCCCTCACAGCCTCCTTCTGGCAAAC
CTGTGTCTGCAGTAAAACCCACTGTTGCCCCACCACTAGCTGAGCCAGGAGCTGGCAAAGGTCTGCGTTC
AGAACATCGGGAGAAAATGAACAGGATGCGGCAGCGCATTGCTCAGCGTCTGAAGGAGGCCCAGAATACA
TGTGCAATGCTGACAACTTTTAATGAGATTGACATGAGTAACATCCAGGAGATGAGGGCTCGGCACAAAG
AGGCTTTTTTGAAGAAACATAACCTCAAACTAGGCTTCATGTCGGCATTTGTGAAGGCCTCAGCCTTTGC
CTTGCAGGAACAGCCTGTTGTAAATGCAGTGATTGACGACACAACCAAAGAGGTGGTGTATAGGGATTAT
ATTGACATCAGTGTTGCAGTGGCCACCCCACGGGGTCTGGTGGTTCCAGTCATCAGGAATGTGGAAGCTA
TGAATTTTGCAGATATTGAACGGACCATCACTGAACTGGGAGAGAAGGCCCGAAAGAATGAACTTGCCAT
TGAAGATATGGATGGCGGTACCTTCACCATTAGCAATGGAGGCGTTTTTGGCTCGCTCTTTGGAACACCC
ATTATCAACCCCCCTCAGTCTGCCATCCTGGGGATGCATGGCATCTTTGACAGGCCAGTGGCTATAGGAG
GCAAGGTAGAGGTGCGGCCCATGATGTACGTGGCACTGACCTATGATCACCGGCTGATTGATGGCAGAGA
GGCTGTGACTTTCCTCCGCAAAATCAAGGCAGCGGTAGAGGATCCCAGAGTCCTCCTCCTGGATCTTTAG
GAGGAACCCACACACCCTACAAGTTGATCATGCAGGAACTGAAAACCAGTCTTCTCCCTGTCCCCTCATG
GGTCCCGGGTTAGCCTGGTGACAGGCAGACACATGCTGTTGGCCTCAAGCAAGGAAGCAGAGCACTGTGT
AACCAGCAGTCACAGGTCTTTTCTTGGCGTTCCTGCCAGGCTCTCCCTCTCTGCACCTGTCTCATAGCCT
CGAATATCTTAATTCCTTAGGCTTAAGAGAGAGAGCCTTAATGGATGCTCATTCATATTCCTGCCTTTCT
TCCATCAGCTCTCTGCAAAGATGATTTTGCTTTTCCCTAGTGCTGGTATACTATAGAGAAACCCCTGGGG
ACCATGTGATTAAGTTCCTATCTTTTGAAAGTTTGTTCTGCAGAGACTTCTAGGAGGATGCTGTGCCTCC
CAAGCTCAGAGCAGCCTCTGTCCTGGCTGTGCACATTCTCCCTTGATTCCACTTGTGTGGAGGGATTGAA
CACAGGCAAAGAGGTGCTGCTTTGCTTCTTCAATGGCACCTTCATTCTCCGTTGTCATTGACTTCAAGAT
GCCTCTTCTACCTCTTCCAGGAAGCACAGGCCAGGGGATCTGGGTGTGTGAGTGGGAGGAGAGGGCAGAG
GTCCCCTGAGGTCATGCATTGTAATCATCATAGAAGGAGAGCCCAGGCCTGCCCTCACGCTCTCCATCAT
AGGCTGACACCAAGAAGACTCGTCTTGGCACAATCTCACACAGCTGGGGCTGTAGCAACCCTTTCCAACC
CCTTTGCTGGTTGCTGGGCCTCATTCTAGCACCTTGTTCTTAGAGCAGATTCTAGCACATCATGGCAGTG
GGACCAAGCGTGGTCCCGAGGAAGGGCCAGAGCCTGGTAGAGACTAGGGAAGGGAGGTCTCCTCTAGACT
GACTCACATTGCCTTGAGCTTTTCAGTTAAGTTGCTGTAAGCACCTGGGCTGAGGAGGCAGTTTTTGTTC
CTTCCTGCGTTATAGCGGGGCCTTGTCTCTTCCTCTGCAGGACACAGATCTGGAGGACGTGGACTGGGGT
AGGAAACCACCCTGAGGGTGTTAGTACCTAGTGGTGAAACGGATGAGGTCATTTCTAAGGTGTGTTGCCC
GTGGATCTGGGCACAATCATTGGAATTCCTTGGAGCCACTGGGATTCATGGCTTTGTATCCAACTGCATC
CAGGCCTGAGGCTGCTGACGTTTGACACCAGGGCCAGTAGAGAGTGCCCTTTTGTATCTTAAGCCAAGAA
GTGAGGCCTGGGGGTGGGGGAGGGGGGAAGGGGTGGGAGCCAATACTGAGTGCCTGCAGCATCTACTACT
CTGTCTTCACTATTCAGAACTTGTAACTAAAGTATTTAAAGAAACTGATTTTAAATGCAAATTAAAGGGC
AGATATTCTCAAA
NM_001244883.2 Homo sapiens dihydrolipoamide S-succinyltransferase
(DLST), transcript variant 2, mRNA; nuclear gene for mitochondrial
product
(SEQ ID NO: 9)
ATATCCGGTGTCCGCCCGCCCTCGGCTCCTCCGCCGTGATGCTGTCCCGATCCCGCTGTGTGTCTCGGGC
GTTCAGCCGCTCGCTCTCCGCCTTCCAGAAGGGGAACTGCCCTCTAGGGAGACGTTCCCTGCCTGGGGTC
TCCTTATGCCAGGGACCAGGTTACCCTAACAGCAGGAAGGTTGTCATTAACAACAGTGTCTTCAGTGTTC
GCTTTTTCAGAACTACAGCTGTATGCAAGGATGACTTGGTTACAGTCAAAACCCCAGCGTTTGCAGAATC
TGTCACAGAGGGAGATGTCAGGTGGGAGAAAGCTGTTGGAGACACAGTTGCAGAAGATGAAGTGGTTTGT
GAGATTGAAACTGACAAGACATCTGTGCAGGTTCCATCACCAGCAAATGGCGTGATTGAAGCTCTTTTGG
TACCTGATGGGGGAAAAGTCGAAGGAGGCACTCCACTTTTCACACTCAGGAAAACTGGTGGTAAAGAAGT
TCTCCTGGTGGTCAAGGTCTCCAGTGTTCCCTCTTGGGATTGGGACTGAGCATAATGTGCTAATTCCCCG
ATATGTCAAAGACTCTTGTCATTCCAGCTGCCCTTAGTTGAGGGAATAAGGGGTAAATTTATATATAGTG
TGAACTTCAAATGTCAAATTCTAAAAGAAAAGTGTATTTTTTAAAAAATAAACCTTATACTCTGTTCTTT
CCATGGGTGTTTCTTAGCAGGAGCTGAGAAACTGGACCTTTTCTTATAGATATACAGATTGAGCATACCT
AATCTGAAAATTCAAAATCTGAAATGCTCCAAAACTGAAAATCCAAAATCTGAAATGCTTCAAAACTTTT
TGAGCACTGACAGAATGCTCAAAGGAAATGCTCATTGTCTTTTATGGGGAATTCAGTGTTGTTTCTTTTC
ACATCTGTTCTCTCATATCTCTAGGAAGGGTTGGTATTTCTTTTGGGAAGAAATGAGAAACGAATTTGTA
CTGGACCTGATAGGATTAGAGATTAATAACTTTATCTGACTCTTAGTTAAATTTTATAACCACTAAAAAG
TCTATTTCTTTTTCCTAAGCGAGAATCGTACCCGTAGACCAACGAGTCACAGAAGTCTATTTCTTTTTCA
CTGTTACACTAATAAGGCAGATAGACCGCATGATTGAGGTTAGTCAGGCAAGACCAGAAGCTCAGAGTAG
AAAACATGTATGGTAGAAGGCTGGGAAATAAGGTAGTCCTCCTGTTGCAGAAGTTTGTCTTAGCACTAAC
CCTGGATATAATGTCCAGATGGGTGAAAAACAGAAACAAACATTTCATTTAAAGTTGTGAGATTATTACT
TGGCCACTTCAGTGATTTATGAGTCATCCTGGATGAAGTGC

Exemplary mRNA sequences of DLD are provided below, represented by SEQ ID NOs: 10-13:

NM_000108.5 Homo sapiens dihydrolipoamide dehydrogenase (DLD),
transcript variant 1, mRNA; nuclear gene for mitochondrial product
(SEQ ID NO: 10)
CTTGGCGGAGCGGCGGAGGCGCCCAGCGGAGGTGAAAGTATTGGCGGAAAGGAAAATACAGCGGAAAAAT
GCAGAGCTGGAGTCGTGTGTACTGCTCCTTGGCCAAGAGAGGCCATTTCAATCGAATATCTCATGGCCTA
CAGGGACTTTCTGCAGTGCCTCTGAGAACTTACGCAGATCAGCCGATTGATGCTGATGTAACAGTTATAG
GTTCTGGTCCTGGAGGATATGTTGCTGCTATTAAAGCTGCCCAGTTAGGCTTCAAGACAGTCTGCATTGA
GAAAAATGAAACACTTGGTGGAACATGCTTGAATGTTGGTTGTATTCCTTCTAAGGCTTTATTGAACAAC
TCTCATTATTACCATATGGCCCATGGAAAAGATTTTGCATCTAGAGGAATTGAAATGTCCGAAGTTCGCT
TGAATTTAGACAAGATGATGGAGCAGAAGAGTACTGCAGTAAAAGCTTTAACAGGTGGAATTGCCCACTT
ATTCAAACAGAATAAGGTTGTTCATGTCAATGGATATGGAAAGATAACTGGCAAAAATCAAGTCACTGCT
ACGAAAGCTGATGGCGGCACTCAGGTTATTGATACAAAGAACATTCTTATAGCCACGGGTTCAGAAGTTA
CTCCTTTTCCTGGAATCACGATAGATGAAGATACAATAGTGTCATCTACAGGTGCTTTATCTTTAAAAAA
AGTTCCAGAAAAGATGGTTGTTATTGGTGCAGGAGTAATAGGTGTAGAATTGGGTTCAGTTTGGCAAAGA
CTTGGTGCAGATGTGACAGCAGTTGAATTTTTAGGTCATGTAGGTGGAGTTGGAATTGATATGGAGATAT
CTAAAAACTTTCAACGCATCCTTCAAAAACAGGGGTTTAAATTTAAATTGAATACAAAGGTTACTGGTGC
TACCAAGAAGTCAGATGGAAAAATTGATGTTTCTATTGAAGCTGCTTCTGGTGGTAAAGCTGAAGTTATC
ACTTGTGATGTACTCTTGGTTTGCATTGGCCGACGACCCTTTACTAAGAATTTGGGACTAGAAGAGCTGG
GAATTGAACTAGATCCCAGAGGTAGAATTCCAGTCAATACCAGATTTCAAACTAAAATTCCAAATATCTA
TGCCATTGGTGATGTAGTTGCTGGTCCAATGCTGGCTCACAAAGCAGAGGATGAAGGCATTATCTGTGTT
GAAGGAATGGCTGGTGGTGCTGTGCACATTGACTACAATTGTGTGCCATCAGTGATTTACACACACCCTG
AAGTTGCTTGGGTTGGCAAATCAGAAGAGCAGTTGAAAGAAGAGGGTATTGAGTACAAAGTTGGGAAATT
CCCATTTGCTGCTAACAGCAGAGCTAAGACAAATGCTGACACAGATGGCATGGTGAAGATCCTTGGGCAG
AAATCGACAGACAGAGTACTGGGAGCACATATTCTTGGACCAGGTGCTGGAGAAATGGTAAATGAAGCTG
CTCTTGCTTTGGAATATGGAGCATCCTGTGAAGATATAGCTAGAGTCTGTCATGCACATCCGACCTTATC
AGAAGCTTTTAGAGAAGCAAATCTTGCTGCGTCATTTGGCAAATCAATCAACTTTTGAATTAGAAGATTA
TATATATTTTTTTCTGAAATTTCCTGGGAGCTTTTGTAGAAGTCACATTCCTGAACAGGATATTCTCACA
GCTCCAAGAATTTCTAGGACTGAATTATGAAACTTTTGGAAGGTATTTAATAGGTTTGGACAAAATGGAA
TACTCTTATATCTATATTTTACATAAATTTAGTATTTTGTTTCAGTGCACTAATGTGTAAGACAAAAAGC
TACTTATTGTAGCATCCTGGAATATCTCCGTCAACTCATATTTTCATGCTGTTCATGAAAGATTCAATGC
CCCTGAATTTAAATAGCTTTTTTCTCTGATACAGAAAAGTTGAATTTTACATGGCTGGAGCTAGAATTTG
ATATGTGAACAGTTGTGTTTGAAGCACAGTGATCAAGTTATTTTTAATTTGGTTTTCACATTGGAAACAA
GTCAGTCATTCAGATATGATTCAAATGTCTATAAACCGAACTGATGTAAGTAAACGGTCTCTCACTTGTT
TTATTTAACCTCTAAATTCTTTCATTTTAGGGGTAGCATTTGTGTTGAAGAGGTTTTAAAGCTTCCATTG
TTGTCTGCAACTCTGAAGGGTAATTATATAGTTACCCAAATTAAGAGAGTCTATTTACGGAACTCAAATA
CGTGGGCATTCAAATGTATTACAGTGGGGAATGAAGATACTGAAATAAACGTCTTAAATATTCATTTACT
GGTTATCATGAGTACGTGTTGAGATGGTCATAGTTTTTTTTATGACTACTTCTAGTGTATATTCTAATTT
CTTTTCTAGGCCTGAATGTATCTTTATTTTCATGTTATAGGACAATATTAAGGCATTTTAAAGGTCATCA
TCCTTTCATCTATTTTAGATACACCTACTAAATGTTTAATATATACTTTTGGAGAAGTACAACATAAGGG
AGTCTTTAATCTGTGTTTTCCTTGGCTGGGTTAATGACTGTTTATTTAAAGAGTGTTGTAAAATTGGATG
TGTGGTGTTTAAAATGGCCATGTCCTGAGGAAACTTAAGTAACAAAGTACTAAATGCTAAGTAGGCTTTT
GCATATTGTAACTAAATTTAAGAATAATTCAGATTAAGTAGTTCTGAAATTTGGTATAGATAGCATAGAT
TGTCTCATGCTCATGAGTGACATAATGACCCTGGATTCTGTTACATACTTCTAAAGAAAATTGATTGTTG
TCTTAGGAGGCAGTTAACTTGGCTGAACACCAACTCCACACTCTGTCTTGTTTGTAGGTGGCAGCAGCTG
AAATCTCTTCTCAGTTGTTTTAGCTTTAGCTATGCTGCTGGAAGTCTTTCCCATGCAAGTGTGTAGTTCA
GGGGTCAACCAGAGTTTGGGCAGAAGGAAGTCTGCCCCTTCTGTGCCTCCTGTTTTTTGGGGGTTTCCCC
TTTATGTTCCAGCTGTTGTGGTTGCCCCATATTCTGCCTTCTGATCCTTAACCAATAAAACTTGGCTTTT
GTTTCCCCCTCAAGTGAGAACCCGTTAAAAATGAGACATTGAGCCAGTGCTGTTCACTTTTTAAGTGCCA
ACTTCCCTCTACTTTCCACTTGTTTATAGTTGTTTCCAGTGCCTTTAGTTTTTTCTAAAATATATTTGTT
CAGAGTTTGCAGTTGCTATCAGCAGGAGGGTTGGTCTGATATCTGTGTGCTACTTTGCCATTATTGGAAG
TGAACTCTGCATCTTTTTAAAAATTTGAAATCCCGGTATCATGTGAAGTGCTGTTTATGTAAATCTCAAC
ATATCCCTTACTCAGGGAAAAAAAAGTTTTTAGTTAGGGAATAGTGAAATATAATTTAATATGGAATTCT
AGCTGTAGAGTTAAATCCATCTTTAAGTGTTTACATTCAGTATGAGAATGCAAATTTATCTGTATGGGGA
ATAAAGTCCTAGGAATAAAACAAGTTTTAAGTGTTCA
NM_001289750.1 Homo sapiens dihydrolipoamide dehydrogenase (DLD),
transcript variant 2, mRNA; nuclear gene for mitochondrial product
(SEQ ID NO: 11)
AGAACCGCGCGGGCCAATCGCGCTGCTCCCGGGTGATGACGTAGGCTGCGCCTGTGCATGCGCAGGGAGG
GGAGACCTTGGCGGAGCGGCGGAGGCGCCCAGCGGAGGTGAAAGTATTGGCGGAAAGGAAAATACAGCGG
AAAAATGCAGAGCTGGAGTCGTGTGTACTGCTCCTTGGCCAAGAGAGGCCATTTCAATCGAATATCTCAT
GGCCTACAGGGACTTTCTGCAGTGCCTCTGAGAACTTACGCAGATCAGCCGAGCTTTATTGAACAACTCT
CATTATTACCATATGGCCCATGGAAAAGATTTTGCATCTAGAGGAATTGAAATGTCCGAAGTTCGCTTGA
ATTTAGACAAGATGATGGAGCAGAAGAGTACTGCAGTAAAAGCTTTAACAGGTGGAATTGCCCACTTATT
CAAACAGAATAAGGTTGTTCATGTCAATGGATATGGAAAGATAACTGGCAAAAATCAAGTCACTGCTACG
AAAGCTGATGGCGGCACTCAGGTTATTGATACAAAGAACATTCTTATAGCCACGGGTTCAGAAGTTACTC
CTTTTCCTGGAATCACGATAGATGAAGATACAATAGTGTCATCTACAGGTGCTTTATCTTTAAAAAAAGT
TCCAGAAAAGATGGTTGTTATTGGTGCAGGAGTAATAGGTGTAGAATTGGGTTCAGTTTGGCAAAGACTT
GGTGCAGATGTGACAGCAGTTGAATTTTTAGGTCATGTAGGTGGAGTTGGAATTGATATGGAGATATCTA
AAAACTTTCAACGCATCCTTCAAAAACAGGGGTTTAAATTTAAATTGAATACAAAGGTTACTGGTGCTAC
CAAGAAGTCAGATGGAAAAATTGATGTTTCTATTGAAGCTGCTTCTGGTGGTAAAGCTGAAGTTATCACT
TGTGATGTACTCTTGGTTTGCATTGGCCGACGACCCTTTACTAAGAATTTGGGACTAGAAGAGCTGGGAA
TTGAACTAGATCCCAGAGGTAGAATTCCAGTCAATACCAGATTTCAAACTAAAATTCCAAATATCTATGC
CATTGGTGATGTAGTTGCTGGTCCAATGCTGGCTCACAAAGCAGAGGATGAAGGCATTATCTGTGTTGAA
GGAATGGCTGGTGGTGCTGTGCACATTGACTACAATTGTGTGCCATCAGTGATTTACACACACCCTGAAG
TTGCTTGGGTTGGCAAATCAGAAGAGCAGTTGAAAGAAGAGGGTATTGAGTACAAAGTTGGGAAATTCCC
ATTTGCTGCTAACAGCAGAGCTAAGACAAATGCTGACACAGATGGCATGGTGAAGATCCTTGGGCAGAAA
TCGACAGACAGAGTACTGGGAGCACATATTCTTGGACCAGGTGCTGGAGAAATGGTAAATGAAGCTGCTC
TTGCTTTGGAATATGGAGCATCCTGTGAAGATATAGCTAGAGTCTGTCATGCACATCCGACCTTATCAGA
AGCTTTTAGAGAAGCAAATCTTGCTGCGTCATTTGGCAAATCAATCAACTTTTGAATTAGAAGATTATAT
ATATTTTTTTCTGAAATTTCCTGGGAGCTTTTGTAGAAGTCACATTCCTGAACAGGATATTCTCACAGCT
CCAAGAATTTCTAGGACTGAATTATGAAACTTTTGGAAGGTATTTAATAGGTTTGGACAAAATGGAATAC
TCTTATATCTATATTTTACATAAATTTAGTATTTTGTTTCAGTGCACTAATGTGTAAGACAAAAAGCTAC
TTATTGTAGCATCCTGGAATATCTCCGTCAACTCATATTTTCATGCTGTTCATGAAAGATTCAATGCCCC
TGAATTTAAATAGCTTTTTTCTCTGATACAGAAAAGTTGAATTTTACATGGCTGGAGCTAGAATTTGATA
TGTGAACAGTTGTGTTTGAAGCACAGTGATCAAGTTATTTTTAATTTGGTTTTCACATTGGAAACAAGTC
AGTCATTCAGATATGATTCAAATGTCTATAAACCGAACTGATGTAAGTAAACGGTCTCTCACTTGTTTTA
TTTAACCTCTAAATTCTTTCATTTTAGGGGTAGCATTTGTGTTGAAGAGGTTTTAAAGCTTCCATTGTTG
TCTGCAACTCTGAAGGGTAATTATATAGTTACCCAAATTAAGAGAGTCTATTTACGGAACTCAAATACGT
GGGCATTCAAATGTATTACAGTGGGGAATGAAGATACTGAAATAAACGTCTTAAATATTCATTTACTGGT
TATCATGAGTACGTGTTGAGATGGTCATAGTTTTTTTTATGACTACTTCTAGTGTATATTCTAATTTCTT
TTCTAGGCCTGAATGTATCTTTATTTTCATGTTATAGGACAATATTAAGGCATTTTAAAGGTCATCATCC
TTTCATCTATTTTAGATACACCTACTAAATGTTTAATATATACTTTTGGAGAAGTACAACATAAGGGAGT
CTTTAATCTGTGTTTTCCTTGGCTGGGTTAATGACTGTTTATTTAAAGAGTGTTGTAAAATTGGATGTGT
GGTGTTTAAAATGGCCATGTCCTGAGGAAACTTAAGTAACAAAGTACTAAATGCTAAGTAGGCTTTTGCA
TATTGTAACTAAATTTAAGAATAATTCAGATTAAGTAGTTCTGAAATTTGGTATAGATAGCATAGATTGT
CTCATGCTCATGAGTGACATAATGACCCTGGATTCTGTTACATACTTCTAAAGAAAATTGATTGTTGTCT
TAGGAGGCAGTTAACTTGGCTGAACACCAACTCCACACTCTGTCTTGTTTGTAGGTGGCAGCAGCTGAAA
TCTCTTCTCAGTTGTTTTAGCTTTAGCTATGCTGCTGGAAGTCTTTCCCATGCAAGTGTGTAGTTCAGGG
GTCAACCAGAGTTTGGGCAGAAGGAAGTCTGCCCCTTCTGTGCCTCCTGTTTTTTGGGGGTTTCCCCTTT
ATGTTCCAGCTGTTGTGGTTGCCCCATATTCTGCCTTCTGATCCTTAACCAATAAAACTTGGCTTTTGTT
TCCCCCTCAAGTGAGAACCCGTTAAAAATGAGACATTGAGCCAGTGCTGTTCACTTTTTAAGTGCCAACT
TCCCTCTACTTTCCACTTGTTTATAGTTGTTTCCAGTGCCTTTAGTTTTTTCTAAAATATATTTGTTCAG
AGTTTGCAGTTGCTATCAGCAGGAGGGTTGGTCTGATATCTGTGTGCTACTTTGCCATTATTGGAAGTGA
ACTCTGCATCTTTTTAAAAATTTGAAATCCCGGTATCATGTGAAGTGCTGTTTATGTAAATCTCAACATA
TCCCTTACTCAGGGAAAAAAAAGTTTTTAGTTAGGGAATAGTGAAATATAATTTAATATGGAATTCTAGC
TGTAGAGTTAAATCCATCTTTAAGTGTTTACATTCAGTATGAGAATGCAAATTTATCTGTATGGGGAATA
AAGTCCTAGGAATAAAACAAGTTTTAAGTGTTCA
NM_001289751.1 Homo sapiens dihydrolipoamide dehydrogenase (DLD),
transcript variant 3, mRNA; nuclear gene for mitochondrial product
(SEQ ID NO: 12)
AGAACCGCGCGGGCCAATCGCGCTGCTCCCGGGTGATGACGTAGGCTGCGCCTGTGCATGCGCAGGGAGG
GGAGACCTTGGCGGAGCGGCGGAGGCGCCCAGCGGAGGTGAAAGTATTGGCGGAAAGGAAAATACAGCGG
AAAAATGCAGAGCTGGAGTCGTGTGTACTGCTCCTTGGCCAAGAGAGGCCATTTCAATCGAATATCTCAT
GGCCTACAGGGACTTTCTGCAGTGCCTCTGAGAACTTACGCAGATCAGCCGATTGATGCTGATGTAACAG
TTATAGGTTCTGGTCCTGGAGGATATGTTGCTGCTATTAAAGCTGCCCAGTTAGGCTTCAAGGCTTTATT
GAACAACTCTCATTATTACCATATGGCCCATGGAAAAGATTTTGCATCTAGAGGAATTGAAATGTCCGAA
GTTCGCTTGAATTTAGACAAGATGATGGAGCAGAAGAGTACTGCAGTAAAAGCTTTAACAGGTGGAATTG
CCCACTTATTCAAACAGAATAAGGTTGTTCATGTCAATGGATATGGAAAGATAACTGGCAAAAATCAAGT
CACTGCTACGAAAGCTGATGGCGGCACTCAGGTTATTGATACAAAGAACATTCTTATAGCCACGGGTTCA
GAAGTTACTCCTTTTCCTGGAATCACGATAGATGAAGATACAATAGTGTCATCTACAGGTGCTTTATCTT
TAAAAAAAGTTCCAGAAAAGATGGTTGTTATTGGTGCAGGAGTAATAGGTGTAGAATTGGGTTCAGTTTG
GCAAAGACTTGGTGCAGATGTGACAGCAGTTGAATTTTTAGGTCATGTAGGTGGAGTTGGAATTGATATG
GAGATATCTAAAAACTTTCAACGCATCCTTCAAAAACAGGGGTTTAAATTTAAATTGAATACAAAGGTTA
CTGGTGCTACCAAGAAGTCAGATGGAAAAATTGATGTTTCTATTGAAGCTGCTTCTGGTGGTAAAGCTGA
AGTTATCACTTGTGATGTACTCTTGGTTTGCATTGGCCGACGACCCTTTACTAAGAATTTGGGACTAGAA
GAGCTGGGAATTGAACTAGATCCCAGAGGTAGAATTCCAGTCAATACCAGATTTCAAACTAAAATTCCAA
ATATCTATGCCATTGGTGATGTAGTTGCTGGTCCAATGCTGGCTCACAAAGCAGAGGATGAAGGCATTAT
CTGTGTTGAAGGAATGGCTGGTGGTGCTGTGCACATTGACTACAATTGTGTGCCATCAGTGATTTACACA
CACCCTGAAGTTGCTTGGGTTGGCAAATCAGAAGAGCAGTTGAAAGAAGAGGGTATTGAGTACAAAGTTG
GGAAATTCCCATTTGCTGCTAACAGCAGAGCTAAGACAAATGCTGACACAGATGGCATGGTGAAGATCCT
TGGGCAGAAATCGACAGACAGAGTACTGGGAGCACATATTCTTGGACCAGGTGCTGGAGAAATGGTAAAT
GAAGCTGCTCTTGCTTTGGAATATGGAGCATCCTGTGAAGATATAGCTAGAGTCTGTCATGCACATCCGA
CCTTATCAGAAGCTTTTAGAGAAGCAAATCTTGCTGCGTCATTTGGCAAATCAATCAACTTTTGAATTAG
AAGATTATATATATTTTTTTCTGAAATTTCCTGGGAGCTTTTGTAGAAGTCACATTCCTGAACAGGATAT
TCTCACAGCTCCAAGAATTTCTAGGACTGAATTATGAAACTTTTGGAAGGTATTTAATAGGTTTGGACAA
AATGGAATACTCTTATATCTATATTTTACATAAATTTAGTATTTTGTTTCAGTGCACTAATGTGTAAGAC
AAAAAGCTACTTATTGTAGCATCCTGGAATATCTCCGTCAACTCATATTTTCATGCTGTTCATGAAAGAT
TCAATGCCCCTGAATTTAAATAGCTTTTTTCTCTGATACAGAAAAGTTGAATTTTACATGGCTGGAGCTA
GAATTTGATATGTGAACAGTTGTGTTTGAAGCACAGTGATCAAGTTATTTTTAATTTGGTTTTCACATTG
GAAACAAGTCAGTCATTCAGATATGATTCAAATGTCTATAAACCGAACTGATGTAAGTAAACGGTCTCTC
ACTTGTTTTATTTAACCTCTAAATTCTTTCATTTTAGGGGTAGCATTTGTGTTGAAGAGGTTTTAAAGCT
TCCATTGTTGTCTGCAACTCTGAAGGGTAATTATATAGTTACCCAAATTAAGAGAGTCTATTTACGGAAC
TCAAATACGTGGGCATTCAAATGTATTACAGTGGGGAATGAAGATACTGAAATAAACGTCTTAAATATTC
ATTTACTGGTTATCATGAGTACGTGTTGAGATGGTCATAGTTTTTTTTATGACTACTTCTAGTGTATATT
CTAATTTCTTTTCTAGGCCTGAATGTATCTTTATTTTCATGTTATAGGACAATATTAAGGCATTTTAAAG
GTCATCATCCTTTCATCTATTTTAGATACACCTACTAAATGTTTAATATATACTTTTGGAGAAGTACAAC
ATAAGGGAGTCTTTAATCTGTGTTTTCCTTGGCTGGGTTAATGACTGTTTATTTAAAGAGTGTTGTAAAA
TTGGATGTGTGGTGTTTAAAATGGCCATGTCCTGAGGAAACTTAAGTAACAAAGTACTAAATGCTAAGTA
GGCTTTTGCATATTGTAACTAAATTTAAGAATAATTCAGATTAAGTAGTTCTGAAATTTGGTATAGATAG
CATAGATTGTCTCATGCTCATGAGTGACATAATGACCCTGGATTCTGTTACATACTTCTAAAGAAAATTG
ATTGTTGTCTTAGGAGGCAGTTAACTTGGCTGAACACCAACTCCACACTCTGTCTTGTTTGTAGGTGGCA
GCAGCTGAAATCTCTTCTCAGTTGTTTTAGCTTTAGCTATGCTGCTGGAAGTCTTTCCCATGCAAGTGTG
TAGTTCAGGGGTCAACCAGAGTTTGGGCAGAAGGAAGTCTGCCCCTTCTGTGCCTCCTGTTTTTTGGGGG
TTTCCCCTTTATGTTCCAGCTGTTGTGGTTGCCCCATATTCTGCCTTCTGATCCTTAACCAATAAAACTT
GGCTTTTGTTTCCCCCTCAAGTGAGAACCCGTTAAAAATGAGACATTGAGCCAGTGCTGTTCACTTTTTA
AGTGCCAACTTCCCTCTACTTTCCACTTGTTTATAGTTGTTTCCAGTGCCTTTAGTTTTTTCTAAAATAT
ATTTGTTCAGAGTTTGCAGTTGCTATCAGCAGGAGGGTTGGTCTGATATCTGTGTGCTACTTTGCCATTA
TTGGAAGTGAACTCTGCATCTTTTTAAAAATTTGAAATCCCGGTATCATGTGAAGTGCTGTTTATGTAAA
TCTCAACATATCCCTTACTCAGGGAAAAAAAAGTTTTTAGTTAGGGAATAGTGAAATATAATTTAATATG
GAATTCTAGCTGTAGAGTTAAATCCATCTTTAAGTGTTTACATTCAGTATGAGAATGCAAATTTATCTGT
ATGGGGAATAAAGTCCTAGGAATAAAACAAGTTTTAAGTGTTCA
NM_001289752.1 Homo sapiens dihydrolipoamide dehydrogenase (DLD),
transcript variant 4, mRNA; nuclear gene for mitochondrial product
(SEQ ID NO: 13)
AGAACCGCGCGGGCCAATCGCGCTGCTCCCGGGTGATGACGTAGGCTGCGCCTGTGCATGCGCAGGGAGG
GGAGACCTTGGCGGAGCGGCGGAGGCGCCCAGCGGAGGTGAAAGTATTGGCGGAAAGGAAAATACAGCGG
AAAAATGCAGAGCTGGAGTCGTGTGTACTGCTCCTTGGCCAAGAGAGGCCATTTCAATCGAATATCTCAT
GGCCTACAGGGACTTTCTGCAGTGCCTCTGAGAACTTACGCAGATCAGCCGATTGATGCTGATGTAACAG
TTATAGGTTCTGGTCCTGGAGGATATGTTGCTGCTATTAAAGCTGCCCAGTTAGGCTTCAAGACAGTCTG
CATTGAGAAAAATGAAACACTTGGTGGAACATGCTTGAATGTTGGTTGTATTCCTTCTAAGGCTTTATTG
AACAACTCTCATTATTACCATATGGCCCATGGAAAAGATTTTGCATCTAGAGGAATTGAAATGTCCGAAG
TTCGCTTGAATTTAGACAAGATGATGGAGCAGAAGAGTACTGCAGTAAAAGCTTTAACAGGTGGAATTGC
CCACTTATTCAAACAGAATAAGATAGATGAAGATACAATAGTGTCATCTACAGGTGCTTTATCTTTAAAA
AAAGTTCCAGAAAAGATGGTTGTTATTGGTGCAGGAGTAATAGGTGTAGAATTGGGTTCAGTTTGGCAAA
GACTTGGTGCAGATGTGACAGCAGTTGAATTTTTAGGTCATGTAGGTGGAGTTGGAATTGATATGGAGAT
ATCTAAAAACTTTCAACGCATCCTTCAAAAACAGGGGTTTAAATTTAAATTGAATACAAAGGTTACTGGT
GCTACCAAGAAGTCAGATGGAAAAATTGATGTTTCTATTGAAGCTGCTTCTGGTGGTAAAGCTGAAGTTA
TCACTTGTGATGTACTCTTGGTTTGCATTGGCCGACGACCCTTTACTAAGAATTTGGGACTAGAAGAGCT
GGGAATTGAACTAGATCCCAGAGGTAGAATTCCAGTCAATACCAGATTTCAAACTAAAATTCCAAATATC
TATGCCATTGGTGATGTAGTTGCTGGTCCAATGCTGGCTCACAAAGCAGAGGATGAAGGCATTATCTGTG
TTGAAGGAATGGCTGGTGGTGCTGTGCACATTGACTACAATTGTGTGCCATCAGTGATTTACACACACCC
TGAAGTTGCTTGGGTTGGCAAATCAGAAGAGCAGTTGAAAGAAGAGGGTATTGAGTACAAAGTTGGGAAA
TTCCCATTTGCTGCTAACAGCAGAGCTAAGACAAATGCTGACACAGATGGCATGGTGAAGATCCTTGGGC
AGAAATCGACAGACAGAGTACTGGGAGCACATATTCTTGGACCAGGTGCTGGAGAAATGGTAAATGAAGC
TGCTCTTGCTTTGGAATATGGAGCATCCTGTGAAGATATAGCTAGAGTCTGTCATGCACATCCGACCTTA
TCAGAAGCTTTTAGAGAAGCAAATCTTGCTGCGTCATTTGGCAAATCAATCAACTTTTGAATTAGAAGAT
TATATATATTTTTTTCTGAAATTTCCTGGGAGCTTTTGTAGAAGTCACATTCCTGAACAGGATATTCTCA
CAGCTCCAAGAATTTCTAGGACTGAATTATGAAACTTTTGGAAGGTATTTAATAGGTTTGGACAAAATGG
AATACTCTTATATCTATATTTTACATAAATTTAGTATTTTGTTTCAGTGCACTAATGTGTAAGACAAAAA
GCTACTTATTGTAGCATCCTGGAATATCTCCGTCAACTCATATTTTCATGCTGTTCATGAAAGATTCAAT
GCCCCTGAATTTAAATAGCTTTTTTCTCTGATACAGAAAAGTTGAATTTTACATGGCTGGAGCTAGAATT
TGATATGTGAACAGTTGTGTTTGAAGCACAGTGATCAAGTTATTTTTAATTTGGTTTTCACATTGGAAAC
AAGTCAGTCATTCAGATATGATTCAAATGTCTATAAACCGAACTGATGTAAGTAAACGGTCTCTCACTTG
TTTTATTTAACCTCTAAATTCTTTCATTTTAGGGGTAGCATTTGTGTTGAAGAGGTTTTAAAGCTTCCAT
TGTTGTCTGCAACTCTGAAGGGTAATTATATAGTTACCCAAATTAAGAGAGTCTATTTACGGAACTCAAA
TACGTGGGCATTCAAATGTATTACAGTGGGGAATGAAGATACTGAAATAAACGTCTTAAATATTCATTTA
CTGGTTATCATGAGTACGTGTTGAGATGGTCATAGTTTTTTTTATGACTACTTCTAGTGTATATTCTAAT
TTCTTTTCTAGGCCTGAATGTATCTTTATTTTCATGTTATAGGACAATATTAAGGCATTTTAAAGGTCAT
CATCCTTTCATCTATTTTAGATACACCTACTAAATGTTTAATATATACTTTTGGAGAAGTACAACATAAG
GGAGTCTTTAATCTGTGTTTTCCTTGGCTGGGTTAATGACTGTTTATTTAAAGAGTGTTGTAAAATTGGA
TGTGTGGTGTTTAAAATGGCCATGTCCTGAGGAAACTTAAGTAACAAAGTACTAAATGCTAAGTAGGCTT
TTGCATATTGTAACTAAATTTAAGAATAATTCAGATTAAGTAGTTCTGAAATTTGGTATAGATAGCATAG
ATTGTCTCATGCTCATGAGTGACATAATGACCCTGGATTCTGTTACATACTTCTAAAGAAAATTGATTGT
TGTCTTAGGAGGCAGTTAACTTGGCTGAACACCAACTCCACACTCTGTCTTGTTTGTAGGTGGCAGCAGC
TGAAATCTCTTCTCAGTTGTTTTAGCTTTAGCTATGCTGCTGGAAGTCTTTCCCATGCAAGTGTGTAGTT
CAGGGGTCAACCAGAGTTTGGGCAGAAGGAAGTCTGCCCCTTCTGTGCCTCCTGTTTTTTGGGGGTTTCC
CCTTTATGTTCCAGCTGTTGTGGTTGCCCCATATTCTGCCTTCTGATCCTTAACCAATAAAACTTGGCTT
TTGTTTCCCCCTCAAGTGAGAACCCGTTAAAAATGAGACATTGAGCCAGTGCTGTTCACTTTTTAAGTGC
CAACTTCCCTCTACTTTCCACTTGTTTATAGTTGTTTCCAGTGCCTTTAGTTTTTTCTAAAATATATTTG
TTCAGAGTTTGCAGTTGCTATCAGCAGGAGGGTTGGTCTGATATCTGTGTGCTACTTTGCCATTATTGGA
AGTGAACTCTGCATCTTTTTAAAAATTTGAAATCCCGGTATCATGTGAAGTGCTGTTTATGTAAATCTCA
ACATATCCCTTACTCAGGGAAAAAAAAGTTTTTAGTTAGGGAATAGTGAAATATAATTTAATATGGAATT
CTAGCTGTAGAGTTAAATCCATCTTTAAGTGTTTACATTCAGTATGAGAATGCAAATTTATCTGTATGGG
GAATAAAGTCCTAGGAATAAAACAAGTTTTAAGTGTTCA

In one aspect, the present disclosure provides OGDH-specific, DLST-specific or DLD-specific inhibitory nucleic acids comprising a nucleic acid molecule, which is complementary to a portion of an OGDH, DLST or DLD nucleic acid sequence selected from the group consisting of SEQ ID NOs: 4-13.

The present disclosure also provides an antisense nucleic acid comprising a nucleic acid sequence that is complementary to and specifically hybridizes with a portion of any one of SEQ ID NOs: 4-13 (OGDH, DLST or DLD mRNA), thereby reducing or inhibiting expression of OGDH, DLST or DLD. The antisense nucleic acid may be antisense RNA, or antisense DNA. Antisense nucleic acids based on the known OGDH, DLST or DLD gene sequence can be readily designed and engineered using methods known in the art.

Antisense nucleic acids are molecules which are complementary to a sense nucleic acid strand, e.g., complementary to the coding strand of a double-stranded DNA molecule (or cDNA) or complementary to an mRNA sequence. Accordingly, an antisense nucleic acid can form hydrogen bonds with a sense nucleic acid. The antisense nucleic acid can be complementary to an entire OGDH, DLST or DLD coding strand, or to a portion thereof, e.g., all or part of the protein coding region (or open reading frame). In some embodiments, the antisense nucleic acid is an oligonucleotide which is complementary to only a portion of the mRNA coding region of OGDH, DLST or DLD. In certain embodiments, an antisense nucleic acid molecule can be complementary to a noncoding region of the OGDH coding strand, DLST coding strand or DLD coding strand. In some embodiments, the noncoding region refers to the 5′ and 3′ untranslated regions that flank the coding region and are not translated into amino acids. For example, the antisense oligonucleotide can be complementary to the region surrounding the translation start site of OGDH, DLST or DLD. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual β-units, the strands run parallel to each other (Gaultier et al., Nucleic Acids. Res. 15:6625-6641(1987)). The antisense nucleic acid molecule can also comprise a 2′-O-methylribonucleotide (Inoue et al., Nucleic Acids Res. 15:6131-6148 (1987)) or a chimeric RNA-DNA analogue (Inoue et al., FEBS Lett. 215:327-330 (1987)).

The present disclosure also provides a short hairpin RNA (shRNA) or small interfering RNA (siRNA) comprising a nucleic acid sequence that is complementary to and specifically hybridizes with a portion of any one of SEQ ID NOs: 4-13 (mRNA of OGDH, DLST or DLD), thereby reducing or inhibiting expression of OGDH, DLST or DLD. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hybridize to a mRNA of a target gene. Following intracellular delivery, the siRNA or shRNA molecule associates with an RNA-induced silencing complex (RISC), which then binds and degrades a complementary target mRNA (such as mRNA of OGDH, DLST or DLD).

The present disclosure also provides a ribozyme comprising a nucleic acid sequence that is complementary to and specifically hybridizes with a portion of any one of SEQ ID NOs: 4-13 (OGDH, DLST or DLD mRNA), thereby reducing or inhibiting expression of OGDH, DLST or DLD. Ribozymes are catalytic RNA molecules with ribonuclease activity which are capable of cleaving a complementary single-stranded nucleic acid, such as an mRNA. Thus, ribozymes (e.g., hammerhead ribozymes (described in Haselhoff and Gerlach, Nature 334:585-591 (1988))) can be used to catalytically cleave OGDH, DLST or DLD transcripts, thereby inhibiting translation of OGDH, DLST or DLD.

A ribozyme having specificity for a nucleic acid encoding OGDH, DLST or DLD can be designed based upon a nucleic acid sequence of OGDH, DLST or DLD. For example, a derivative of a Tetrahymena L-19 IVS RNA can be constructed in which the nucleotide sequence of the active site is complementary to the nucleotide sequence to be cleaved in a mRNA encoding OGDH, DLST or DLD. See, e.g., U.S. Pat. Nos. 4,987,071 and 5,116,742. Alternatively, mRNA of a OGDH, DLST or DLD can be used to select a catalytic RNA having a specific ribonuclease activity from a pool of RNA molecules. See, e.g., Bartel and Szostak (1993) Science 261:1411-1418, incorporated herein by reference.

The present disclosure also provides a synthetic guide RNA (sgRNA) comprising a nucleic acid sequence that is complementary to and specifically hybridizes with a portion of any one of SEQ ID NOs: 4-13 (mRNA of OGDH, DLST or DLD). Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of any one of SEQ ID NOs: 4-13, and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465.

In some embodiments, a synthetic guide RNA is a single RNA represented as comprising the following elements:

5′-X1-X2-Y-Z-3′

where X1 and X2 represent the crRNA segment, where X1 is the targeting sequence that binds to a portion of any one of SEQ ID NOs: 4-13, X2 is a stem sequence the hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of any one of SEQ ID NOs: 4-13, X2 is a stem sequence the hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator:

(a)
(SEQ ID NO: 14)
NNNNNNNNNNNNNNNNNNNNgtttttgtactctcaagatttaGAAAtaaa
tcttgcagaagctacaaagataaggcttcatgccgaaatcaacaccctgt
cattttatggcagggtgttttcgttatttaaTTTTTT;
(b)
(SEQ ID NO: 15)
NNNNNNNNNNNNNNNNNNNNgtttttgtactctcaGAAAtgcagaagcta
caaagataaggcttcatgccgaaatcaacaccctgtcattttatggcagg
gtgttttcgttatttaaTTTTTT;
(c)
(SEQ ID NO: 16)
NNNNNNNNNNNNNNNNNNNNgtttttgtactctcaGAAAtgcagaagcta
caaagataaggcttcatgccgaaatcaacaccctgtcattttatggcagg
gtgtTTTTTT;
(d)
(SEQ ID NO: 17)
NNNNNNNNNNNNNNNNNNNNgttttagagctaGAAAtagcaagttaaaat
aaggctagtccgttatcaacttgaaaaagtggcaccgagtcggtgcTTTT
TT;
(e)
(SEQ ID NO: 18)
NNNNNNNNNNNNNNNNNNNNgttttagagctaGAAATAGcaagttaaaat
aaggctagtccgttatcaacttgaaaaagtgTTTTTTT;
and
(f)
(SEQ ID NO: 19)
NNNNNNNNNNNNNNNNNNNNgttttagagctagAAATAGcaagttaaaat
aaggctagtccgttatcaTTTTTTTT

Selection of suitable oligonucleotides for use in as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site. In some embodiments, the CRISPR enzyme is a nickase, which cleaves only one strand of the target nucleic acid.

### Pharmaceutical Compositions

In one aspect, the present disclosure provides pharmaceutical compositions comprising alpha-ketoglutarate (αKG), glutamine, succinate (e.g., dimethyl-succinate), an OGDH inhibitor, DLST inhibitor and/or DLD inhibitor as described herein.

The pharmaceutical compositions of the present disclosure may be prepared by any of the methods known in the pharmaceutical arts. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, the amount of active compound will be in the range of about 0.1 to 99 percent, more typically, about 5 to 70 percent, and more typically, about 10 to 30 percent.

In some embodiments, pharmaceutical compositions of the present technology may contain one or more pharmaceutically-acceptable carriers, which as used herein, generally refers to a pharmaceutically-acceptable composition, such as a liquid or solid filler, diluent, excipient, manufacturing aid (e.g., lubricant, talc magnesium, calcium or zinc stearate, or steric acid), or solvent encapsulating material, useful for introducing the active agent into the body.

Examples of suitable aqueous and non-aqueous carriers that may be employed in the pharmaceutical compositions of the present technology include, for example, water, ethanol, polyols (such as glycerol, propylene glycol, polyethylene glycol, and the like), vegetable oils (such as olive oil), and injectable organic esters (such as ethyl oleate), and suitable mixtures thereof. Proper fluidity can be maintained, for example, by the use of coating materials, such as lecithin, by the maintenance of the required particle size in the case of dispersions, and by the use of surfactants.

In some embodiments, the formulations may include one or more of sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; alginic acid; buffering agents, such as magnesium hydroxide and aluminum hydroxide; pyrogen-free water; isotonic saline; Ringer's solution; ethyl alcohol; pH buffered solutions; polyesters, polycarbonates and/or polyanhydrides; preservatives; glidants; fillers; and other non-toxic compatible substances employed in pharmaceutical formulations.

Various auxiliary agents, such as wetting agents, emulsifiers, lubricants (e.g., sodium lauryl sulfate and magnesium stearate), coloring agents, release agents, coating agents, sweetening agents, flavoring agents, preservative agents, and antioxidants can also be included in the pharmaceutical composition of the present technology. Some examples of pharmaceutically-acceptable antioxidants include: water soluble antioxidants, such as ascorbic acid, cysteine hydrochloride, sodium bisulfate, sodium metabisulfite, sodium sulfite, and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. In some embodiments, the pharmaceutical formulation includes an excipient selected from, for example, celluloses, liposomes, micelle-forming agents (e.g., bile acids), and polymeric carriers, e.g., polyesters and polyanhydrides. Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Prevention of the action of microorganisms on the active compounds may be ensured by the inclusion of various antibacterial and antifungal agents, such as, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption, such as aluminum monostearate and gelatin.

In some embodiments, the alpha-ketoglutarate (αKG), glutamine, succinate (e.g., dimethyl-succinate), an OGDH inhibitor, DLST inhibitor and/or DLD inhibitor as described herein can be targeted for delivery to the intestinal epithelium by applying an outer polymeric film coat with pH-dependent solubility/permeability, or possibly, by using a drug-containing matrix with suitable swelling/solubility characteristics, i.e. delayed release. In some embodiments, the pH for solubility of the polymer is slightly below 6.0. A threshold pH—for solubility of the polymer—slightly below 6.0 is generally considered to be sufficient to achieve protection from release in the stomach followed by a fast onset of release in the small intestine, e.g. polymers such as hydroxypropyl methylcellulose phthalate (HPMCP 55) and the 1:1 co-polymer of methacrylic acid and ethyl acrylate (Eudragit L100-55, L30D-55) which are soluble above pH 5.5, will usually dissolve immediately after the dosage form has been emptied from the stomach. Gastroresistant polymers with a threshold pH for solubility up to 7.0 are commercially available. Eudragit L (co-polymer of methacrylic acid and methylmethacrylate 1:1), with a threshold pH for solubility of 6.0, displayed lag times in vivo for dissolution of 3-5 h when applied in relatively large amounts on thus indicating release in the distal parts of the small intestine and proximal large intestine. When applied on to pellets (1.7-2.0 mm) in a relatively thin film (30 pm), the disintegration time was approximately 2 h, indicating release in the mid-small intestine.

In some embodiments, the alpha-ketoglutarate (αKG), glutamine, succinate (e.g., dimethyl-succinate), an OGDH inhibitor, DLST inhibitor and/or DLD inhibitor as described herein can be targeted for delivery to the intestinal epithelium using time-triggered systems, or gradient-triggered systems. Examples of time-triggered systems include, the Time Clock system, the Pulsincap system and Oros CT. The Time Clock system consists of a layer of a hydrophobic material (e.g. carnauba wax, white beeswax), a hydrophilic polymer (hydroxypropyl methylcellulose) and a surfactant (Tween 80) coated on to a fast-disintegrating tablet core. The Pulsincap consists of a water insoluble capsule with a hydrogel cap (crosslinked polyethylene glycol). The Oros CT consists of small enteric-coated tablets in a hard gelatin capsule.

A classical concept for spatial delivery to the large intestine is to utilize the expected rise in pH along the intestine. Several 5-aminosalicylic acid formulations are commercially available which utilize pH-sensitive coatings to target the distal small intestine and/or the large intestine. A relatively thick tablet coating of a polymer with a threshold pH for solubility of 6.0 was shown to disintegrate mostly in the small intestine (70%) or the proximal colon. A coating with a higher pH threshold for solubility, e.g. the 1:2 co-polymer of methacrylic acid and methyl methacrylate (Eudragit S, pH7) is also used for commercial preparations. Other suitable coating ingredients include 5-aminosalicylic acid prodrugs, film-forming polymers, dietary fibres, polysaccharides, inulin, xanthan gum, locust bean gum, portulaca-derived polysaccharides, pectin and guar gum.

Polyacid polymers, such as alginate and hyaluronic acid, are intrinsically suited for delivery in the gut; at an acidic pH, they can be found in their neutral shrunken form, protecting the drug during the passage through the stomach. In contrast, when the pH is higher than the acid dissociation constant (pKa), they assume an ionized form, which promotes electrostatic repulsion among the polymer chains and gel swelling. Carboxymethyl starch (CM) is a pH-responsive excipient used as a drug gastro-protector due to the presence of carboxyl groups on its starch chains. Cholesteryl hemisuccinate (CHEMS) is widely used to provide liposomes with pH sensitivity because of its lipophilicity and membrane stabilization activity. Layered double hydroxides (LDHs) are cationic layered compounds with exchangeable anions between the layers, which enable the drug interlayer loading and pH-responsive release. Another pH-dependent release strategy includes functionalization with listeriolysin O (LLO) or silica mesoporous microparticles or coating nanoparticles with calixarenes.

In some embodiments, the alpha-ketoglutarate (αKG), glutamine, succinate (e.g., dimethyl-succinate), an OGDH inhibitor, DLST inhibitor and/or DLD inhibitor as described herein can be targeted for delivery to the intestinal epithelium using nanocarriers. The nanocarriers may be inorganic or organic nanocarriers. Examples of suitable nanocarriers include extracellular vesicles, lipid-based nanoparticles, microspheres, liposomes, inorganic nanoparticles, SPIONs, SiNPs, and polymeric nanoparticles. In certain embodiments, liposomes may be modified with molecules, such as PEG+functionalizing layer, antibodies with cleavable sites, carbohydrates, and hydrophobic/hydrophilic drugs.

Inorganic nanocarriers may be modified with antibodies and/or drugs. In particular, drugs can be contained inside (such as in microspheres) or linked outside (such as in SiNPs) the carrier. Moreover, modifications can adhere to the surface of the nanoparticles by means of surfactants or they could be packaged in the pores (such as in the case of multistage vectors).

An option for hydrophobic/lipophilic drug delivery through oral administration is represented by self-emulsifying drug delivery systems (SEDDS), also known as SNEDDS or SMEDDS to indicate their nano (N) or micro (M) size. SEDDS consist of mixtures of oils, co/surfactants, and co/solvents that form isotropic blends and are often administered through gelatin capsules. SEDDS/SNEDDS/SMEDDS systems can improve the bioavailability of drugs and protect them from precipitation and premature degradation.

In some embodiments, the alpha-ketoglutarate (αKG), glutamine, succinate (e.g., dimethyl-succinate), an OGDH inhibitor, DLST inhibitor and/or DLD inhibitor as described herein are released into the intestinal epithelium by degradation due to colonic flora or intestinal enzymes, or external stimuli-driven release (e.g., ultrasound, heat, light, magnetic fields, internal radiation, or electrical and mechanical triggers).

In some embodiments, the alpha-ketoglutarate (αKG), glutamine, succinate (e.g., dimethyl-succinate), an OGDH inhibitor, DLST inhibitor and/or DLD inhibitor as described herein can be targeted for delivery to the intestinal epithelium by targeting receptors overexpressed in affected cells. This is achieved by functionalizing nanocarriers with ligands (e.g., receptor-specific antibodies, HA, mannose, AS1411, EGF and GE11, Urotensin, Folic acid, VATANST peptide, FA12 peptide and AR13 peptide). Additional strategies for targeting delivery of alpha-ketoglutarate (αKG), glutamine, succinate (e.g., dimethyl-succinate), an OGDH inhibitor, DLST inhibitor and/or DLD inhibitor into intestinal epithelium are described in Garbati et al., Pharmaceutics 2024, 16, 431, which are incorporated by reference herein.

### Therapeutic and Prophylactic Methods

The following discussion is presented by way of example only, and is not intended to be limiting.

One aspect of the present technology includes methods of treating a gastrointestinal inflammatory disease (e.g., colitis or Chron's disease) characterized by elevated expression levels and/or increased activity of OGDH complex.

In one aspect, the present disclosure provides a method for treating gastrointestinal inflammatory disease (e.g., colitis or Chron's disease) in a subject in need thereof, comprising administering to the subject a therapeutically effective amount of at least one agent that increases alpha-ketoglutarate (αKG) activity, wherein the subject suffers from a disease or condition characterized by elevated expression levels and/or increased activity of OGDH complex.

In some embodiments, the subject is diagnosed as having, suspected as having, or at risk of having a disease or condition characterized by elevated expression levels and/or increased activity of OGDH complex. Additionally or alternatively, in some embodiments, the subject is diagnosed as having gastrointestinal inflammatory disease (e.g., colitis or Chron's disease).

In therapeutic applications, compositions or medicaments comprising at least one agent that increases alpha-ketoglutarate (αKG) activity as disclosed herein are administered to a subject suspected of, or already suffering from such a disease or condition (such as, a subject diagnosed with a disease or condition characterized by elevated expression levels and/or increased activity of OGDH complex and/or a subject diagnosed with gastrointestinal inflammatory disease (e.g., colitis or Chron's disease), in an amount sufficient to cure, or at least partially arrest, the symptoms of the disease, including its complications and intermediate pathological phenotypes in development of the disease.

Subjects suffering from a disease or condition characterized by elevated expression levels and/or increased activity of OGDH complex and/or a subject diagnosed with gastrointestinal inflammatory disease (e.g., colitis or Chron's disease) can be identified by any or a combination of diagnostic or prognostic assays known in the art.

In some embodiments, subjects with a disease or condition characterized by elevated expression levels and/or increased activity of OGDH complex, and/or subjects suffering from gastrointestinal inflammatory disease (e.g., colitis or Chron's disease) that are treated with the at least one agent that increases alpha-ketoglutarate (αKG) activity will show amelioration or elimination of one or more of the following symptoms: diarrhea, fatigue, abdominal pain and cramping, bloody stool, reduced appetite, unintended weight loss, and rectal bleeding.

In certain embodiments, subjects with a disease or condition characterized by elevated expression levels and/or increased activity levels of OGDH complex, and/or subjects suffering from gastrointestinal inflammatory disease (e.g., colitis or Chron's disease) that are treated with the at least one agent that increases alpha-ketoglutarate (αKG) activity will show enhanced tissue regeneration, and/or increased abundance of secretory intestinal cells compared to untreated subjects suffering from the same disease or condition.

In one aspect, the present technology provides a method for preventing or delaying the onset of a disease or condition characterized by elevated expression levels and/or increased activity of OGDH complex. Additionally or alternatively, in some aspects, the present technology provides a method for preventing or delaying the onset of gastrointestinal inflammatory disease (e.g., colitis or Chron's disease).

Subjects at risk or susceptible to a disease or condition characterized by elevated expression levels and/or increased activity of OGDH complex, and/or subjects at risk or susceptible to gastrointestinal inflammatory disease (e.g., colitis or Chron's disease). Such subjects can be identified by, e.g., any or a combination of diagnostic or prognostic assays known in the art.

In prophylactic applications, pharmaceutical compositions or medicaments comprising at least one agent that increases alpha-ketoglutarate (αKG) activity as disclosed herein are administered to a subject susceptible to, or otherwise at risk of a disease or condition characterized by (a) elevated expression levels and/or increased activity of OGDH complex, and/or (b) a subject susceptible to, or otherwise at risk of gastrointestinal inflammatory disease (e.g., colitis or Chron's disease), in an amount sufficient to eliminate or reduce the risk, or delay the onset of the disease, including biochemical, histologic and/or behavioral symptoms of the disease, its complications and intermediate pathological phenotypes presenting during development of the disease. Administration of a prophylactic agent that increases alpha-ketoglutarate (αKG) activity can occur prior to the manifestation of symptoms characteristic of the disease or disorder, such that the disease or disorder is prevented or, alternatively, delayed in its progression.

In some embodiments, treatment with the at least one agent that increases alpha-ketoglutarate (αKG) activity will prevent or delay the onset of one or more of the symptoms of gastrointestinal inflammatory disease (e.g., colitis or Chron's disease) such as diarrhea, fatigue, abdominal pain and cramping, bloody stool, reduced appetite, unintended weight loss, and rectal bleeding.

In one aspect, the present disclosure provides a method for treating or preventing a gastrointestinal inflammatory disease in a subject in need thereof comprising administering to the subject a therapeutically effective amount of an agent that increases alpha-ketoglutarate (αKG) expression and/or activity. In another aspect, the present disclosure provides a method for selecting a subject suffering from a gastrointestinal inflammatory disease for treatment with an agent that increases alpha-ketoglutarate (αKG) activity comprising detecting OGDH mRNA and/or OGDH polypeptide levels in a biological sample obtained from the patient that are elevated relative to a predetermined threshold or a reference sample obtained from a healthy control subject; and administering to the subject an effective amount of an agent that increases αKG activity. Additionally or alternatively, in some embodiments, the expression levels of OGDH are detected via RT-PCR, Northern Blotting, RNA-Seq, microarray analysis, High-performance liquid chromatography (HPLC), mass spectrometry, immunohistochemistry (IHC), fluorescence in situ hybridization (FISH), Western Blotting, immunoprecipitation, flow cytometry, Immuno-electron microscopy, immunoelectrophoresis, enzyme-linked immunosorbent assays (ELISA), or multiplex ELISA antibody arrays.

In any and all embodiments of the methods disclosed herein, the gastrointestinal inflammatory disease is colitis or Chron's disease.

Additionally or alternatively, in some embodiments, the agent is αKG, glutamine, succinate, or dimethyl-succinate. In any of the preceding embodiments of the methods disclosed herein, the agent inhibits expression and/or activity of a component of oxoglutarate dehydrogenase (OGDH) complex. The components of OGDH complex include oxoglutarate dehydrogenase (OGDH), dihydrolipoyl succinyltransferase (DLST), or dihydrolipoyl dehydrogenase (DLD). In some embodiments, the agent is a small molecule, an inhibitory nucleic acid that specifically targets the component of OGDH complex, or a neutralizing antibody that specifically targets the component of OGDH complex. Examples of small molecule OGDH inhibitors include, but are not limited to succinyl phosphonate, (S)-2-[(2,6-dichlorobenzoyl)amino]succinic acid (AA6), KGD09, KGD02, and derivatives (e.g., azide-alkyne cyclized derivatives of KGD09, and KGD02) thereof. Examples of small molecule DLST inhibitors include, but are not limited to obatoclax mesylate, CP724714, LY317615 (enzastaurin), gemcitabine, BMS 708163 (Avagacestat), Lapatinib, or IPA3. Examples of small molecule DLD inhibitors include, but are not limited to carmustine, lomustine, 5-methoxyindole-2-carboxylic acid (MICA), valproic acid (VPA) derivatives such as valproyl-CoA and valproyl-dephospho-CoA. In some embodiments, the inhibitory nucleic acid that specifically targets the component of OGDH complex is a siRNA, a shRNA, an antisense oligonucleotide, or a sgRNA. The inhibitory nucleic acid that specifically targets the component of OGDH complex may be operably linked to a heterologous promoter, an inducible promoter, a constitutive promoter, a tissue-specific promoter, or an ubiquitous promoter. In some embodiments, the inhibitory nucleic acid that specifically targets the component of OGDH complex is selectively expressed in intestinal epithelial cells.

In any of the preceding embodiments, the agent is administered orally, topically, intranasally, systemically, intravenously, subcutaneously, intraperitoneally, intradermally, intraocularly, iontophoretically, transmucosally, or intramuscularly.

Additionally or alternatively, in some embodiments, the methods of the present technology further comprise separately, sequentially or simultaneously administering one or more additional therapeutic agents to the subject. Examples of additional therapeutic agents that are useful for treating or preventing gastrointestinal inflammatory diseases include, but are not limited to, aminosalicylates (e.g., sulfasalazine, mesalamine, olsalazine, balsalazide), corticosteroids (e.g., Prednisone, Prednisolone, Methylprednisolone, Budesonide), azathioprine, 6-mercaptopurine, cyclosporine, tacrolimus, ozanimod, tofacitinib, upadacitinib, etrasimod, adalimumab, golimumab, infliximab, ustekinumab and vedolizumab.

In any and all embodiments of the methods disclosed herein, administration of the agent that increases αKG activity results in an increased abundance of secretory intestinal cells in the subject. Additionally or alternatively, in some embodiments, administration of the agent does not alter biosynthetic function or viability of enterocytes in the subject.

In one aspect, the present disclosure provides a method for enhancing tissue regeneration in a subject that has received radiation therapy comprising administering to the subject a therapeutically effective amount of alpha-ketoglutarate (αKG) or glutamine. In some embodiments, the radiation therapy is external beam radiation therapy. The tissue regeneration may comprise regeneration of intestinal tissue or colon tissue.

For therapeutic and/or prophylactic applications, a composition comprising alpha-ketoglutarate (αKG), glutamine, succinate (e.g., dimethyl-succinate), an OGDH inhibitor, a DLST inhibitor or DLD inhibitor disclosed herein, is administered to the subject. In some embodiments, alpha-ketoglutarate (αKG), glutamine, succinate (e.g., dimethyl-succinate), the OGDH inhibitor, the DLST inhibitor or DLD inhibitor is administered one, two, three, four, or five times per day. In some embodiments, alpha-ketoglutarate (αKG), glutamine, succinate (e.g., dimethyl-succinate), the OGDH inhibitor, the DLST inhibitor or DLD inhibitor is administered more than five times per day. Additionally or alternatively, in some embodiments, alpha-ketoglutarate (αKG), glutamine, succinate (e.g., dimethyl-succinate), the OGDH inhibitor, the DLST inhibitor or DLD inhibitor is administered every day, every other day, every third day, every fourth day, every fifth day, or every sixth day. In some embodiments, alpha-ketoglutarate (αKG), glutamine, succinate (e.g., dimethyl-succinate), the OGDH inhibitor, the DLST inhibitor or DLD inhibitor is administered weekly, bi-weekly, tri-weekly, or monthly. In some embodiments, alpha-ketoglutarate (αKG), glutamine, succinate (e.g., dimethyl-succinate), the OGDH inhibitor, the DLST inhibitor or DLD inhibitor is administered for a period of one, two, three, four, or five weeks. In some embodiments, alpha-ketoglutarate (αKG), glutamine, succinate (e.g., dimethyl-succinate), the OGDH inhibitor, the DLST inhibitor or DLD inhibitor is administered for six weeks or more. In some embodiments, alpha-ketoglutarate (αKG), glutamine, succinate (e.g., dimethyl-succinate), the OGDH inhibitor, the DLST inhibitor or DLD inhibitor is administered for twelve weeks or more. In some embodiments, alpha-ketoglutarate (αKG), glutamine, succinate (e.g., dimethyl-succinate), the OGDH inhibitor, the DLST inhibitor or DLD inhibitor is administered for a period of less than one year. In some embodiments, alpha-ketoglutarate (αKG), glutamine, succinate (e.g., dimethyl-succinate), the OGDH inhibitor, the DLST inhibitor or DLD inhibitor is administered for a period of more than one year. In some embodiments, alpha-ketoglutarate (αKG), glutamine, succinate (e.g., dimethyl-succinate), the OGDH inhibitor, the DLST inhibitor or DLD inhibitor is administered throughout the subject's life.

In some embodiments of the methods of the present technology, alpha-ketoglutarate (αKG), glutamine, succinate (e.g., dimethyl-succinate), the OGDH inhibitor, the DLST inhibitor or DLD inhibitor is administered daily for 1 week or more. In some embodiments of the methods of the present technology, alpha-ketoglutarate (αKG), glutamine, succinate (e.g., dimethyl-succinate), the OGDH inhibitor, the DLST inhibitor or DLD inhibitor is administered daily for 2 weeks or more. In some embodiments of the methods of the present technology, alpha-ketoglutarate (αKG), glutamine, succinate (e.g., dimethyl-succinate), the OGDH inhibitor, the DLST inhibitor or DLD inhibitor is administered daily for 3 weeks or more. In some embodiments of the methods of the present technology, alpha-ketoglutarate (αKG), glutamine, succinate (e.g., dimethyl-succinate), the OGDH inhibitor, the DLST inhibitor or DLD inhibitor is administered daily for 4 weeks or more. In some embodiments of the methods of the present technology, alpha-ketoglutarate (αKG), glutamine, succinate (e.g., dimethyl-succinate), the OGDH inhibitor, the DLST inhibitor or DLD inhibitor is administered daily for 6 weeks or more. In some embodiments of the methods of the present technology, alpha-ketoglutarate (αKG), glutamine, succinate (e.g., dimethyl-succinate), the OGDH inhibitor, the DLST inhibitor or DLD inhibitor is administered daily for 12 weeks or more. In some embodiments, alpha-ketoglutarate (αKG), glutamine, succinate (e.g., dimethyl-succinate), the OGDH inhibitor, the DLST inhibitor or DLD inhibitor is administered daily throughout the subject's life.

### Determination of the Biological Effect of the Therapeutic Agents of the Present Technology

In various embodiments, suitable in vitro or in vivo assays are performed to determine the effect of alpha-ketoglutarate (αKG), glutamine, succinate (e.g., dimethyl-succinate), or a specific OGDH inhibitor, DLST inhibitor or DLD inhibitor and whether its administration is indicated for treatment. In various embodiments, in vitro assays can be performed with representative animal models, to determine if alpha-ketoglutarate (αKG), glutamine, succinate (e.g., dimethyl-succinate), or a given OGDH inhibitor, DLST inhibitor or DLD inhibitor exerts the desired effect on reducing or eliminating signs and/or symptoms of gastrointestinal inflammatory disease (e.g., colitis or Chron's disease). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects. In some embodiments, in vitro or in vivo testing is directed to the biological function of one or more of alpha-ketoglutarate (αKG), glutamine, succinate (e.g., dimethyl-succinate), or a specific OGDH inhibitor, DLST inhibitor or DLD inhibitor. In some embodiments, in vitro or in vivo testing is directed to the biological function of DLD protein, DLST protein and/or OGDH protein.

Animal models of gastrointestinal inflammatory diseases may be generated using techniques known in the art (see, e.g., Examples described herein). Such models may be used to demonstrate the biological effect of alpha-ketoglutarate (αKG), glutamine, succinate (e.g., dimethyl-succinate), an OGDH inhibitor, a DLST inhibitor or a DLD inhibitor disclosed herein in the prevention and treatment of gastrointestinal inflammatory diseases, and for determining what comprises a therapeutically effective amount of the one or more of alpha-ketoglutarate (αKG), glutamine, succinate (e.g., dimethyl-succinate), OGDH inhibitors, DLST inhibitors or DLD inhibitors disclosed herein in a given context.

### Modes of Administration and Effective Dosages

Any method known to those in the art for contacting a cell, organ or tissue with one or more of alpha-ketoglutarate (αKG), glutamine, succinate (e.g., dimethyl-succinate), OGDH inhibitors, DLST inhibitors, or DLD inhibitors disclosed herein may be employed. Suitable methods include in vitro, ex vivo, or in vivo methods. In vivo methods typically include the administration of one or more of alpha-ketoglutarate (αKG), glutamine, succinate (e.g., dimethyl-succinate), OGDH inhibitors, DLST inhibitors, or DLD inhibitors to a mammal, suitably a human. When used in vivo for therapy, the one or more of alpha-ketoglutarate (αKG), glutamine, succinate (e.g., dimethyl-succinate), OGDH inhibitors, DLST inhibitors, or DLD inhibitors described herein are administered to the subject in effective amounts (i.e., amounts that have desired therapeutic effect). The dose and dosage regimen will depend upon the degree of the disease state of the subject, the characteristics of alpha-ketoglutarate (αKG), glutamine, succinate (e.g., dimethyl-succinate) or the particular OGDH inhibitor, DLST inhibitor or DLD inhibitor used, e.g., its therapeutic index, and the subject's history.

The effective amount may be determined during pre-clinical trials and clinical trials by methods familiar to physicians and clinicians. An effective amount of one or more of alpha-ketoglutarate (αKG), glutamine, succinate (e.g., dimethyl-succinate), OGDH inhibitors, DLST inhibitors, or DLD inhibitors useful in the methods may be administered to a mammal in need thereof by any of a number of well-known methods for administering pharmaceutical compounds. The alpha-ketoglutarate (αKG), glutamine, succinate (e.g., dimethyl-succinate), OGDH inhibitor, DLST inhibitor, or DLD inhibitor may be administered systemically or locally.

One or more of alpha-ketoglutarate (αKG), glutamine, succinate (e.g., dimethyl-succinate), the OGDH inhibitors, the DLST inhibitors, or the DLD inhibitors described herein can be incorporated into pharmaceutical compositions for administration, singly or in combination, to a subject for the treatment or prevention of gastrointestinal inflammatory disease (e.g., colitis or Chron's disease). Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more of alpha-ketoglutarate (αKG), glutamine, succinate (e.g., dimethyl-succinate), the OGDH inhibitors, the DLST inhibitors, or the DLD inhibitors disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomersal, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more of alpha-ketoglutarate (αKG), glutamine, succinate (e.g., dimethyl-succinate), OGDH inhibitors, DLST inhibitors, or DLD inhibitors disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more of alpha-ketoglutarate (αKG), glutamine, succinate (e.g., dimethyl-succinate), OGDH inhibitor, DLST inhibitor, or DLD inhibitor concentrations in a carrier may range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more of alpha-ketoglutarate (αKG), glutamine, succinate (e.g., dimethyl-succinate), the OGDH inhibitors, the DLST inhibitors, or the DLD inhibitors disclosed herein may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In some embodiments, one or more of alpha-ketoglutarate (αKG), glutamine, succinate (e.g., dimethyl-succinate), OGDH inhibitors, DLST inhibitors, and/or DLD inhibitors disclosed herein may be combined with one or more additional therapies for the prevention or treatment of gastrointestinal inflammatory disease (e.g., colitis or Chron's disease). Additional therapeutic agents include, but are not limited to, aminosalicylates (e.g., sulfasalazine, mesalamine, olsalazine, balsalazide), corticosteroids (e.g., prednisone, prednisolone, methylprednisolone, budesonide), immunomodulators (e.g., azathioprine, 6-mercaptopurine, cyclosporine, tacrolimus), ozanimod, tofacitinib, upadacitinib, etrasimod, adalimumab, certolizumab pegol, natalizumab, infliximab-dyyb, risankizumab-rzaa, golimumab, infliximab, ustekinumab, vedolizumab, mirikizumab-mrkz, antibiotics (e.g., Metronidazole, ampicillin, ciprofloxacin), etc.

Additionally or alternatively, in some embodiments, one or more of alpha-ketoglutarate (αKG), glutamine, succinate (e.g., dimethyl-succinate), OGDH inhibitors, DLST inhibitors, and/or DLD inhibitors disclosed herein may be separately, sequentially or simultaneously administered with at least one additional therapeutic agent selected from the group consisting of aminosalicylates (e.g., sulfasalazine, mesalamine, olsalazine, balsalazide), corticosteroids (e.g., prednisone, prednisolone, methylprednisolone, budesonide), immunomodulators (e.g., azathioprine, 6-mercaptopurine, cyclosporine, tacrolimus), ozanimod, tofacitinib, upadacitinib, etrasimod, adalimumab, certolizumab pegol, natalizumab, infliximab-dyyb, risankizumab-rzaa, golimumab, infliximab, ustekinumab, vedolizumab, mirikizumab-mrkz, and antibiotics (e.g., Metronidazole, ampicillin, ciprofloxacin).

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits comprising alpha-ketoglutarate (αKG), glutamine, succinate (e.g., dimethyl-succinate), an OGDH inhibitor, a DLST inhibitor and/or a DLD inhibitor disclosed herein. Optionally, the above described components of the kits of the present technology are packed in suitable containers and labeled for the prevention and/or treatment of gastrointestinal inflammatory disease (e.g., colitis or Chron's disease) or enhancing tissue regeneration in a subject that has received radiation therapy.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

The present technology is further illustrated by the following Examples, which should not be construed as limiting in any way.

### Example 1: Materials and Methods

### Mouse Models

Housing conditions: All animal experiments in this study were performed in accordance with protocols approved by the Memorial Sloan Kettering Institutional Animal Care and Use Committee (approval number: 11-06-012). The mice were housed with a 12 h light/dark cycle between 8:00 and 20:00 in a temperature-controlled room (22±1° C.) with free access to water and food.

Generation of inducible Ogdh knockdown mouse: Considering that the ODGH knockout is embryonic lethal⁴¹, we developed an inducible model utilizing doxycycline (Dox)-inducible short hairpin RNAs (shRNA) linked to a GFP reporter. This system enables temporal and reversible suppression of OGDH expression and facilitates the tracing and analysis of cells exhibiting OGDH knockdown using the GFP reporter. To account for potential off-target effects of RNA interference (RNAi), we used two validated shRNAs (shOgdh.2081 and shOgdh.346)¹. As a control for non-sequence-based effects of perturbing the RNAi machinery, we used a similar construct harboring a Renilla luciferase-targeting shRNA (shRen_731), which does not target any gene expressed in mouse cells. Dox-inducible GFP-coupled shRNA constructs were electroporated and integrated into a “homing cassette” at the Co1A1 locus in 4482 embryonic stem cells (ESCs)^{29,32}, which contains a Dox-inducible reverse tet-transactivator (rtTA-M2) expressed from the Cag-rtTA promoter. Validated clones were utilized to generate chimeric mice using 8-cell aggregation, allowing assessment of functionality in F0. The resulting founders were backcrossed to establish germline transmission, generating Tg. TRE-shOgdh.2081, Tg. TRE-shOgdh.346, and Tg. TRE-shRenilla.731 mice. To further amplify Ogdh knockdown and enable widespread expression of the TRE-GFP-shRNA cassette, including in the intestine 32, we crossed TRE-shRenilla and TRE-shOgdh mice with CAGs-rtTA3 transgenic animals, resulting in TRE-shRenilla^{Cag-rtTA3} and TRE-shOgdh^{Cag-rtTA3} mice.

ISC analysis: To enable isolation of Lgr5⁺ cells for assessing TCA cycle enzyme expression, we used Lgr5-EGFP-IRES-creERT2 mice²⁷.

### Mouse Diets and Treatments

Cag-rtTA3 mediating shRenilla and shOgdh expression was activated by feeding mice with doxycycline hyclate diet (200 mg/kg) at adult stage, changed twice per week.

Acute DSS treatment was performed as previously described⁴². Briefly, at 10 weeks of age, mice were treated with 2% DSS (MP-Biomedicals, 021160110-CF) in drinking water for 5 days. Afterwards mice were transferred to regular drinking water and sacrificed at then indicated time points. In all experiments with DSS models, mice were weighed at the beginning of DSS treatment and every other day thereafter. In addition, they were evaluated daily for signs of distress or end-point criteria. Specifically, mice were immediately euthanized if lost more than 20% of initial body weight or showed breathing difficulties.

For Bromodeoxyuridine (BrdU) pulse mice were injected with 1 mg/mouse of BrdU (Sigma-Aldrich) in PBS and sacrificed 2 hrs later.

For DM-αKG supplementation, mice were injected (intraperitoneally) once daily with 600 mg/kg or 300 mg/kg of DM-αKG (349631-5G, Sigma-Aldrich) dissolved in PBS. For the vehicle control, mice were injected with PBS.

For pulsatile inhibition of Ogdh, mice were injected intraperitoneally with doxycycline (2.5 mg/kg, Sigma Aldrich, D9891) once daily in cycles of 3 consecutive days ON followed by 4 days OFF.

### Genotyping PCR

Genomic DNA was extracted from mouse tails and ear punches. Biopsies were digested in MGB buffer supplemented with 10% TritonX, 1% BME and 0.4 mg/ml of proteinase K (Qiagen, 19133) and incubated overnight at 55° C. PCR conditions: 95° C.-6 min, 35× cycle (95° C.-40 sec, 62° C.-45 sec, 72° C.-1 min), 72° C.-10 min. Primers: SApA-F 1-5′-CTGCTGTCCATTCCTTATTC-3′ (SEQ ID NO: 1), Rev 2-5′-CGAAACTCTGGTTGACATG-3 (SEQ ID NO: 2), For 1-5′-TGCCTATCATGTTGTCAAA-3 (SEQ ID) NO: 3).

RNA-Seq Analysis and qPCR

RNA extraction, RNA-seq library preparation and sequencing: Total RNA was isolated from crypts isolated from TRE-shRenilla^{Cag-rtTA3}, TRE-shOgdh^{Cag-rtTA3}, vehicle-treated, or DM-αKG-treated mice using RNeasy kits (Qiagen, 74004). RNA concentration and quality was assessed using an Agilent 2100 Bioanalyzer. Sequencing and library preparation was performed at the Integrated Genomics Operation at Memorial Sloan Kettering Cancer Center (MSKCC). RNA-seq libraries were prepared from total RNA. After RiboGreen quantification and quality control by Agilent BioAnalyzer, 100-500 ng of total RNA underwent polyA selection and TruSeq library preparation according to instructions provided by Illumina (TruSeq Stranded mRNA LT Kit, RS-122-2102), with 8 cycles of PCR. Samples were barcoded and run on a HiSeq 4000 or HiSeq 2500 in a 50 bp/50 bp paired end run, using the HiSeq 3000/4000 SBS Kit or TruSeq SBS Kit v4 (Illumina) at MSKCC's Integrated Genomics Operation. An average of 41 million paired-end reads was generated per sample. Ribosomal reads represented at most 0.01% of the total reads generated, and the fraction of mRNA averaged 53%. RNA-seq read mapping, differential expression analysis and heatmap visualization: Adaptor sequences were removed from the RNA-seq data using Trimmomatic⁴³. The trimmed reads were then aligned to the GRCm38.91 (mm10) reference genome using STAR⁴⁴, and transcript count was quantified using featureCounts⁴⁵ to generate a raw count matrix. Differential gene expression analysis was conducted using the DESeq2 package⁴⁶ in R (http://cran.r-project.org/), comparing the experimental conditions. Each condition had 3-5 independent biological replicates (individual mice). Principal component analysis (PCA) was performed using DESeq2 to visualize the variation in gene expression among the samples. Differentially expressed genes (DEGs) were identified based on a >2-fold change in gene expression with an adjusted P-value <0.05. To visualize the DEGs, the samples were z-score normalized and plotted as a heatmap using the ‘pheatmap’ package in R. Functional annotations of the gene sets were conducted through pathway enrichment analysis using the Reactome, Azimut, CellType, and KEGG databases. The analysis was performed with enrichR⁴⁷, and the significance of the tests was assessed using a combined score, calculated as log(p)*z, where p represents the p-value from the Fisher exact test, and z is the z-score indicating the deviation from the expected rank. Gene set enrichment analysis (GSEA)² was conducted using the GSEA-Preranked tool (version 2.07). The analysis involved the enrichment of gene sets using RNA-seq data obtained from the experiment. The gene sets were derived from the MSigDB database (http://software.broadinstitute.org/gsea/msigdb), as well as signatures previously published from mouse intestine

qRT-PCR

For quantitative RT-PCR analysis, total RNA was extracted from mESCs, isolated crypts or sorted cells Lgr5-EGFP mouse using the RNeasy Mini Kit (Qiagen, 74004). Subsequently, cDNA synthesis was carried out using TaqMan reverse transcription reagents (Applied Biosystems). Real-time PCR was performed in triplicate using SYBR Green PCR Master Mix (Applied Biosystems) on the ViiA 7 Real-Time PCR System (Invitrogen). The expression levels of target genes were normalized to endogenous control genes, 3B64 and β-Actin. Gene-specific primer sets were designed using the qPrimerDepot tool provided by the National Center for Biotechnology Information (www.ncbi.nlm.nih.gov/tools/primer-blast/) or published elsewhere.

### Intestine Preparation

For immunofluorescence analysis: small intestine and colon were removed from mice and flushed with PBS. Subsequently, intestines were opened longitudinally, “swiss-rolled,” incubated overnight in 10% formalin at room temperature, changed to 70% EtOH and processed for paraffin embedding.

For crypt isolation: freshly isolated small intestines from C57Bl/6, TRE-shOgdh^{Cag-rtTA3} and TRE-shRen^{Cag-rtTA3} were harvested and longitudinally opened through the lumen. Specifically, the duodenum (first 10-15 cm from the stomach) was collected, as it yields more crypts in a shorter time. The collected duodenum was washed in ice-cold HBSS and kept in ice-cold HBSS until the rest of the samples were harvested. Next, the intestine was transferred to a 15-ml Falcon tube, then chopped into pieces of 1-2 cm. For each tube, 6-7 ml of ice-cold 8 mM EDTA in HBSS was added, and the tube was incubated on ice for 15 minutes. After incubation, the EDTA solution was removed and replaced with HBSS. The tube was then vigorously shaken for approximately 20 seconds. A 20-1 aliquot was taken from the supernatant and examined under a microscope to confirm that it contains the villi fraction. The supernatant was discarded, and the intestine pieces were then transferred to a new 15 ml Falcon tube and rinsed in ice-cold HBSS to remove any residual villi. Another 6-7 ml of ice-cold 8 mM EDTA in HBSS was added per tube, followed by a 15-minute incubation on ice. Like the previous step, the EDTA solution was removed and replaced with HBSS (optional step), and the tube was shaken vigorously for approximately 20 seconds. A 20 μl aliquot of the supernatant was taken and examined under the microscope to check whether it contained the desired crypt fraction. Depending on the yield, these steps were repeated until the crypts were enriched in the supernatant.

### Culture of Intestinal Organoids

Regular conditions: freshly isolated crypts were embedded in 50 μl of undiluted Matrigel (356231; Cultek) and cultured in DMEM/F12 media (D8437; Sigma-Aldrich) supplemented with Pen/Strep, 1×(2 mM) Glutamax (35050038; Gibco or Life Technologies/Thermo Fisher Scientific), 10 mM Hepes (15630049; Gibco or Life Technologies/Thermo Fisher Scientific), 2 mM N-acetyl cysteine (A8199-10G; Sigma-Aldrich), 1×B27 supplement (17504-044; Life Technologies), 10 mM nicotinamide (N0636-100G; Sigma-Aldrich), 50 ng/ml recombinant mEGF (PMG8044; Gibco), 100 ng/ml recombinant Noggin (250-38; PeproTech), 1 μg/ml murine R-spondin 1 (120-38; PeproTech), and 1% Normocin (ant-nr-1; InvivoGen)⁴⁸. During the initial 12-hour culture establishment, 1.5 μM CHIR99021 (GSK3 inhibitor; 2520691; PeproTech) and 10 μM Y-27632 inhibitor (1293823; PeproTech) were included, and then the medium was replaced. The medium was changed every other day.

Organoid differentiation: Organoid differentiation assays were performed as previously described²⁸. Briefly, crypts or single cells were entrapped in Matrigel and plated at the center of wells in a 24-well plate. Following polymerization of Matrigel, 500 μl of complete Advanced DMEM/F12 was added. Enrichment media were as follows:

ISC enrichment media (ENR-CV): EGF (50 ng/ml; Life Technologies), Noggin (100 ng/ml; PeproTech), R-spondin 1 (500 ng/ml; R & D), and small molecules including CHIR99021 (3 μM; Stemgent) and valproic acid (1 mM; Sigma-Aldrich).

Paneth cell enrichment media (ENR-CD): EGF (50 ng/ml; Life Technologies), Noggin (100 ng/ml; PeproTech), R-spondin 1 (500 ng/ml; R & D), and small molecules including CHIR99021 (3 μM; Stemgent) and DAPT (10 μM; Sigma-Aldrich).

Goblet cell enrichment media (ENR-VD): EGF (50 ng/ml; Life Technologies), Noggin (100 ng/ml; PeproTech), R-spondin 1 (500 ng/ml; R & D), and small molecules including valproic acid (1 mM; Sigma-Aldrich) and DAPT (10 μM; Sigma-Aldrich).

Enterocyte enrichment media (ENR-IV): EGF (50 ng/ml; Life Technologies), Noggin (100 ng/ml; PeproTech), R-spondin 1 (500 ng/ml; R & D), and small molecules including valproic acid (1 mM; Sigma-Aldrich) and IWP2 (2 mm/Sigma Aldrich)

To enrich in progenitor cells, organoids were kept in ENR-CV for 6 days and then transferred for 3 days to the corresponding differentiation media. For fully mature lineages, ISC-enriched organoids were maintained in differentiation media for at least 6 days.

### Metabolomics

Intestinal organoid culture: Intestinal organoid culture was performed by isolating crypts through mechanical disruption with EDTA, followed by embedding them in Matrigel. To ensure consistent cell numbers, crypts from five independent mice were pooled together, and the resulting pool was embedded in Matrigel. Five Matrigel drops (30-40 ul per drop) were plated in every 6-well plate. Triplicate samples were plated from the pooled crypts to maintain consistency in the experiments, as the LC-MS is highly sensitive to cell number variations, and it is challenging to plate the same number of organoids from different mice. Organoids were kept in the pertinent culture media as described above. Also, as internal control for the metabolites presents in Matrigel, in LC-MS experiments, we isolated the metabolites present in Matrigel (same number of drops) without organoids but going through the same culture protocol.

Sample preparation: supernatant was aspirated, then the intracellular metabolites were extracted by adding 1 mL ice-cold 80% methanol directly to the Matrigel drops with organoids and incubated overnight at −80° C. to aid protein precipitation. The following day, the methanol extracts were centrifuged at 20,000×g for 20 min at 4° C., and 800 μL of the supernatant was transferred to a new tube and evaporated in a vacuum concentrator (GeneVac). The samples were reconstituted in 80 μL of 40% acetonitrile+20 μL 100% methanol or 100 μL of 50% methanol and incubated on ice for 20 min, vortexing every 5 min to ensure adequate re-suspension. All samples underwent one final centrifugation (20,000×g for 20 min at 4° C.) to remove any residual particulate. As control for potential metabolites in the Matrigel, we also extracted metabolites from Matrigel alone subjected to the same treatments as the organoids (250 μl, approximately).

LC-MS/MS analysis: For metabolomic profiling using liquid chromatography-mass spectrometry, dried extracts were resuspended in 80 μL of 40% acetonitrile in water+20 μL 100% methanol for hydrophilic interaction liquid chromatography (HILIC) or in 100 μL of 50% methanol in water for ion pair liquid chromatography separations. Samples were vortexed, incubated on ice for 20 min, and clarified by centrifugation at 20,000g for 20 min at 4° C.

HILIC LC-MS analysis was performed on a 6545 Q-TOF mass spectrometer (Agilent Technologies) in positive ionization mode. Liquid chromatography separation was achieved on a ACQUITY UPLC BEH Amide column (150 mm×2.1 mm, 1.7 m particle size, Waters) using a gradient of solvent A (10 mM ammonium acetate in 10:90 acetonitrile:water with 0.2% acetic acid, pH 4) and solvent B (10 mM ammonium acetate in 90:10 acetonitrile:water with 0.2% acetic acid, pH 4). The gradient was 0 min, 95% B; 9 min, 70% B; 13 min, 30% B; 14 min, 30% B; 14.5 min, 95% B; 15 min, 95% B; and 20 min, 95% B. Other LC parameters were as follows: flow rate 400 μL/min, column temperature 40° C., and injection volume 5 μl. Other MS parameters were as follows: gas temperature 300° C.; gas flow 10 l/min; nebulizer pressure 35 psi; sheath gas temperature 350° C.; sheath gas flow 12 l/min; VCap 4,000 V; and fragmentor 125 V.

Ion pair LC-MS analysis was performed on a 6230 TOF mass spectrometer (Agilent Technologies) in negative ionization mode, liquid chromatography separation was achieved on a XSelect HSS T3 column (150 mm×2.1 mm, 3.5 m particle size, Waters) using a gradient of solvent A (5 mM octylamine in water with 5 mM acetic acid) and solvent B (5 mM octylamine in methanol with 5 mM acetic acid) and post column solvent with 90:10 acetone:DMSO. The gradient was at 0.3 mL/min: 0 min, 1% B; 3.5 min, 1% B; 4 min, 35% B; 15 min, 35% B; 20 min, 100% B; at 0.4 mL/min 20.10 min, 100% B; and 22 min, 100% B, 22.10 min, 1% B; and 27 min, 1% B. Other LC parameters were as follows: post column flow rate 0.3 mL/min, column temperature 40° C., and injection volume 5 μl. Other MS parameters were as follows: gas temp: 250° C.; gas flow: 9 L/min; nebulizer pressure: 35 psi; sheath gas temp: 250° C.; sheath gas flow: 121/min; VCap: 3,500 V; and fragmentor: 125 V.

Targeted data analysis was performed using both Skyline and MassHunter Profinder software v.10.0 (Agilent Technologies). Additional analysis was done using Metaboanalyst (www.metaboanalyst.ca/MetaboAnalyst/ModuleView.xhtml).

### Seahorse Assay in Organoids

Crypt extraction: Crypts were extracted as previously described. Organoid preparation for Seahorse experiments: freshly isolated crypts were resuspended in 1.5 ml of Matrigel. Then, 1, 2, or 3 μl of Matrigel with organoids were plated in the center of a 96-well Seahorse plate, positioned between the three dots. It is important to note that Matrigel polymerizes quickly, so it should be kept on ice during the plating process. To ensure consistent conditions, in control wells, the same volumes of Matrigel were plated. The optimal number of organoids to plate can vary depending on the system, so different concentrations were tried initially. To account for variations in plating the same number of organoids in each well, an entire column per condition was plated to have eight replicates. Appropriate organoid culture media was added to each well, and the plate was kept in the incubator until the day of the experiment. Typically, the organoids were allowed to adapt for 2 days before the experiment was performed. Seahorse experiment: the assay was performed as previously described with some adaptations⁴⁹. Briefly, the day before the experiment, Complete Seahorse media was prepared. This media was formulated using Agilent Seahorse XF (pH=7.4) without phenol red and supplemented with glucose (10 mM), pyruvate (1 mM), and glutamine (2 mM). All reagents were adjusted to a pH of 7.4. The sensor plate was activated in water overnight at 37° C. On the day of the experiment, the organoids were prepared by removing the organoid complete media and washing twice with Complete Seahorse media. The Complete Seahorse media was added to the organoids, and they were allowed to adapt for 30 minutes (maximum 1 hour) at 37° C. in a non-C02 incubator. Injections and settings: For the injection solutions, the following concentrations were used: Oligomycin (port A) at 50 μM, FCCP at 20 M, and Rotenone and Antimycin A at 20 μM each. The settings of the XF Analyzer for the assay were as follows: Basal (3 cycles) with a mix time of 4 minutes, a wait time of 0 minutes, and a measure time of 3 minutes; Oligomycin (6 cycles) with a mix time of 4 minutes, a wait time of 0 minutes, and a measure time of 3 minutes; FCCP (3 cycles) with a mix time of 4 minutes, a wait time of 0 minutes, and a measure time of 3 minutes; and Rotenone and Antimycin A (3 cycles) with a mix time of 4 minutes, a wait time of 0 minutes, and a measure time of 3 minutes. Normalization: Take a 4×BF picture from all wells to check organoid positioning after the assay. For normalization, count number of organoids per well that are located between the 3 dots. This normalization works well for conditions with similar size/viability. If there are differences in organoid, % of OCR, directly provided by Agilent software (https://seahorseanalytics.agilent.com) was used.

Preparation of Single Cell Suspensions from Intestinal and Colonic Mucosa for Cell Soring and FACS-Based Immunophenotyping

Intestinal preparation: crypts were isolated as described above. After spinning, crypts were incubated in DMEM/F12 medium (D8437; Sigma-Aldrich) supplemented with 0.8 U/ml dispase (17105041; Life Technologies/Thermo Fisher Scientific) and 1 mg/ml DNase (04716728001; Roche). Cells were incubated in 2 ml of “digestion solution” for 10-15 minutes at 37° C., vortexing the samples every 2 minutes for 30 seconds. After 10 minutes, 20 ul sample was taken and observed under the microscope to check single cell dissociation. Digestion was stop by adding 10 ml of FBS. Subsequently, cells were filtered through a 70-μM mesh, spin down at 1,200 rpm for 5 min, and resuspended in MACS buffer (0.5% BSA and 2 mM EDTA in Ca²⁺/Mg²⁺-free PBS). GFP^{high} (ISCs), GFP^{low} (TA cells), and GFP⁻ high side scattering/forward scattering (Paneth) and villi fraction (1^{st} fraction during mechanical cell extraction) were isolated from Lgr5-EGFP-IRES-creERT2 and sorted using Sony MA900 cell sorter. Colon preparation: for immunophenotyping of the lamina propria and the muscle layer in the colon of control and DSS-treated mice, samples were prepared as previously reported⁵⁰. Briefly, the entire colon was harvested, opened longitudinally, and washed in 1×HBSS (w/o). Colon was chopped into pieces of 0.5-1 cm and crypts were mechanically dissected using 8 mM EDTA. Remaining colon pieces were transferred to digestion mix with Dispase and DNAse. Immune cells were further sedimented by centrifugation and immunophenotyping analysis were performed as described below.

### FACS-Based Immunophenotyping

For multi-parametric flow cytometry analysis, cell suspensions were stained with LIVE/DEAD fixable viability dye (1:500, Invitrogen, R37601) for 30 min in PBS at 4° C. After this, cells were washed, incubated with Fc block (1:200, BD Bioscience, 564219) in FACS buffer for 15 min at 4° C., and then stained with a cocktail of conjugated antibodies (See below) for 30 min on ice. After staining, cells were washed 3 times with FACS buffer and fixed using BD Cytofix/Cytoperm (Fisher Scientific, 544772) for 20 min at 4° C., washed again, and stored for analysis. Samples were analyzed in a BD LSR Fortessa with 5 lasers, where gates were set by use of fluorescence-minus-one (FMO) controls.

The following antibodies were used to quantify the fraction and identity of IL1RL1 ST2 receptor expressing cells: AF700 CD45 (BioLegend, 103128, Clone 30-F11), BUV395 CD11b (BD Biosciences, 563553, Clone M1/70), BV785 CD11c (BioLegend, 117335 Clone N418), PE F4/80 (BioLegend, 123110, Clone BM8), BV605 Ly6G (BD Bioscience, 563005, Clone 1A8), APC Cy7 Ly6c (BioLegend, 128026, Clone HK1.4), APC MHCII (BioLegend, 107614, Clone M5/114.15.2), BV710 CD206 (BioLegend, 141727, Clone C068C2), BV650 CD86 (BioLegend, 105035, GL-1).

### Immunofluorescence

Mouse tissues: Mouse tissues were fixed overnight at 4° C. in 10% formalin before paraffin-embedding. 5-micrometer sections were deparaffinized and rehydrated with histoclear and alcohol series and subjected to antigen retrieval by boiling in citrate antigen retrieval buffer (Vector). Slides were blocked in PBS with 5% BSA, and primary antibody staining was performed in blocking buffer+0.02% Triton overnight at 4° C. The following primary antibodies were used: chicken anti-GFP (1:500, Abcam 13970), mouse anti-Ki67 (1:500, BD, 550609), rabbit anti-p53 (1:500, NCL-L-p53-CM5p, Leica Biosystems), rabbit anti-5hmC (1:500, Active Motif, 39769), mouse anti-β-catenin (1:200, BD, 610153), rabbit anti Ogdh (1:100, Proteintech, 15212-1-AP), rabbit anti VDAC (1:100, Abcam, ab15895), goat anti Ace2 (1:100, Thermo Scientific, PA5-47488), rabbit anti Lysozyme (1:500, Thermo Scientific, MA5-32154), rabbit anti BrdU (1:100, Abeam, ab6326), rabbit anti Cl. Caspase 3 (1:200 Cell Signaling, 9664S), mouse anti Hnf4α (1:100, Thermo Scientific, MA1-199), mouse anti Tet1 (1:100, Thermo Scientific, MA5-16312), rabbit anti Tet2 (1:100, Thermo Scientific, PA5-85488), rabbit anti Tet3, (1:100, Thermo Scientific, PA5-31860), rat anti Cd8 (1:200, 14-0808-82, Thermo Scientific) rat anti Cd4 (1:100, Thermo Scientific, 14-9766-82). Primary antibodies were detected with the following fluorescently conjugated secondary antibodies: goat-anti-chicken AF488 (Life Technologies A-11039), goat anti-rabbit AF488 (Life Technologies A-32723), goat anti-rabbit AF594 (Life Technologies A-11037), goat anti-mouse AF488 (Life Technologies, A-32723), goat anti-mouse A F594 (Life Technologies, A-11032), goat anti-rat AUF488 (Life Technologies, A-11006) and goat anti-rat 594 (Life Technologies, A-11007). All secondary antibodies were diluted in blocking buffer+0.02% Triton and incubated for 1 h at room temperature. Subsequently, slides were washed with PBS and nuclei were counterstained with PBS containing DAPI and mounted under cover slips with ProLong Gold (LifeTechnologies).

Human samples: samples used in human studies were obtained from Tissue Array (www.tissuearray.com/). Specifically, TMAs C0809b, C0246, CO245a were utilized. Immunofluorescence staining procedures were carried out as previously described. Same primary and secondary antibodies were used.

Organoid immunofluorescence: organoids were recovered using Cell Recovery Solution (354253; Corning BD) and fixed with 4% paraformaldehyde for 20 min at room temperature (18-21° C.). Next, the samples were passed through an ethanol series (70, 96, and 100%) and embedded in paraffin. Immunohistochemistry and immunofluorescence were performed using standard techniques as previously described. Same primary and secondary antibodies were used.

Image acquisition and analysis: images were acquired with a Zeiss AxioImager microscope using Axiovision software. 5-10 images per slide were obtained. Quantification was performed either by counting the number of positive cells per crypt or villi (at least 50 crypts and 100 villi were quantified) or the percentage of the positive area using Color Deconvolution plug-in in ImageJ v1.7 software. Different macros were also developed by the authors to quantify immunofluorescence images.

Single Molecule Fluorescence In Situ Hybridization (smFISH) Collection

smFISH analysis were performed as previously described⁵¹.

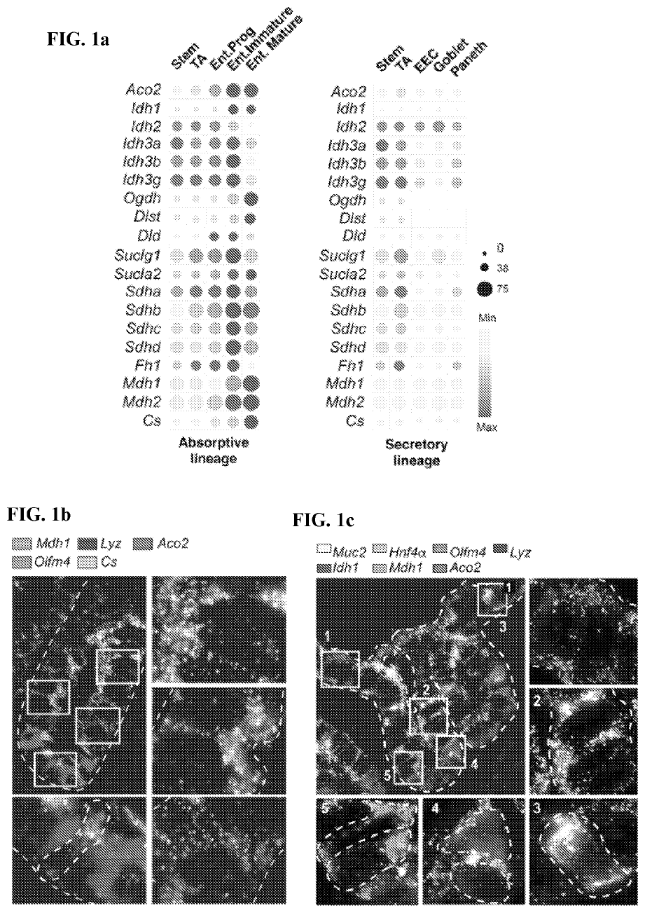

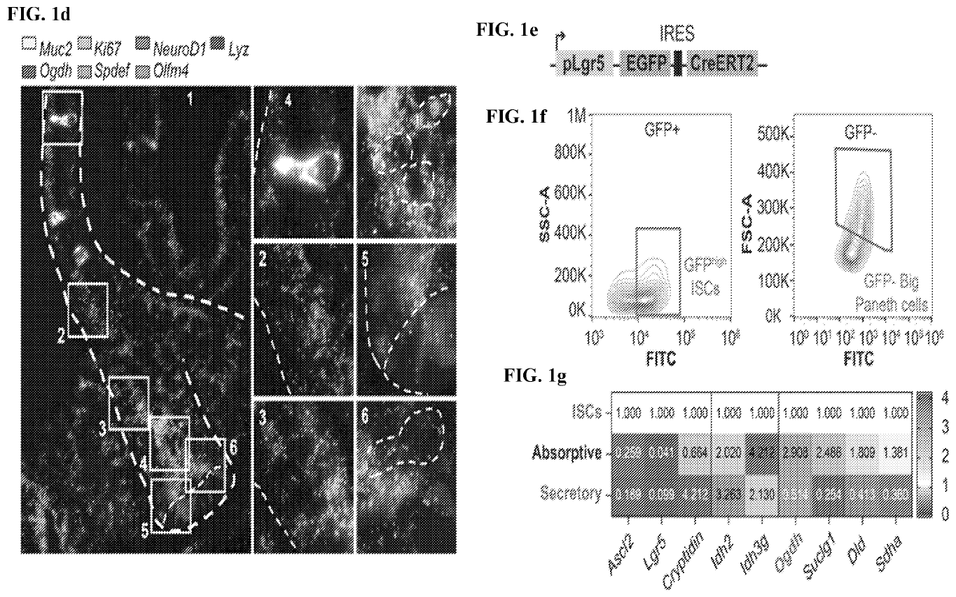

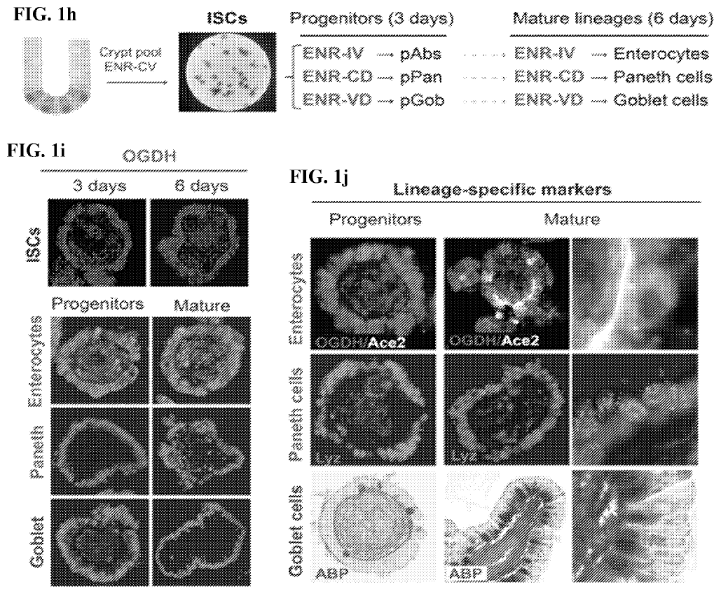

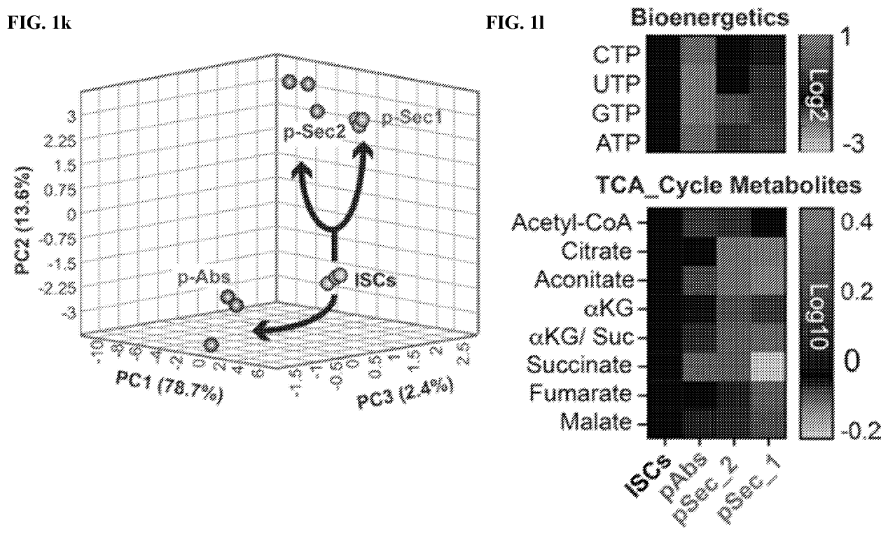

Probe design for multiplex single molecule FISH: We built upon published software^{52,53} to design custom panels for single molecule mRNA FISH. This design strategy relies on pre-computation of all possible 30-mer sequences found in mouse cDNAs (Ensembl GRCm38.p6), augmented with coding sequences of fluorescent proteins engineered into our mouse model. We excluded pseudogenes from the potential pool of mRNAs to design probes for. We compute multiple scores for each 30-mer, including Tm, GC content, and potential for hybridization with rRNAs and tRNAs. We used the following parameters to include a 30 mer into our candidate probe-set: GC-content (43-63%), Tm (66-76° C.), excluding 30-mers that contain at least a 15 merpresent in a rRNA or tRNA. In addition, we computed expression-informed penalties to estimate the specificity of each candidate probe. We adapted published software^{52,53} to include single-cell information into the estimation of specificities scores. We reasoned that incorporating single-cell information would decrease the chances of selecting probes with off-target binding to highly expressed genesin rare cell populations. To do so, we leveraged published single-cell data used in FIG. 1²⁴. Following suggested parameters from the original MERFISH publications^{52,53}, we considered 30-mers with a specificity score greater than 0.75 as candidates for our panels. We aimed to select 92 non-overlapping probes per gene. Whenever this wasn't possible due to transcript length, homology to other genes, or other sequence properties, we allowed a maximum overlap of 20 bp between probes.

Sample preparation for multiplex single molecule FISH: “Swiss roll” intestines were positioned in a cassette and fixed with 4% PFA 1×PBS solution for 4 hrs at 4° C. Cassettes were then transferred to 4% PFA 30% sucrose 1×PBS, and incubated overnight at 4° C. To preserve villi structures, cassettes were transferred for 4 hrs to 30% sucrose/50% OCT for 3 hrs at RT and finally embedded in Tissue Plus O.C.T. Compound (Fisher Healthcare, 4585) in a cryomold (Tissue-Tek, 4557). Molds were placed on dry ice until all O.C.T. was frozen and intestinal samples were stored at −80° C.

Coverslip preparation for smFISH staining: Coverslips for smFISH staining were prepared as previously described⁵¹. Briefly, 40 mm-diameter (Bioptechs, 0420-0323-2) were cleaned by immersing them in a 1:1 mix of 37% HCl and methanol at room temperature for 30 min. Coverslips were then washed with Milli-Q water, and one time with 70% ethanol, followed by gently drying with nitrogen gas. Cleaned coverslips were submerged in 0.1% (vol/vol) triethylamine (Millipore, TX1200) and 0.2% (vol/vol) allyltrichlorosilane (Sigma, 107778) in chloroform for 30 min at RT. They were washed once with chloroform, once with 100% ethanol and dried up using nitrogen gas. Coverslips were stored long-term in a desiccated chamber at RT.

Poly-lysine coating of coverslips: To prepare coverslips for intestinal staining individual samples, pre-treated coverslips were coated with 0.1 mg/mL Poly-D lysine (Thermo Scientific, A3890401) for 1 hr at RT. Next, they were washed 1 time with 1×PBS, and 3 times with nuclease-free water. After that, they were left to dry for at least 2 hrs before sectioning the tissue.

Tissue sectioning, fixation and permeabilization for smFISH staining: Tissue sections of 10 m thickness were mounted into poly-D lysine coated coverslips. Coverslips were dried for 5-10 min at 50° C. and placed on dry ice until completion of sectioning of all samples. Next, plates with coverslips were transferred to ice, and treated with 3 mL 1×PBS followed by fixation at room temperature with 4% PFA 1×PBS for 10 min. Coverslips were then washed three times with 1×PBS and maintained at 4° C. overnight in ice-cold 70% ethanol for permeabilization.

Pre-staining treatment of permeabilized tissues: After overnight incubation, coverslips were rehydrated with 1×PBS on ice for 10 min. To bleach endogenous fluorescence of lineage reporters and reduced autofluorescence from lysozyme granules, tissues were incubated with 3% hydrogen peroxide (Fisher, H325-500), 1:600 37% HCl (vol/vol) 1×PBS and placed under a heat lamp for 1 hr at room temperature. They were then washed 2 times with 1×PBS, and one time with 2×SSC. Next, they were treated digestion solution (pre-warmed at 37° C.) containing a final concentration of 20 g/mL proteinase K (Sigma, 3115836001), 2×SSC solution, and incubated at 37° C. for 10 min. Next, coverslips were washed 3 times with 2×SSC and treated with pre-hybridization solution (30% formamide (Thermo Scientific, AM9344), 2×SSC) and for at least 3 h at 37° C.

Staining solution with primary probes: Primary probes were diluted at a 100 nM final concentration per probe in 3H staining buffer, (30% formamide, 10% dextran sulfate (Sigma Aldrich, D8906-50G), 1 mg/mL yeast tRNA (Thermo Fisher Scientific, 15401029) and 2×SSC). In addition, 2 M anchor probe was added to the staining solution containing specific probes. Upon completion of pre-hybridization incubation, coverslips were incubated with 100 μL droplet of hybridization solution+probes (100 nM per probe) and they were placed on a 15 cm dish, with a wet Kimwipe used as a humidity buffer, and incubated at 37° C. for 36 h-48 h.

Post-hybridization wash: post-hybridization wash buffer (30% formamide and 2×SSC) was pre-heated to 37° C. Coverslips were washed twice with post-hybridization wash buffer at 47° C. for 30 min. Lastly, coverslips were transferred to 2×SSC solution and maintained at 4° C. until the next step.

Gel embedding: Samples were embedded on a thin layer of polyacrylamide gel, to allow subsequent tissue-clearing through digestion of protein and lipids. The gel solution was composed of 4% (vol/vol) of 19:1 acrylamide/bis-acrylamide (BioRad, 1610144), 60 mM Tris·HCl pH 8 (Invitrogen, 15568-025), 0.3 M NaCl (Boston Bioproducts, R-244), supplemented with the polymerizing agents ammonium persulfate (Sigma, 09913) and TEMED (Sigma, T7024) at final concentrations of 0.03% (wt/vol) and 0.15% (vol/vol), respectively, and polymerized as previously described⁵¹. Polymerization completed in the course 2 h at room temperature. After, gel-embedded coverslips were transferred to a 6 cm tissue culture dish with 2×SSC.

Digestion: Gel embedded samples were treated overnight at 37° C. with digestion solution (2% SDS (Invitrogen, AM9822), 0.25% TritonX (Acros organics, 327371000), 1:100 dilution of proteinase K (NEB, P8107S) 2×SSC). Following overnight digestion, samples were washed for 30 min with 2×SSC and gentle agitation.

Staining with secondary probes: We used readout probes constituted by a 20 bp oligonucleotide conjugated to a fluorophore (Alexa Fluor 488, Cy3B, Cy5 or Alexa Fluor 750) via a disulfide bond. Fluorescent conjugated probes were purchased from Biosynthesis Inc. The secondary staining solution was composed of 5% ethylene carbonate (Sigma Aldrich, E26258-100G) 2×SSC. The secondary staining solution was supplemented by a secondary readout probe for each fluorescent color at a 3 nM final concentration, and with DAPI at a 1 μM final concentration. Secondary staining was conducted following the same procedure for the primary staining step with the exception that it was conducted for 20 min at room temperature, covering samples with aluminum foil. Following hybridization, samples were washed once with a 10% ethylene carbonate 2×SSC solution for 20 min with gentle agitation, and three times with 2×SSC for 5 min per wash.

Iterative smFISH imaging was performed as previously described⁵¹.

smFISH image processing and analysis: To collapse z-stacks into a single 2D image, maximum projection images were generated using the Nikon Elements software's maximum projection function. After each round of FISH imaging, we took an additional image with cleaved fluorophores to capture the background signal for each channel. As the microscope has unequal sensitivity to the 5 different fluorophores, we also imaged each fluorophore's flat field to capture its bias. We corrected raw FISH images by subtracting the background signal of each gene and then dividing by the flatfield bias of the conjugated fluorophore, then thresholding to 0 to correct any negative-valued pixels. Additional alignment was performed by using DAPI.

### Immunoblotting

Immunoblotting was performed in mESCs containing a Dox-inducible GFP-coupled shRNA^{29,32}. The genotypes used were shOgdh.2081, shOgdh.346¹ and shRen_731. mESCs were treated with doxycycline for 72 hrs. Briefly, the supernatant was removed, and the cells were washed three times with PBS. mESCs were then lysed using RIPA lysis buffer (Sigma Aldrich, R0278) supplemented with NaF (1 mM), Na₄P₂O₇ (20 mM), and Na₃VO₄ (2 mM). The lysates were incubated for 15 minutes on ice and subsequently clarified by centrifugation at 4° C. and 10,000 g. The protein concentration was determined using bovine serum albumin (BSA) (A7906, Sigma) as a standard protein. Lysates were concentrated to a final concentration of 1 mg/ml by boiling the appropriate amount of protein lysates with 2× Laemmli buffer (4% SDS, 20% glycerol, 10% 2-mercaptoethanol, 0.004% bromophenol blue in 0.2 M Tris·HCl, pH 7) at 90° C. for 10 minutes. Twenty micrograms of protein lysates were loaded onto SDS-PAGE gels and transferred to 0.2 m nitrocellulose membranes (LI-COR Bioscience, 926-31090). The membranes were blocked with 5% Blotting-Grade Blocker (non-fat dry milk, 170-6404, Bio-Rad) in Tris-buffered saline containing 1% Tween 20 for 1 hour at room temperature. Subsequently, the membranes were incubated overnight at 4° C. in TBS-T with 3% sodium azide with rabbit anti-Ogdh antibody (1:500, Proteintech, 15212-1-AP). After washing the membranes three times with TBS-T for 10 minutes each at room temperature, they were incubated with secondary anti-rabbit antibody (1:5000, Cell Signaling, 7074S) in 1% Blotting-Grade Blocker. The protein bands were visualized using enhanced chemiluminescence (ECL) detection reagent (Cell Signaling, 6883P3) according to the manufacturer's instructions.

### Chromatin Immunoprecipitation (ChIP) in Intestinal Crypts

Intestinal crypts were isolated and cross-linked for 10 minutes at room temperature (RT) in 1% formaldehyde. Cross-linking reactions were stopped by adding 1.25 M glycine to a final concentration of 125 mM. The crypts were then centrifuged for 10 minutes at 4° C. and washed in cold PBS. The cells were lysed with 1 mL of lysis buffer-1 (50 mM HEPES-KOH, pH 7.5, 140 mM NaCl, 1 mM EDTA, 10% glycerol, 0.5% NP-40, 0.25% Triton X-100, 1× protease inhibitor) followed by centrifugation at 4° C. The pellet was resuspended in lysis buffer-2 (10 mM Tris·HCl, pH 8.0, 200 mM NaCl, 1 mM EDTA, 0.5 mM EGTA, 1× protease inhibitors). Finally, the pellets were resuspended in 1 mL of lysis buffer-3 (10 mM Tris·HCl, pH 8.0, 100 mM NaCl, 1 mM EDTA, 0.5 mM N-lauroylsarcosine, 1× protease inhibitors), and 100 μL of 10% Triton X-100 was added. The samples were sonicated for 10 minutes using E220 Focused Ultrasonicator (Covaris, PN 500239). Samples were sonicated using milliTube-1 ml with AFA fiber (Covaris, PN 520130) under flowing conditions (140 pip, 5% Duty cycle, 200 CBP). The soluble fraction was quantified using the Bradford assay, and 400 g of the soluble fraction was used for immunoprecipitation of the transcription factors Hnf4α (1 mg, Thermo Scientific, MA1-199) and Smad4 (1 mg, Cell Signaling, 46535) with Rabbit IgG (1 mg, Cell Signaling, 2729S) and mouse IgG (1 mg, Santa Cruz Biotechnology, sc-2025) used as a control. The chromatin and antibody mixtures were incubated overnight at 4° C. in a total volume of 500 L. The immunoprecipitated mixture was then washed sequentially with a low salt buffer, a high salt buffer, and a LiCl wash buffer. The samples were decross-linked, and the DNA was extracted using phenol/chloroform/isoamyl alcohol mixtures, washed with 80% ethanol, and resuspended in 200 μL of TE buffer. Subsequently, qRT-PCR was performed using specific primers.

### Statistical Analysis and Data Representation

Before performing any statistical test, we tested for normal distribution using the D'Agostino & Pearson test. For continuous variables, we used student's t test, Mann-Whitney U test, one-way ANOVA or Friedman's test. For categorical variables, we used chi-squared test or Fisher's exact test. Mantel-Cox test was used to analyze Kaplan-Meier survival of mice. If significant differences by one-way ANOVA were found, group-wise comparison was done (Tukey's multiple comparison test). If significant differences by Friedman's test, were found Dunn's multiple comparisons test was used. The predictive value of Ogdh expression in enterocytes, in ISCs, and in Paneth cells was evaluated by examining the area under the receiver-operator characteristic (ROC) with a confidence interval of 95%. All statistical tests were considered statistically significant when P was below 0.05. Statistical significance in FIGURES is summarized as follows: *, P≤0.05; **, P≤0.01; ***, P≤0.001; and ****, P≤0.0001 between the means of a minimum of three samples. Results are expressed as the mean value±SEM.

IF, H & E, other stainings, and IHC are representative of at least three independent mice. Quantification of IF, H & E, and other stainings was performed for over 50 crypts or 100 villi per mouse in at least three independent mice. For in vitro experiments, at least three independent experiments were performed. For the in vivo experiments, at least five mice per group were used.

### Example 2: Metabolic State Correlates with Lineage Specification in the Intestine

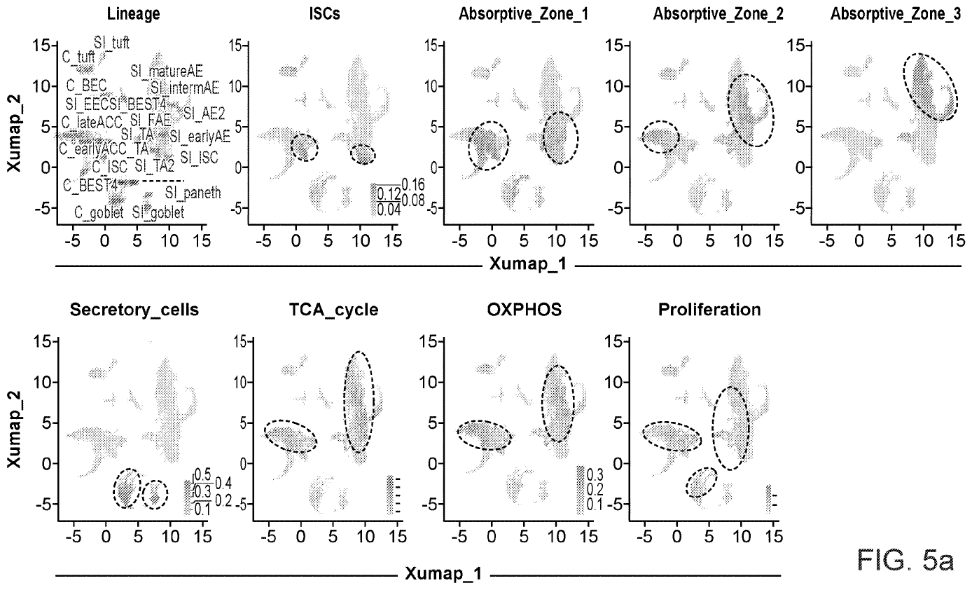

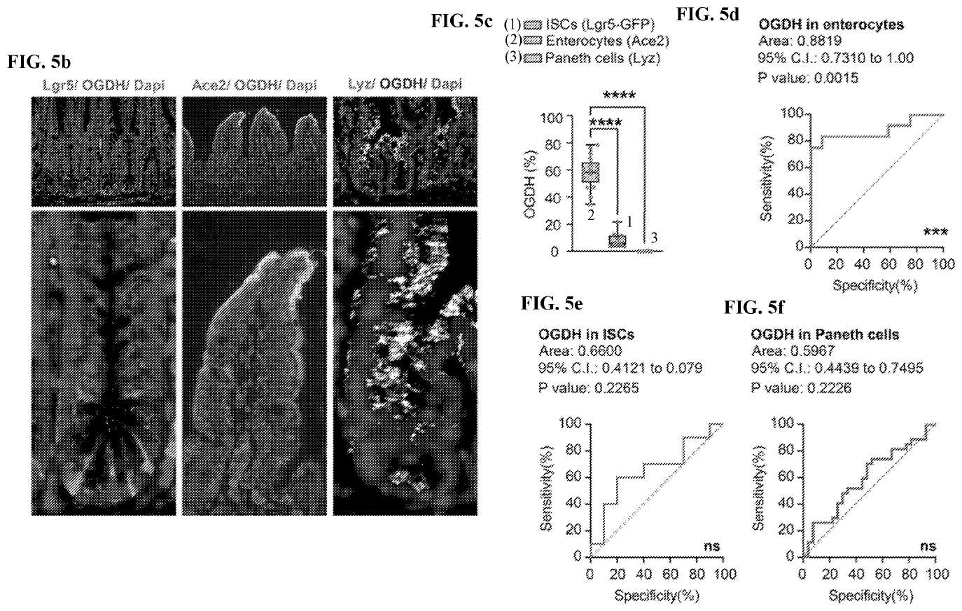

To explore the interplay between the TCA cycle and cell fate specification, we used the murine small intestine as a well-established system for studying multilineage differentiation and regeneration. Contrary to the notion of constitutive gene expression of TCA cycle enzymes, analysis of publicly available single cell RNA sequencing (scRNA-seq) of intestinal and colonic mucosa²⁴⁻²⁶ revealed unexpected heterogeneity and dynamism in the expression of the TCA cycle enzymes. Compared to the ISCs, absorptive clusters showed increased expression of nearly all the tricarboxylic acid (TCA) cycle enzymes (FIG. 1a and FIG. 5a). Conversely, the secretory lineage exhibited comparatively little induction of TCA cycle gene expression and downregulation of several enzymes (FIG. 1a and FIG. 5a). Single molecule fluorescence in situ hybridization (smFISH), immunofluorescence staining, and quantitative PCR (qPCR) analysis on sorted cells using an ISC reporter mouse (Lgr5-EGFP reporter, see Methods)²⁷ confirmed a dynamic range of enzyme expression in the different intestinal lineages and revealed an significant difference in the TCA cycle beginning with the level of Ogdh (FIGS. 1b-1g and FIGS. 5b-5f). Specifically, Ogdh expression was elevated in the absorptive lineage but downregulated in secretory cells when compared to ISCs (FIGS. 1b-1g and FIGS. 5b-5f). This heterogeneity in TCA cycle enzyme expression across lineages suggests a markedly different metabolism among progenitors and further suggests that ISCs could modulate metabolite abundance to exert precise control over gene expression and lineage trajectory.

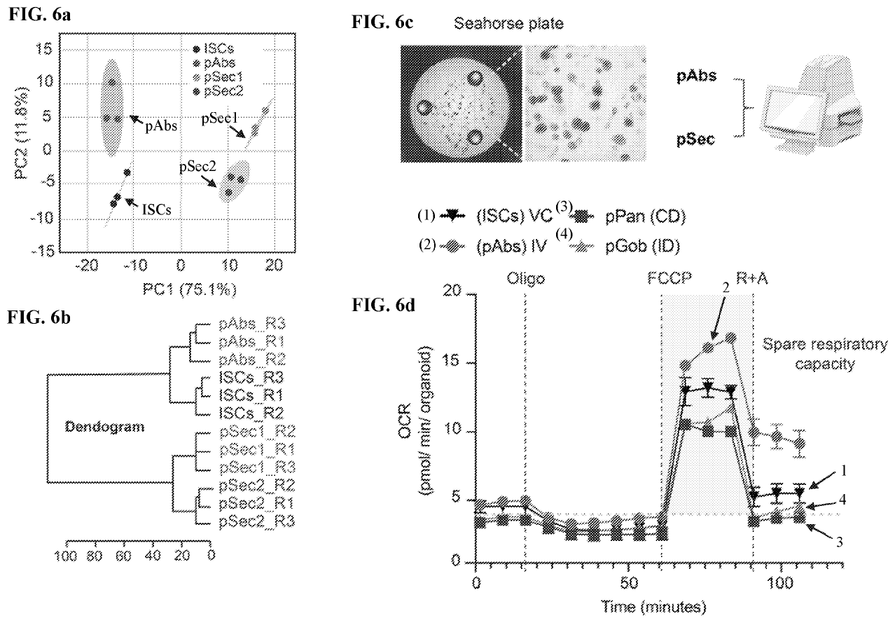

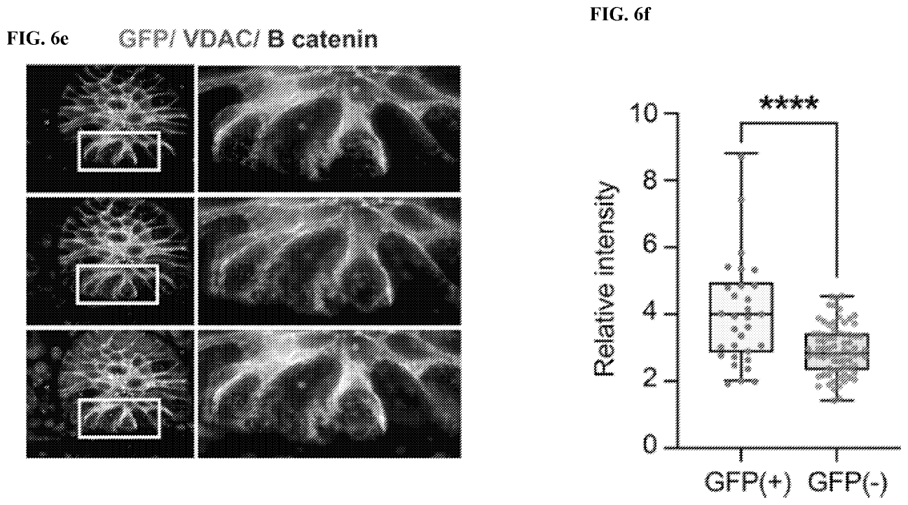

To gain insights into the metabolic effects associated with the marked variation in TCA enzyme gene expression, we used an organoid model that directs differentiation into distinct intestinal lineages through specific culture conditions²⁸. After confirming that these systems recapitulate the cell type fluctuations in Ogdh observed in different intestinal lineages in vivo (FIG. 1h-1j), we examined metabolite profiles in ISC-enriched, enterocyte-enriched (pAbs), Goblet-enriched (pSec1), and Paneth-enriched (pSec2) organoids using in ion-pair liquid chromatography coupled with tandem mass spectrometry (LC-MS/MS). These analyses identified 299 metabolites across conditions that could clearly distinguish absorptive and secretory progenitors from ISC-enriched organoids (FIG. 1k and FIGS. 6a, 6b). The pAbs displayed a relative increase of metabolites implicated in biosynthetic (dUTP, dXMP, GDP, GTP) and bioenergetic processes (ATP). In contrast, pSec1 and pSec2 exhibited elevated levels of citrate, isocitrate, and alpha-ketoglutarate (αKG), but reduced levels of subsequent TCA cycle intermediates. As a result, pSec1 and pSec2 increased their αKG/succinate ratio while pAbs did not (FIGS. 1l, 1m). These metabolic differences were consistent with the lower expression of Ogdh in the secretory versus absorptive lineages (FIG. 1m).

The observed changes in TCA gene expression and metabolite abundance suggest that the intestinal lineages may have functionally different TCA cycle capabilities and mitochondria abundance. The TCA cycle serves as a source of reducing equivalents that fuel the electron transport chain and are ultimately used to reduce oxygen. Accordingly, oxygen consumption during mitochondrial functional assays demonstrated that secretory progenitors had less spare respiratory capacity in comparison to ISCs and absorptive progenitors (FIGS. 6c, 6d). Furthermore, immunofluorescence staining using a reporter mouse to pinpoint ISCs (Lgr5-EGFP-CreERT2)²⁷ showed that, compared to ISCs, secretory Paneth cells had a decrease in mitochondria (FIGS. 6e-6f). Together, these findings suggest that distinct metabolic transitions accompany ISC differentiation into the secretory and absorptive lineages, involving an upregulation of mitochondrial dependency in the absorptive lineage and reduced level of use of mitochondrial OXPHOS capacity in the secretory lineage. They also suggest that reduced Ogdh expression facilitates an increase the αKG/succinate ratio during secretory lineage specification.

### Example 3: Dual Role of Ogdh in Lineage Specification During Tissue Regeneration

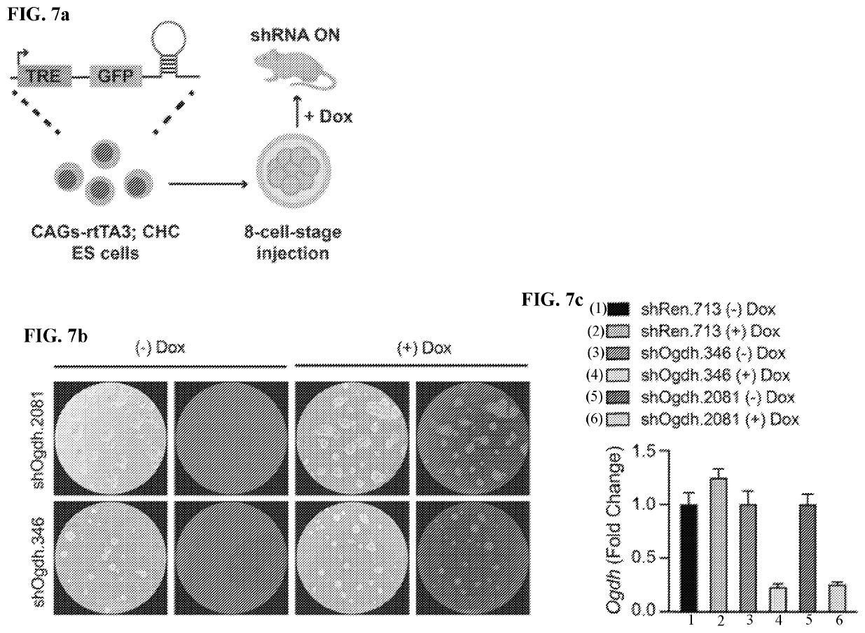

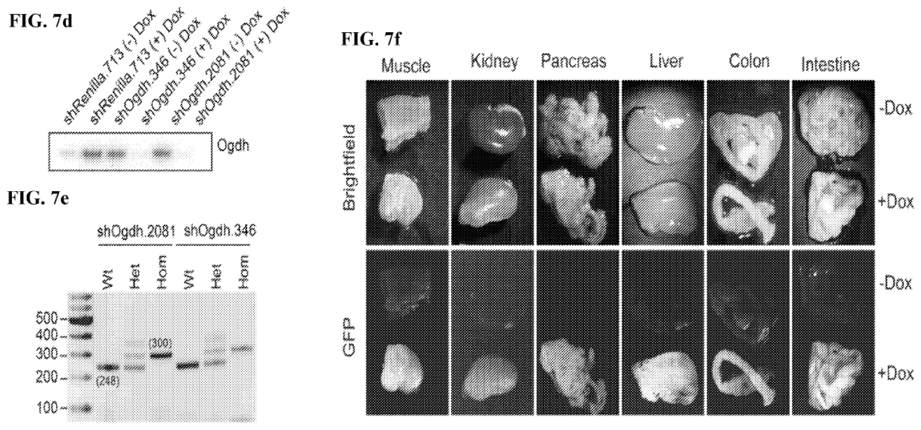

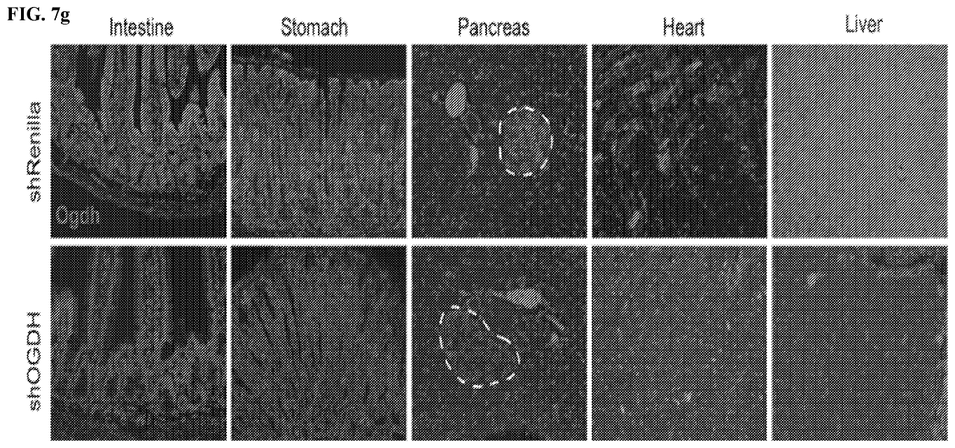

To investigate the role of OGDH during intestinal regeneration, we developed a transgenic mouse model permitting doxycycline-inducible shRNA mediated silencing²⁹⁻³¹ of Ogdh in vitro and in vivo and in intestinal organoid culture (TRE-shOgdh, see Methods). Inducible and potent GFP induction and Ogdh suppression were confirmed in ES cells prior to mouse production (FIGS. 7a-7d), and two distinct Ogdh shRNAs (shOgdh.2081 and shOgdh.346) were used to minimize the potential for off-target effects. Following the generation of germline strains, we crossed TRE-shOgdh mice with a CAGs-rtTA3 transgenic strain to enable widespread inducible expression of the TRE-GFP-shRNA cassette, including in the intestine³² (FIGS. 7e-7g). ISC-enriched organoids were derived from these TRE-shOgdh^{Cag-rtTA3} mice and from control TRE-shRen^{Cag-rtTA3} mice (harboring a neutral Renilla luciferase-targeting shRNA). Organoids were maintained in ISC-enriching or differentiation conditions, treated with doxycycline, and analyzed for lineage specification, proliferation, and cell death. Of note, in all the studies to follow, we noted similar phenotypes with both Ogdh targeting shRNAs. Because OGDH suppression is predicted to both perturb TCA function and increase αKG levels, we performed parallel studies in wild-type organoids treated with a cell-permeable dimethyl (DM)-αKG to help disentangle these effects.

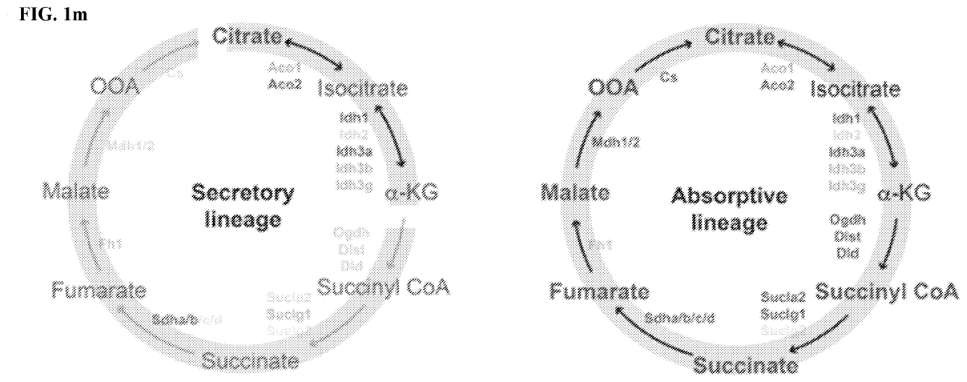

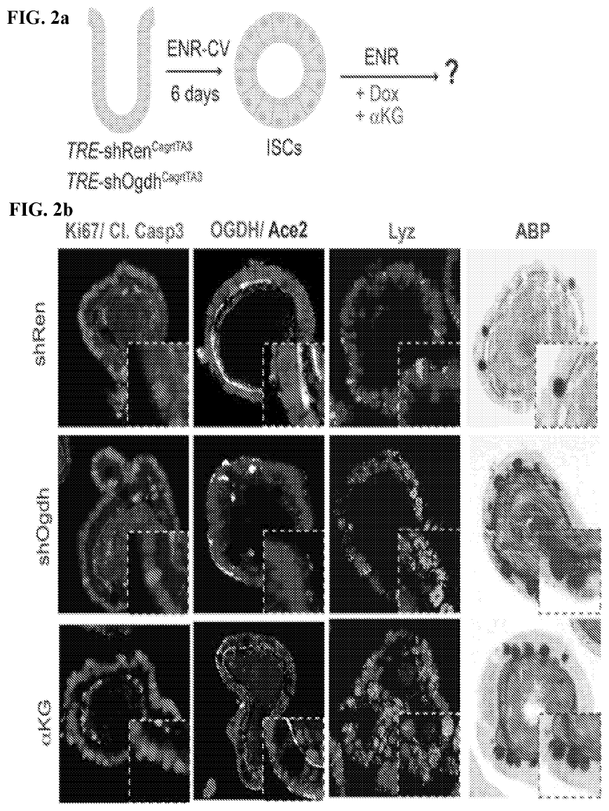

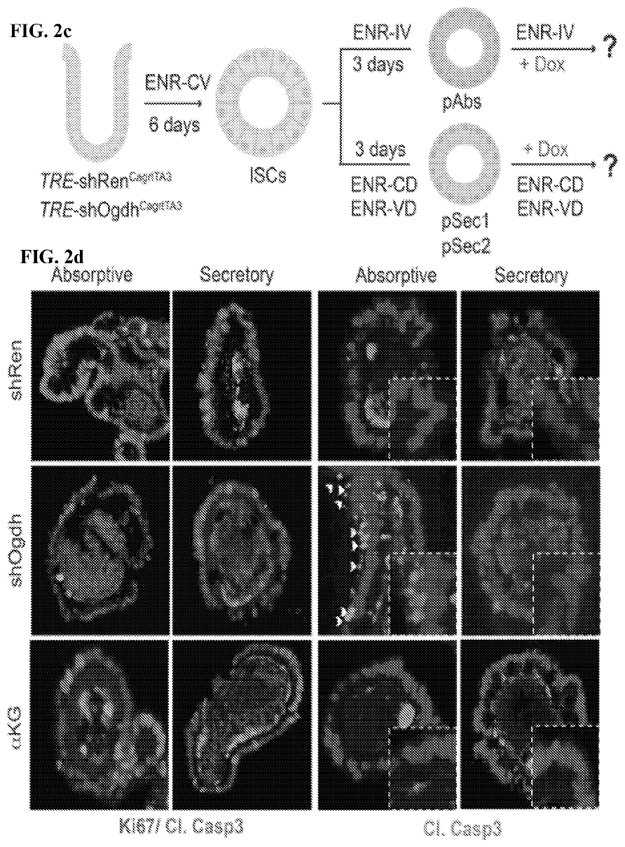

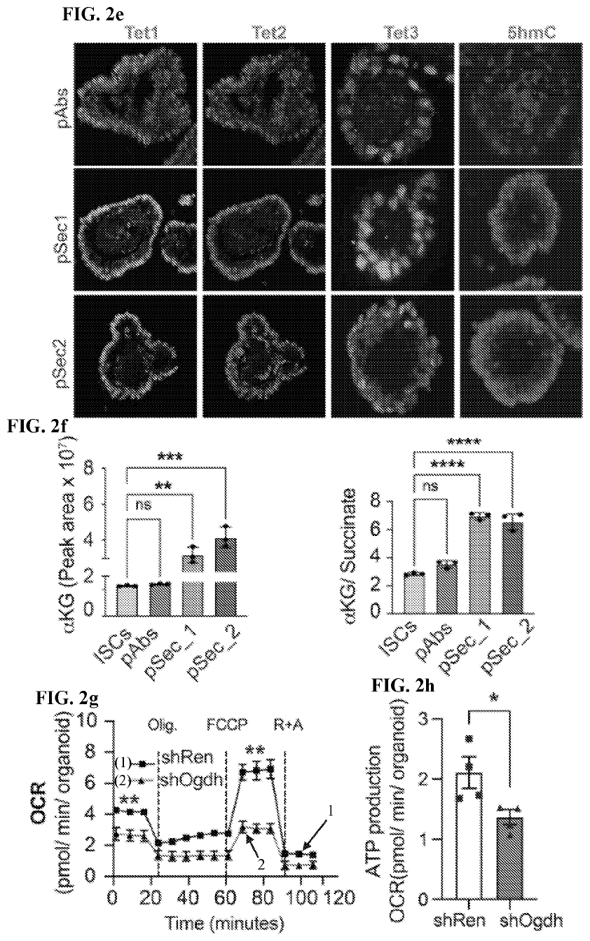

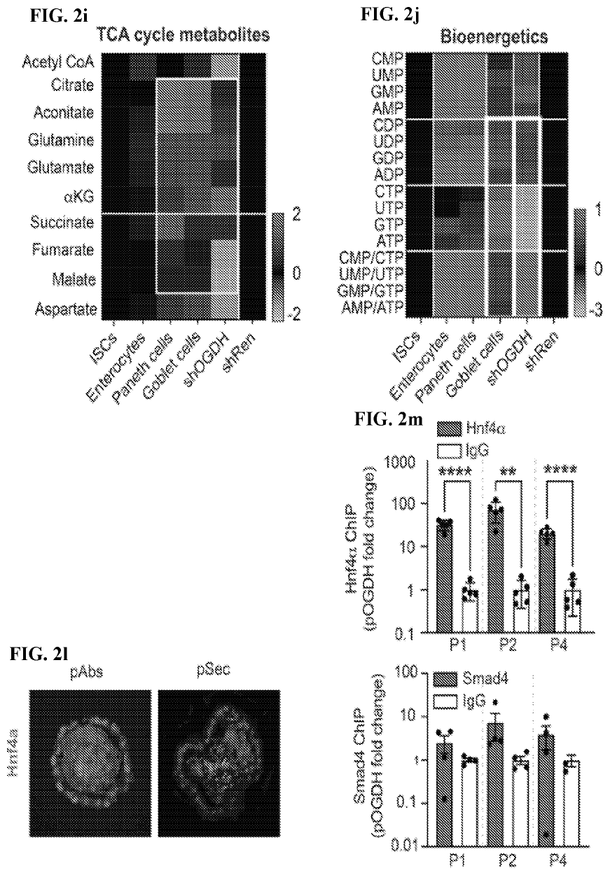

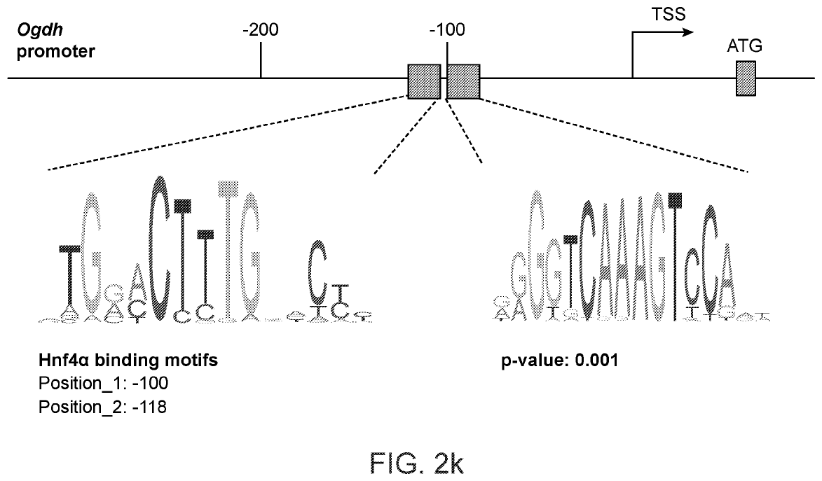

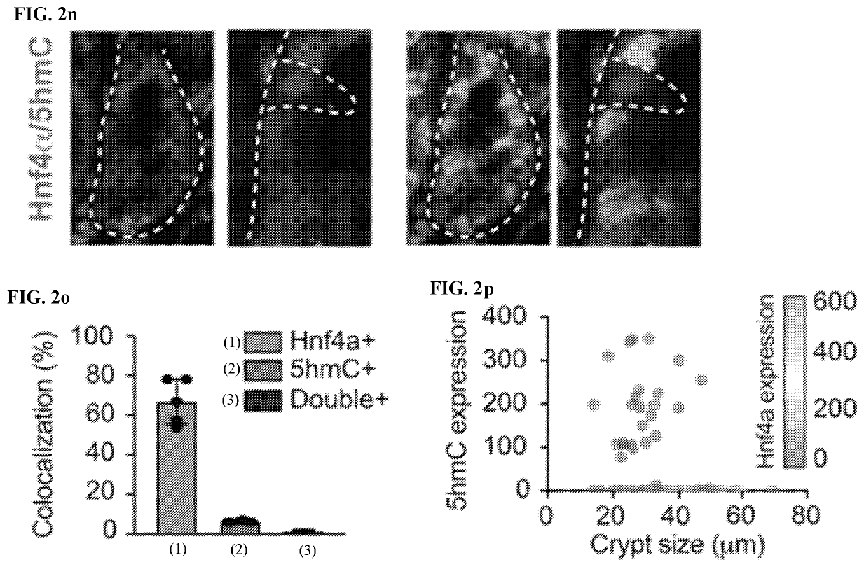

The impact of Ogdh knockdown and exogenous αKG addition resulted in remarkably lineage-specific variations in differentiation, proliferation, and cell viability. In ISC-enriched organoids, Ogdh knockdown biased differentiation towards the secretory lineage, as evidenced by increased Alcian Blue-PAS (ABP) staining for goblet cells and increased lysozyme expression for Paneth cells. Strikingly, DM-αKG achieved a similar effect (FIGS. 2a-2b). No cell death was observed following either perturbation, which is in line with previous reports that ISCs depend primarily on glycolysis for ATP generation and that a reduction in mitochondrial capacity is required for secretory lineage establishment³³. In pSec1- and pSec2-enriched organoids, neither Ogdh knockdown nor αKG supplementation altered differentiation, proliferation, or death (FIGS. 2c-2d), results consistent with the already low levels of Ogdh expression and reduced reliance on mitochondrial OXPHOS for energy production in the secretory lineage³³. In stark contrast, Ogdh knockdown, but not αKG supplementation, triggered a marked reduction in proliferation of pAbs populations within 3 days and substantial cell death by day 8 (FIG. 2c-2d). Collectively, these findings support a model in which low Ogdh levels facilitate an increase in αKG available to drive ISC differentiation towards the secretory lineage and reinforce the notion that Ogdh upregulation (alongside increases in other TCA enzymes expression) is critical in supporting the bioenergetic and/or biosynthetic demands required for enterocytic lineage specification.

### Example 4: Dissection of Lineage-Specific Effects of TCA Cycle Regulation in the Absorptive and Secretory Lineages

Next, we compared the factors underlying the differential effects of Ogdh knockdown in the distinct intestinal lineages. The perturbation experiments above indicate that increased levels of αKG influence ISC differentiation towards the secretory lineage. One possible mechanism is activation of αKG-dependent dioxygenases, such as ten-eleven translocated (Tet) enzymes. Although Tet enzyme expression was similar between absorptive and secretory lineages, levels of 5hmC, an output of Tet activity^{2,23}, were higher in secretory cells than in absorptive cells (FIGS. 2e, 2f). These results suggest that, under physiological conditions, the secretory lineage decreases Ogdh levels to achieve an activating threshold of αKG, which leads to increased activity in αKG-dependent dioxygenases.

By contrast, the proliferative arrest and cell death observed in pAbs following Ogdh knockdown was consistent with a catastrophic metabolic decline in this lineage. Consistent with an impairment in mitochondrial respiration capacity and a possible bioenergetic failure induced by energy stress in the enterocyte lineage, Ogdh knockdown impaired basal respiration, spare respiratory capacity, and ATP generation in mitochondrial functional assays (FIGS. 2g, 2h). Analysis of metabolite levels revealed that Ogdh knockdown produced a broad reduction in TCA intermediates, most notably in fumarate and malate, and TCA cycle derivatives such aspartate (FIG. 2i), the need for which contributes to OGDH dependence of certain tumor cells³⁴. As reported above, steady-state levels of these metabolites are present at much lower in pSec1 and pSec2 progenitors, and Ogdh knockdown did not further compromise cells committed to the secretory lineage (FIG. 2i). In contrast, Ogdh-depleted organoids showed an increase in their AMP:ATP ratio as well as other monophosphate:triphosphate ratios (FIG. 2j), a condition that can trigger cell cycle arrest and is a harbinger of cell death³⁵⁻³⁷. Thus, the depletion of Ogdh in absorptive progenitors poses a dual, but lineage-specific challenge in terms of bioenergetics and biosynthesis, as absorptive cells distinctly depend on TCA cycle reactions for metabolic homeostasis compared to secretory cells.

While the control of TCA cycle output between the absorptive and secretory lineages is likely multi-factorial, we noted the OGDH promoter has binding sites for the HNF4α transcription factor, a master regulator of the enterocyte lineage³¹ (FIG. 2k). Consistent with the key role that Hnf4α plays in absorptive lineage differentiation³⁸, Hfn4α protein levels were substantially higher in pAbs compared to pSec1 and pSec2 progenitors (FIG. 2l). Chromatin-immunoprecipitation assays also revealed specific binding of HNF4α to the Ogdh promoter in the murine intestine (FIG. 2m). These observations suggest a role for HNF4α in driving the Ogdh levels needed for the bioenergetic and biosynthetic demand of this lineage. By contrast, reductions in Ogdh levels during differentiation of the secretory lineage apparently enable chromatin-related changes that facilitate this cell fate transition. Accordingly, we found that HNF4α-positive cells and 5 hMC-high cells were mutually exclusive in the murine intestine (FIGS. 2n-2p).

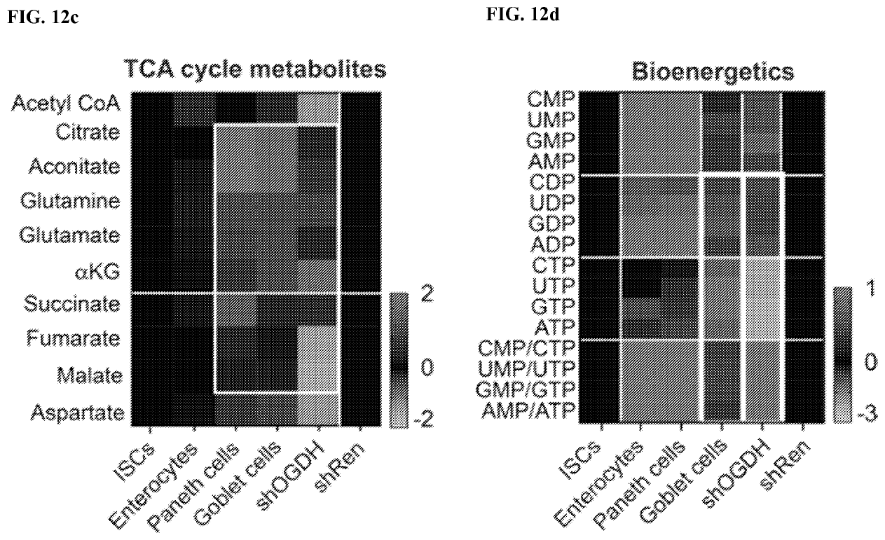

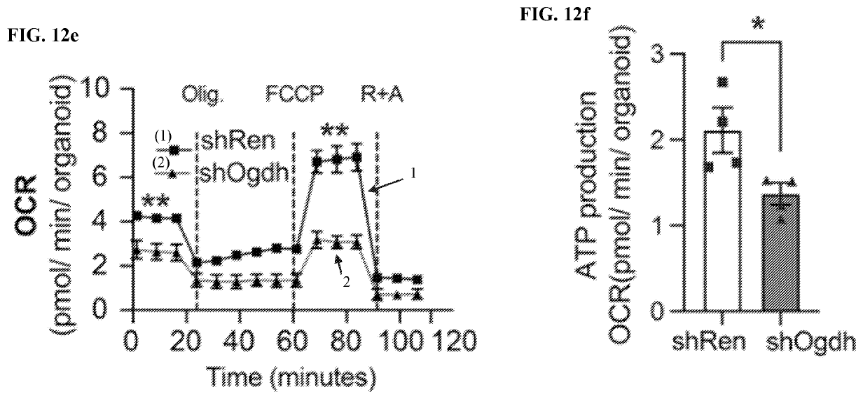

There is significant variability in Ogdh dependency among intestinal populations. In cell populations characterized by high bioenergetic and biosynthetic demand, Ogdh depletion triggers bioenergetic and biosynthetic deficits, leading to cell cycle arrest and cell death. Conversely, less bioenergetically demanding populations, such as secretory cells, exhibit no noticeable effects from Ogdh depletion. Importantly, these effects manifest exclusively when stem cells have already committed to a specific lineage, and they enter progenitor state (FIG. 12).

### Example 5: Role of Ogdh During Intestinal Homeostasis In Vivo

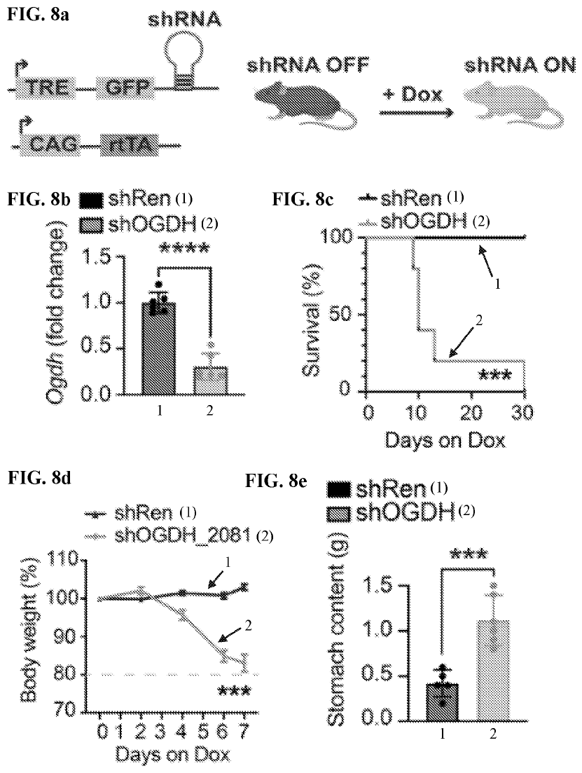

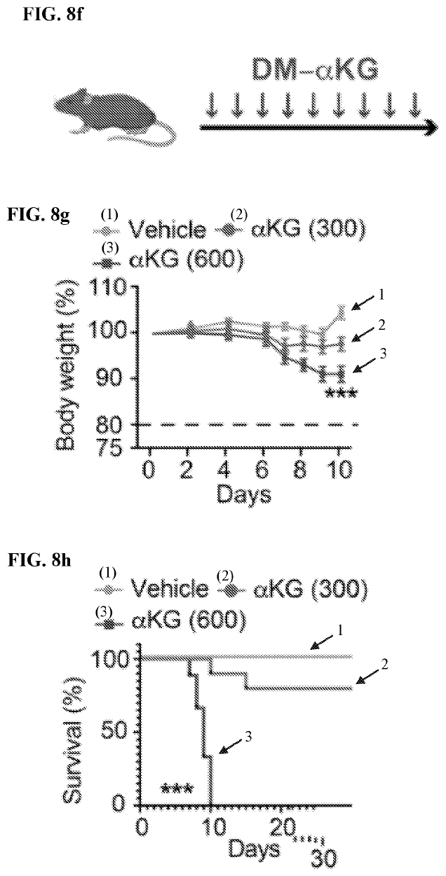

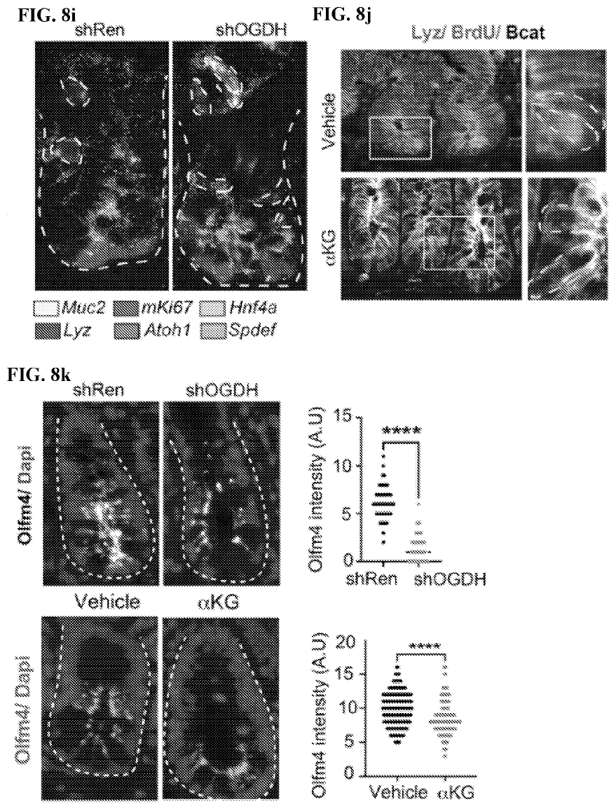

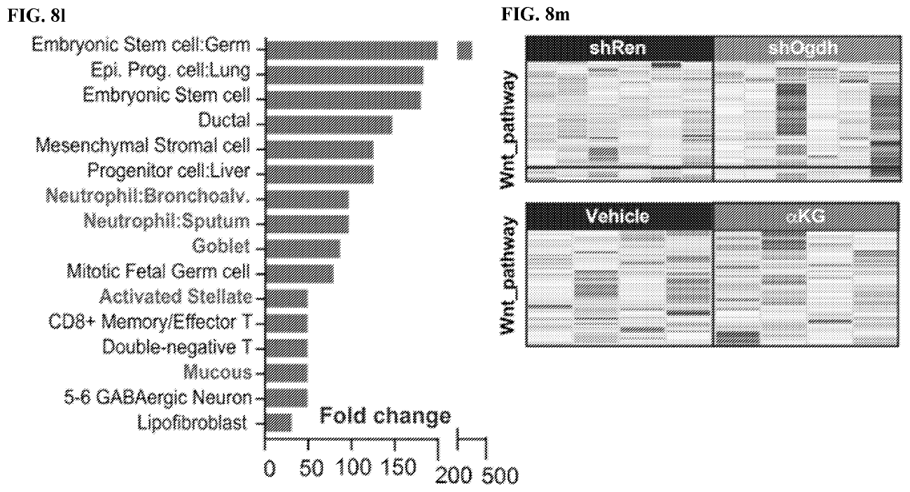

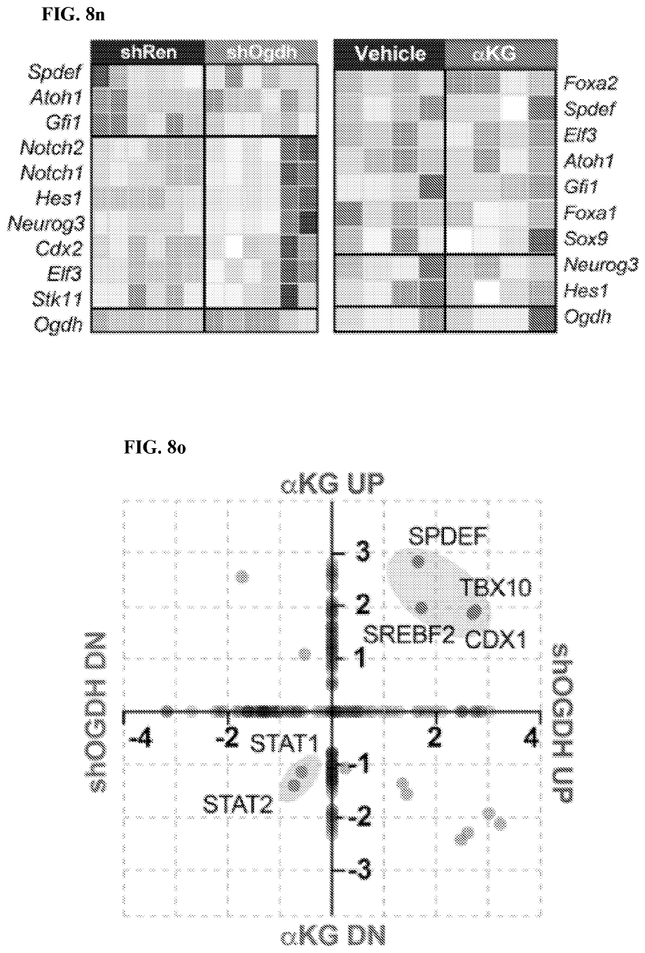

To determine whether Ogdh activity influences intestinal homeostasis in vivo, we assessed the phenotypic impact of Ogdh knockdown on the intestine in TRE-shOgdh^{Cag-rtTA3} transgenic mice (FIGS. 8a, 8b). Once again, to distinguish between the phenotypes resulting from direct effects of Ogdh suppression on the TCA cycle and those associated with increased αKG levels, we compared the effects of Ogdh knockdown with intraperitoneal administration of DM-αKG³.

Compared to TRE-shRen^{Cag-rtTA3} control, most TRE-shOgdh^{Cag-rtTA3} mice experienced a profound loss of body weight and had to be sacrificed within 7 to 11 days after the initiation of doxycycline treatment (FIGS. 8c, 8d). The Ogdh-depleted mice had dilated stomachs containing accumulated food (FIG. 8e). Furthermore, the small intestines appeared swollen but lacked luminal content, suggesting bowel obstruction leading to severe digestive defects. Although high doses of DM-αKG (600 mg/kg) were toxic, causing body weight loss, the specific pathologies (including mesenteric inflammation; not shown) were quite distinct from those observed with OGDH depletion. Moreover, mice treated with lower doses (300 mg/kg dose) were viable and did not show significant weight loss or impaired intestinal structure, which appeared normal by gross analysis (FIGS. 8f-8h).

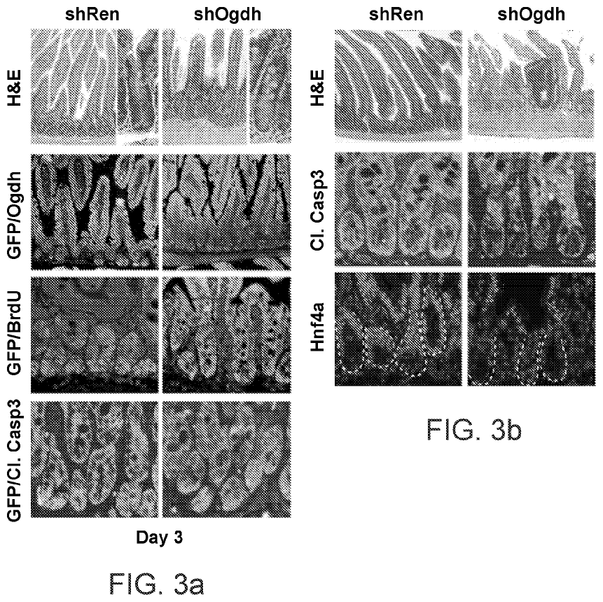

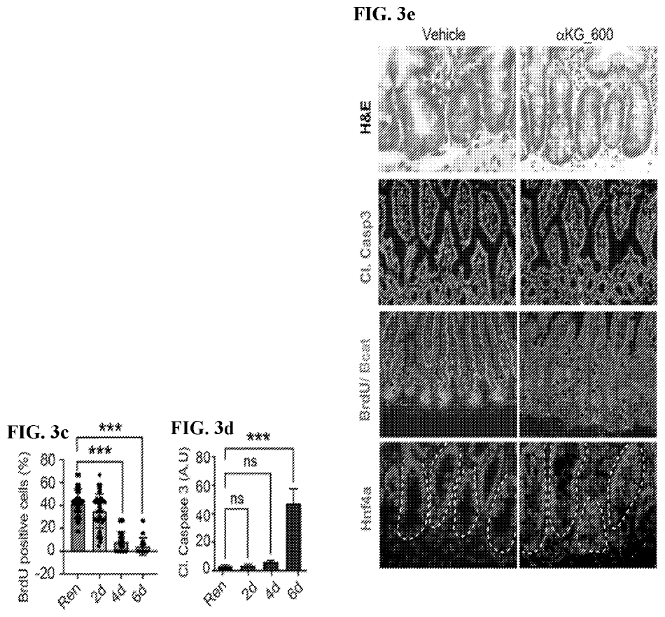

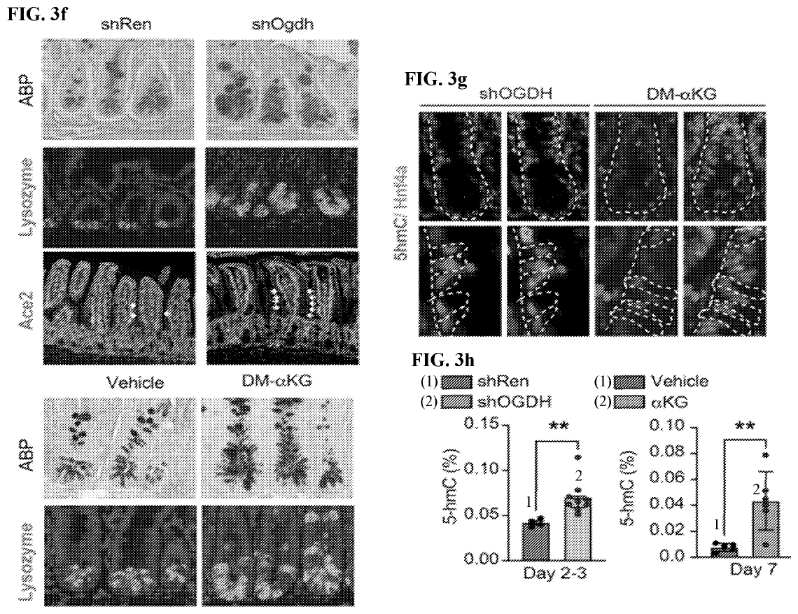

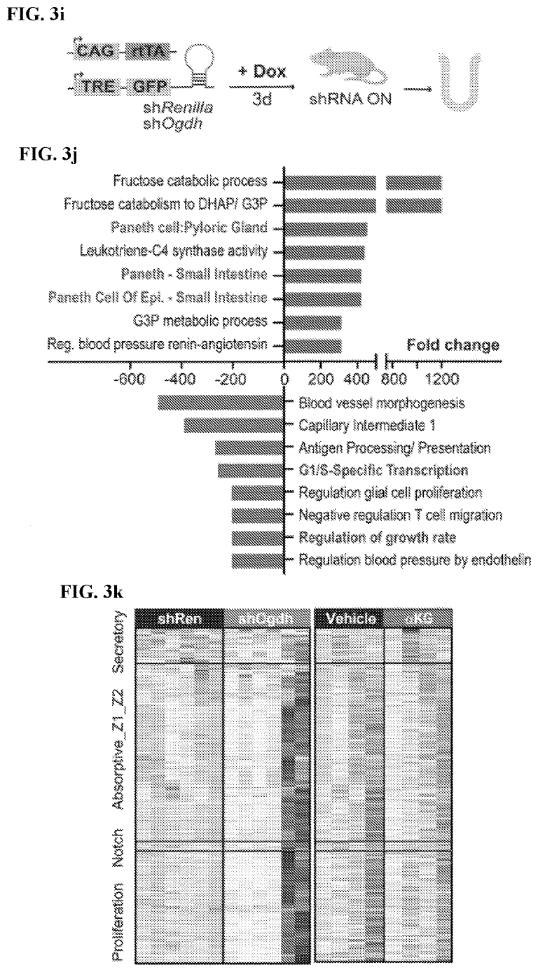

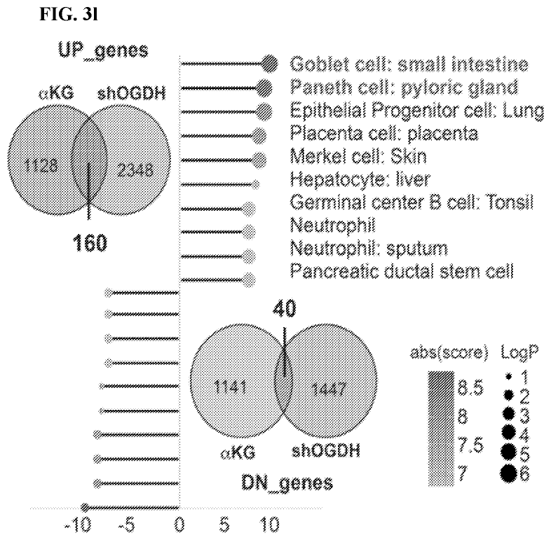

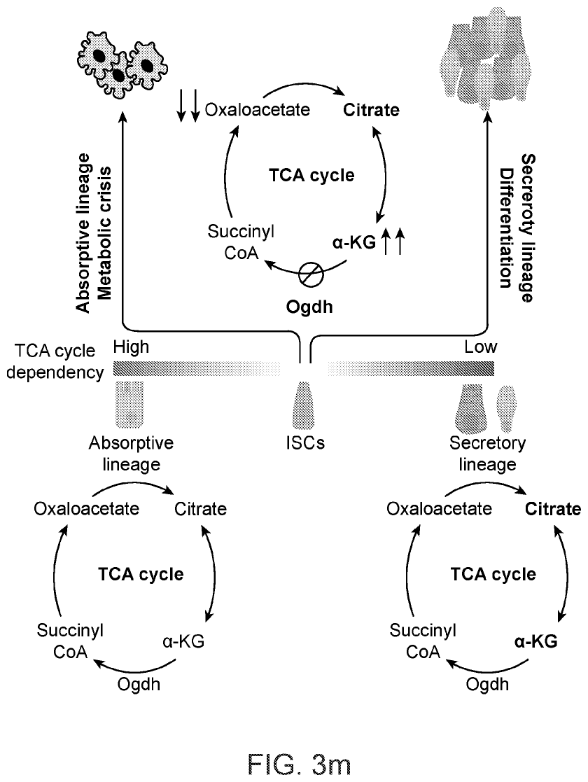

Histological and immunofluorescence analysis decisively showed OGDH knockdown and αKG supplementation had distinct effects on intestinal physiology. OGHD knockdown produced a substantial decrease in proliferation as early as 3 days after initiation of doxycycline (FIGS. 3a-3d). By day 6, this proliferative arrest was accompanied by notably increased apoptosis, primarily in the upper region of the crypts, leading to crypt hypoplasia and reductions in Hnf4α-positive cells (enterocytic progenitors) (FIGS. 3a-3d), highlighting that OGDH expression is necessary for proper intestinal homeostasis. In stark contrast, DM-αKG treatment, even at the higher dosage, did not induce either cell cycle arrest or cell death in the crypt compartment (FIG. 3e). Despite these contrasting effects in enterocytes, both Ogdh knockdown and αKG supplementation increased the abundance of newly formed and mature secretory cells (FIG. 3f and FIGS. 8i, 8j). In the Ogdh-knockdown mice, these effects preceded the onset of cell death in the crypt. The number of Olfm4+ cells (ISCs) was also reduced, supporting an augmented differentiation of the ISCs into secretory cells in vivo (FIG. 8k). Consistent with elevated Tet enzyme activity, Ogdh knockdown and αKG supplementation led to increases in the numbers of 5hmC positive cells (FIGS. 3g, 3h).

To gain granularity on the changes accompanying Ogdh knockdown and αKG supplementation in vivo, we performed RNA-sequencing on isolated intestinal crypts. Analysis of these data confirmed that Ogdh knockdown mice had a substantial reduction in the transcriptional programs associated with proliferation and the absorptive lineage specification, while DM-αKG treatment did not show this pattern (FIGS. 3i-3k and FIGS. 8l-8n). In contrast, transcriptional profiles associated with secretory cell lineage specification were markedly enriched in both TRE-shOgdh^{Cag-rtTA3} and DM-αKG-treated mice (FIGS. 3j-3l and FIGS. 8l-8n). With both perturbations, one of the top upregulated transcription factors was Spdef (Paneth cell- and goblet cell-specific transcription factor) (FIG. 8o).

These in vivo findings indicate that increases in αKG levels can induce differentiation into the secretory lineage, yet non-αKG-mediated functions of OGDH are necessary for the expansion of the enterocytic lineage (FIG. 3m). In addition, downregulation of Ogdh levels specifically in the secretory lineage apparently increases aKG levels to an extent that can activate αKG-dependent dioxygenases and promoting transcriptional programs associated with that lineage.

### Example 6: Metabolic Interventions to Treat Ulcerative Colitis

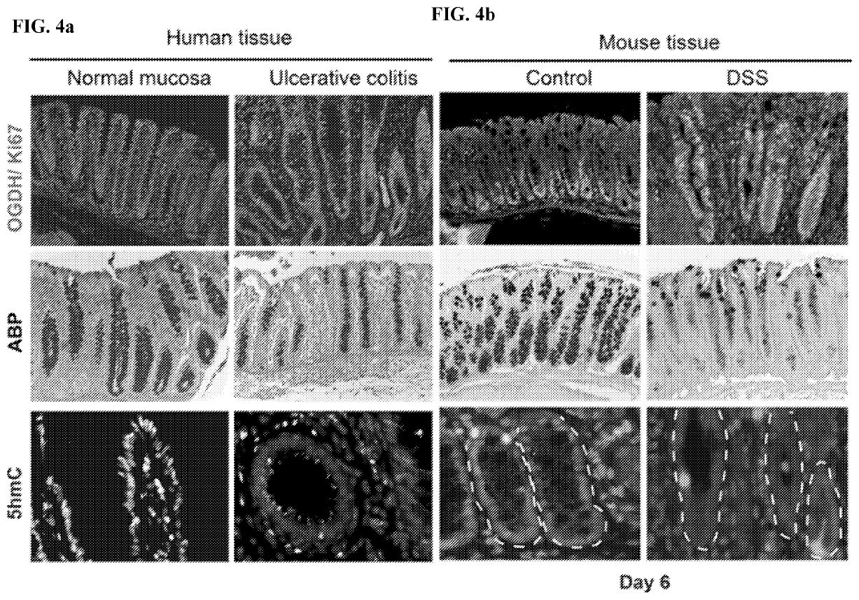

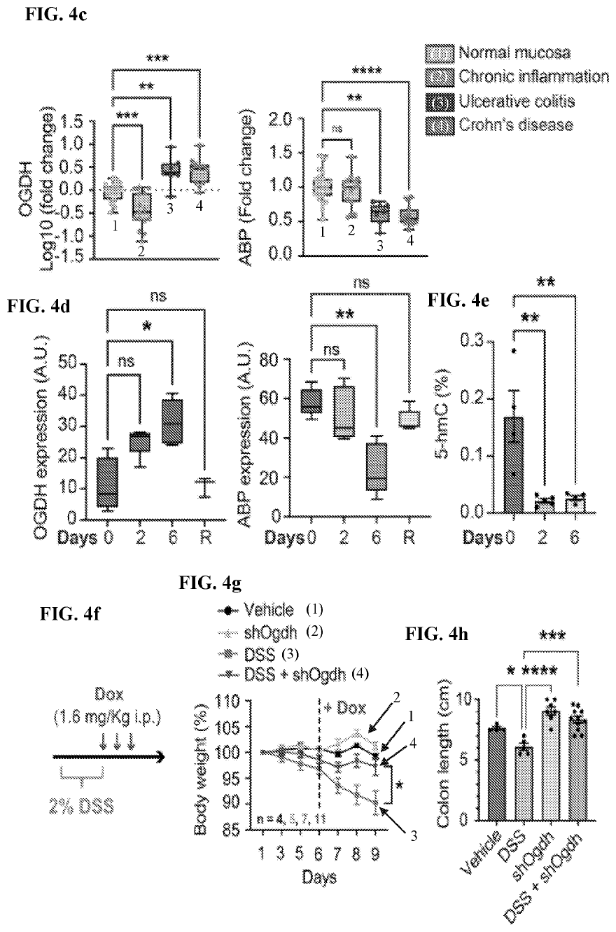

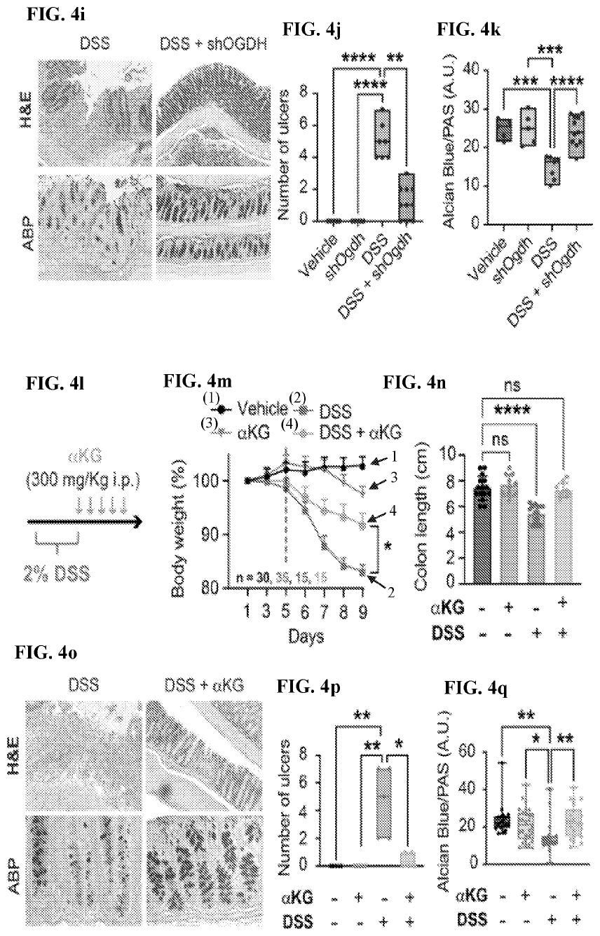

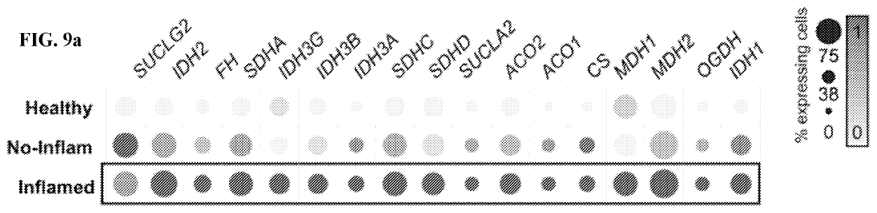

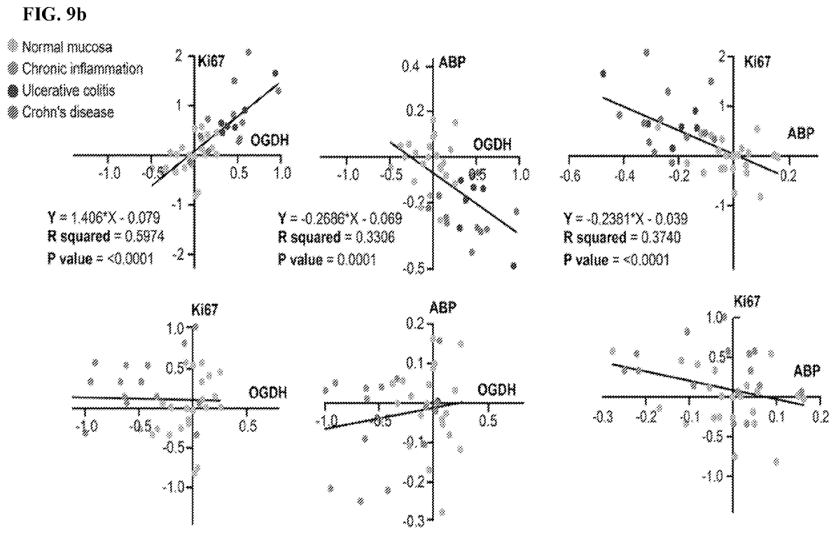

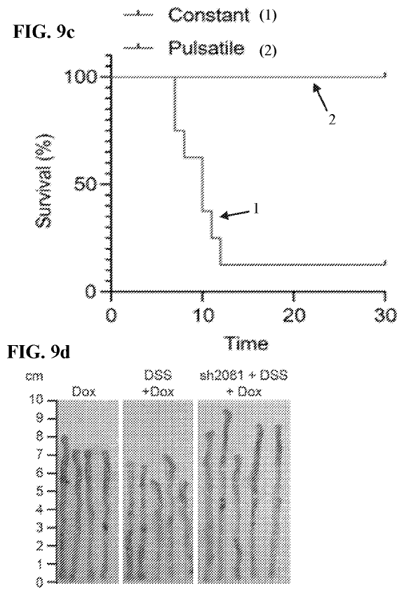

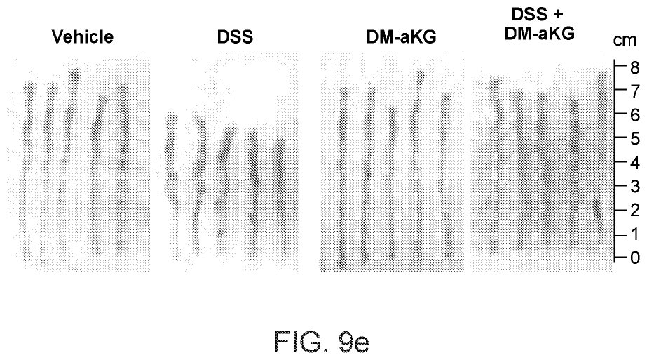

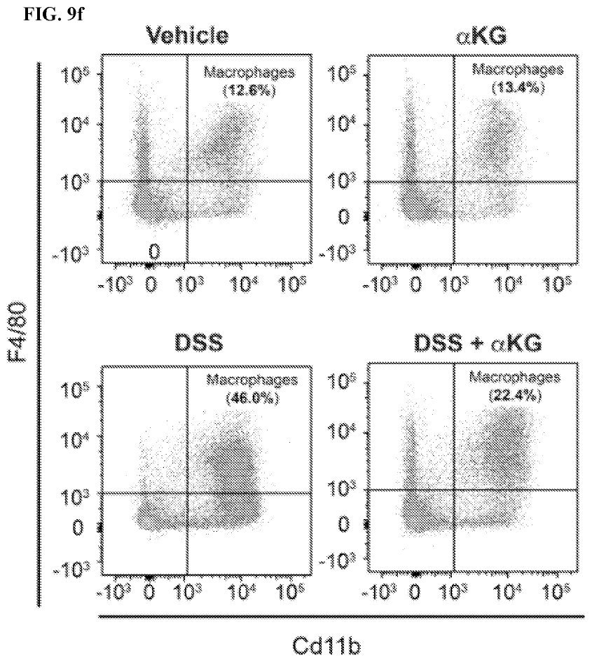

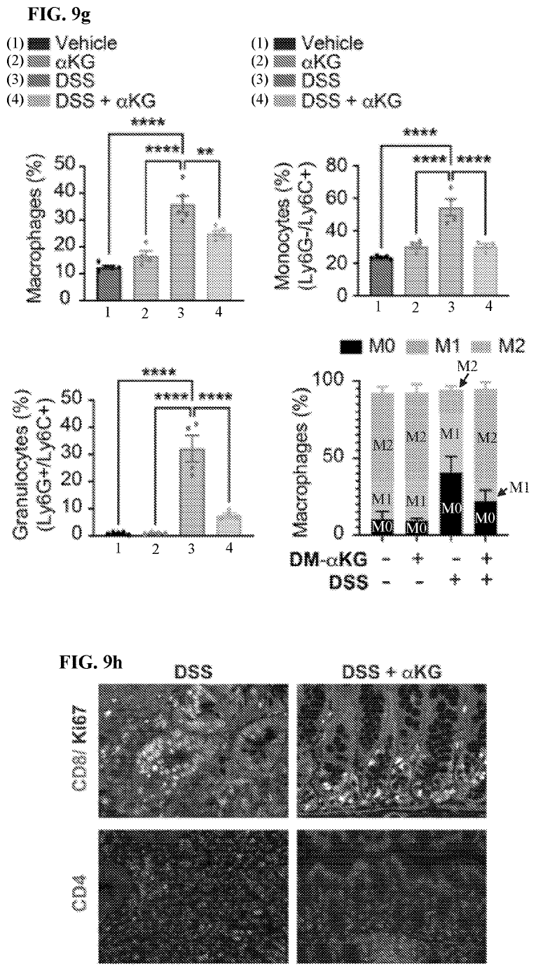

Alteration of intestinal differentiation is relevant to a series of enteropathies, including inflammatory bowel disease (IBD). IBD encompasses two major pathologies: ulcerative colitis (UC) and Crohn's disease, which are characterized by chronic inflammation in the digestive tract and impaired differentiation of the secretory lineages⁴⁻⁷. Intriguingly, analysis of scRNA-seq data from human IBD³⁹ and immunofluorescence staining on tissue microarrays from individuals with normal mucosa, chronic inflammation, ulcerative colitis, or Crohn's disease revealed a significant increase in the number of OGDH-positive cells in the IBD samples (FIGS. 4a, 4c and FIGS. 9a, 9b). This elevation correlated with higher levels of proliferation, decreased numbers of secretory cells, and decreased levels of staining (FIGS. 4a, 4c and FIGS. 8a, 8b). Similar features were observed in C57Bl/6 mice treated with dextran sulfate sodium (DSS), a chemical that directly targets the colonic epithelium and causes injury to epithelial cells (FIGS. 4b-4e). Thus, the DSS model recapitulates human IBD by mimicking effects on lineage specification and OGDH expression. In both the DSS model and patient tissues, IBD intestinal tissue displays impaired formation of the secretory lineages.

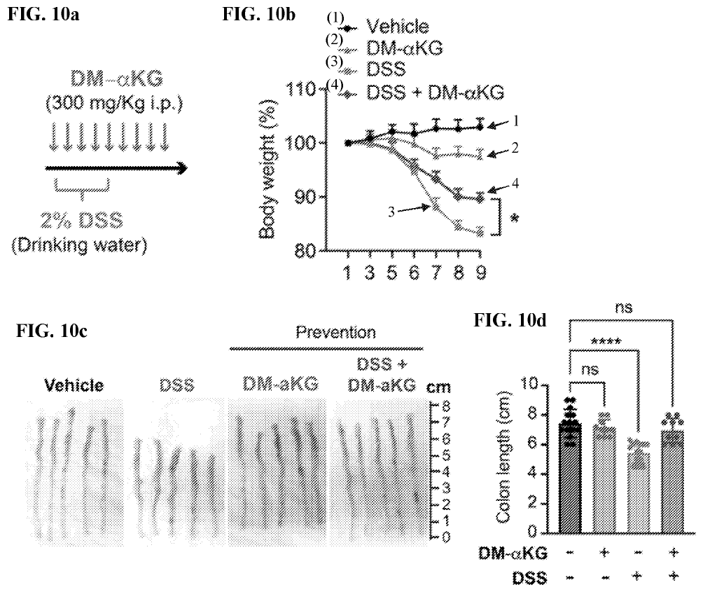

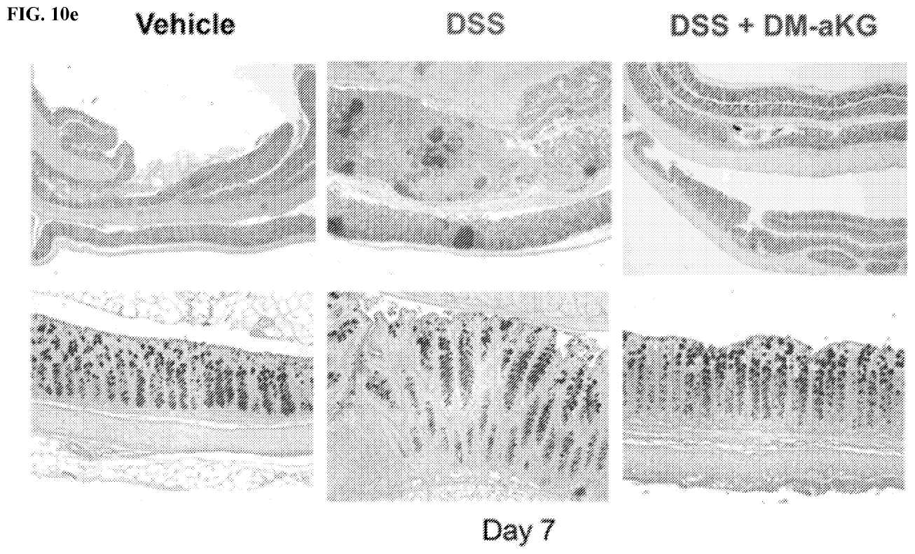

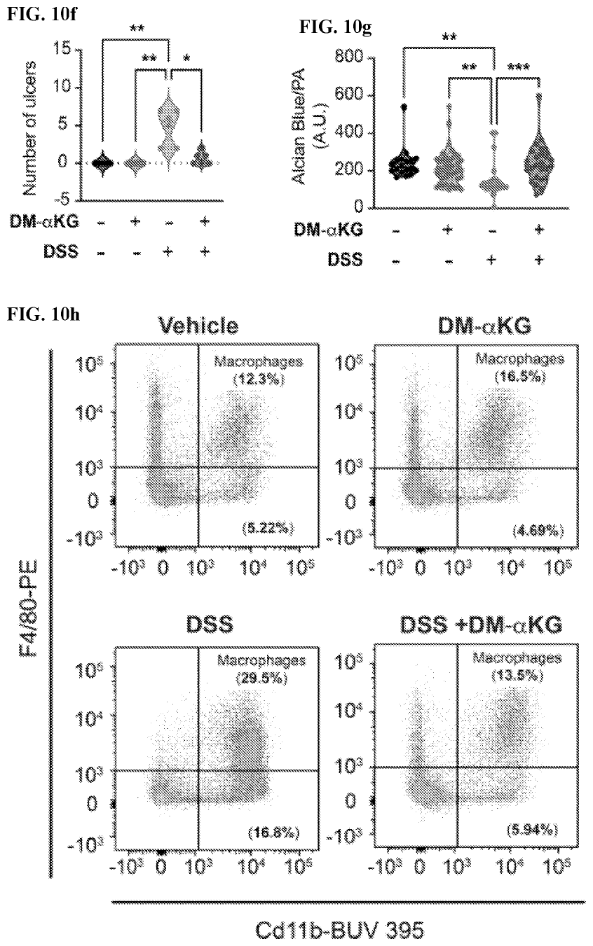

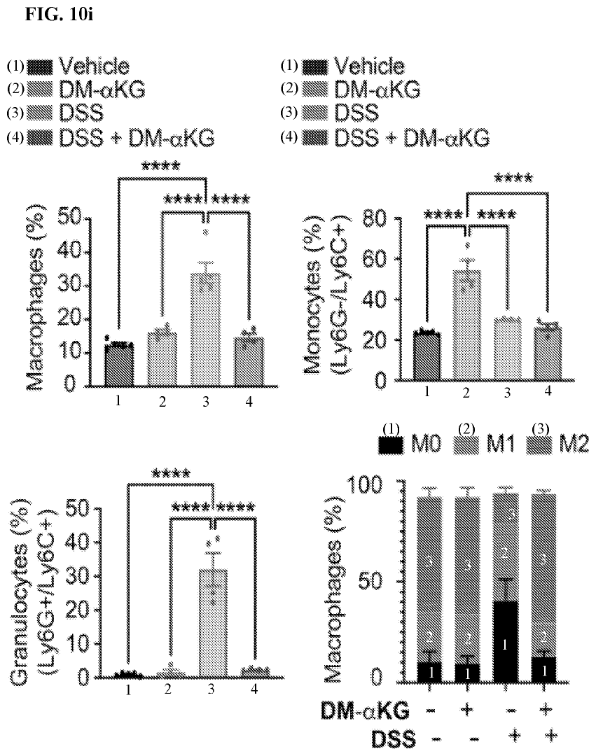

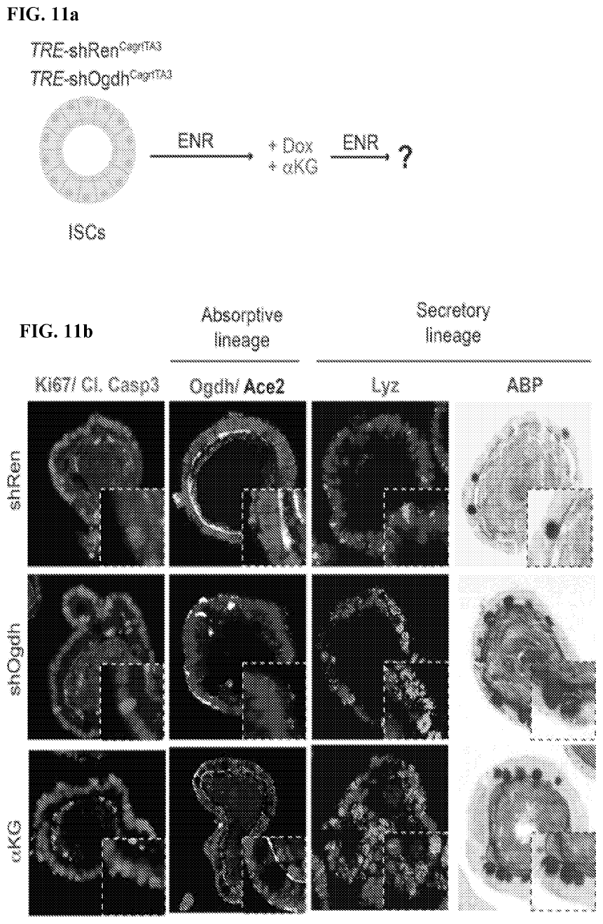

We hypothesized that perturbations that increase αKG levels could drive secretory differentiation in the DSS model, leading to an improvement in colitis-associated pathologies. To test this idea, we first induced colitis in TRE-shOgdh^{Cag-rtTA3} mice by treating with DSS for 5 days. Subsequently, the mice received pulsatile inhibition of OGDH through doxycycline injections (cycles of 3 consecutive days on followed by 4 days off) (FIG. 4f). Pulsatile depletion was chosen because, in contrast to constitutive Ogdh depletion, it produced no obvious decline in animal well-being (FIG. 9c). Ogdh suppression resulted in decreased body weight loss in response to DSS-induced colitis, as well as significant recovery of the colonic mucosa and reduced number of ulcers at 9 days after DSS treatment (FIGS. 4g-4j and FIG. 9d). Furthermore, the number of mature secretory cells in these mice increased (FIGS. 4g-4k). Similar results were observed when colitis was induced by DSS in C57Bl/6 mice and followed by daily intraperitoneal administration of 300 mg/kg of DM-αKG. Protection was observed both when DM-αKG treatment began concurrently with colitis induction by DSS, mimicking a preventative context, or 5 days after administration of DSS, mimicking an intervention context (FIGS. 4l-4q and FIG. 9e and FIG. 10). Additionally, the administration of DM-αKG in both regimens resulted in remodeling of immune cell infiltration, showing reduced levels of macrophages, monocytes, granulocytes, and infiltrating T cells, in DM-αKG-treated mice when compared to DSS alone (FIGS. 9f-9h and FIG. 10).

It is anticipated that DLD/DLST inhibitors will also induce differentiation into the secretory lineage and ameliorate symptoms of DSS-induced colitis (e.g., significant recovery of the colonic mucosa and reduced number of ulcers). Altogether, these findings underscore the potential of metabolic interventions to tailor cell fate during tissue regeneration.

These results have significant therapeutic implications. In the mouse model of colitis disclosed above, augmenting αKG levels ameliorates the deficit in secretory lineage cells associated with the disease and promotes tissue healing and regeneration.

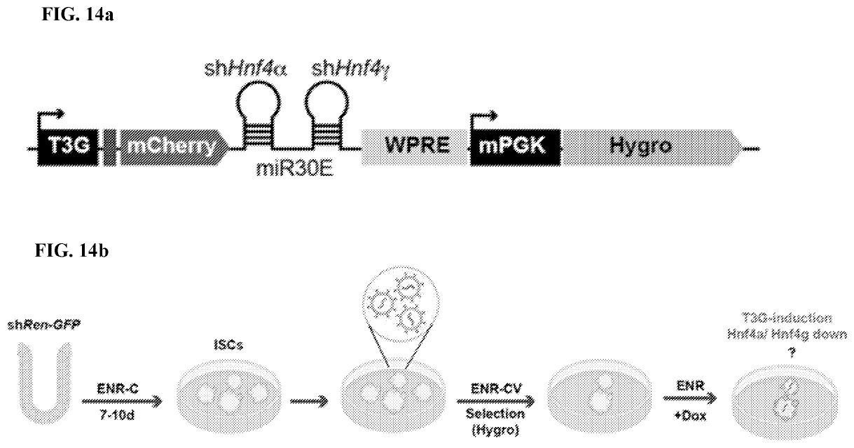

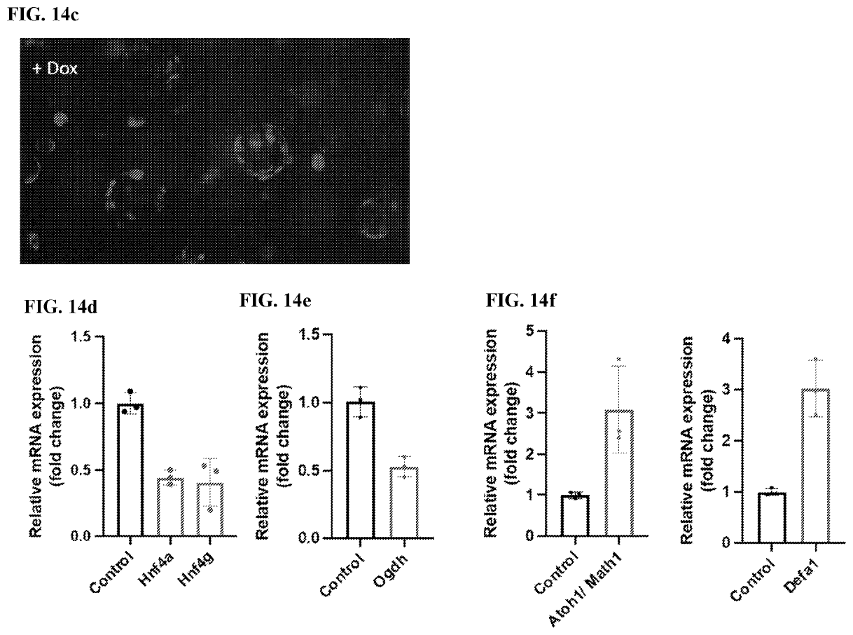

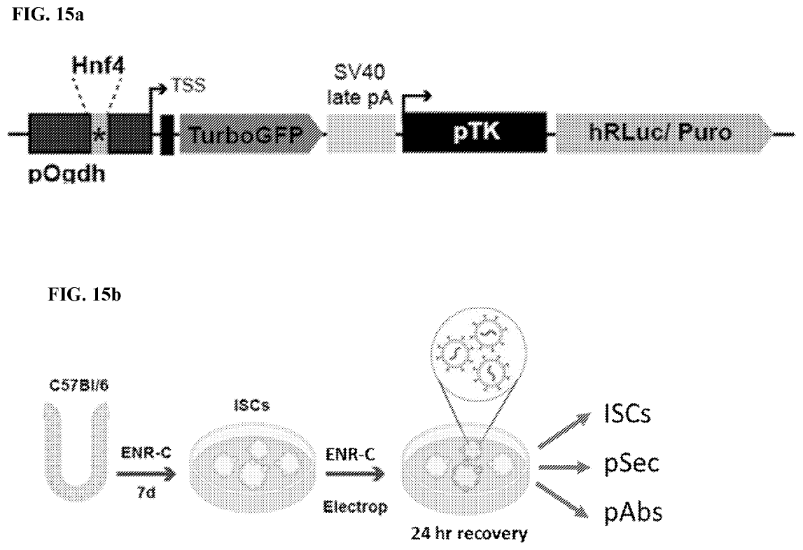

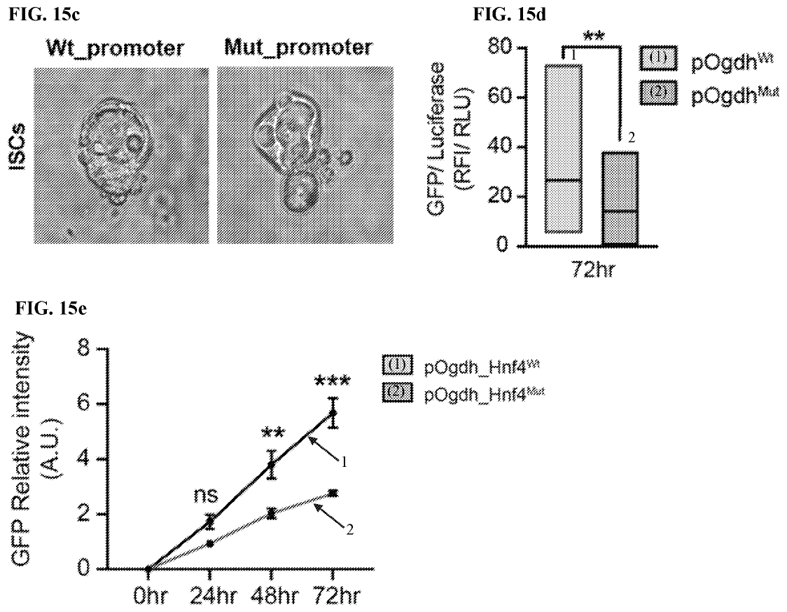

The following results provide strong biologic data (with mechanistic explanations) for how the TCA cycle can be used in different ways in different lineages and its therapeutic implications. Hnf4 modulates Ogdh expression and maintain enterocytic lineage (FIGS. 14-15).

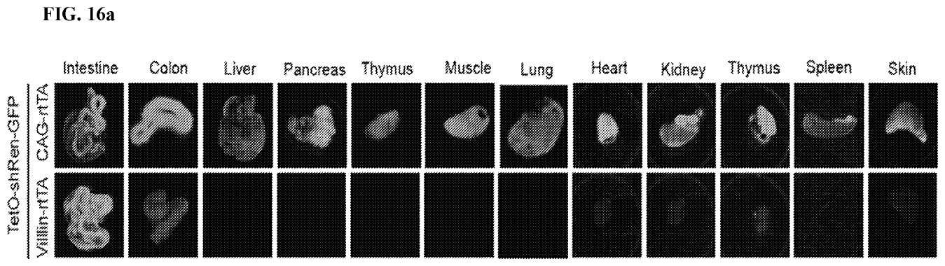

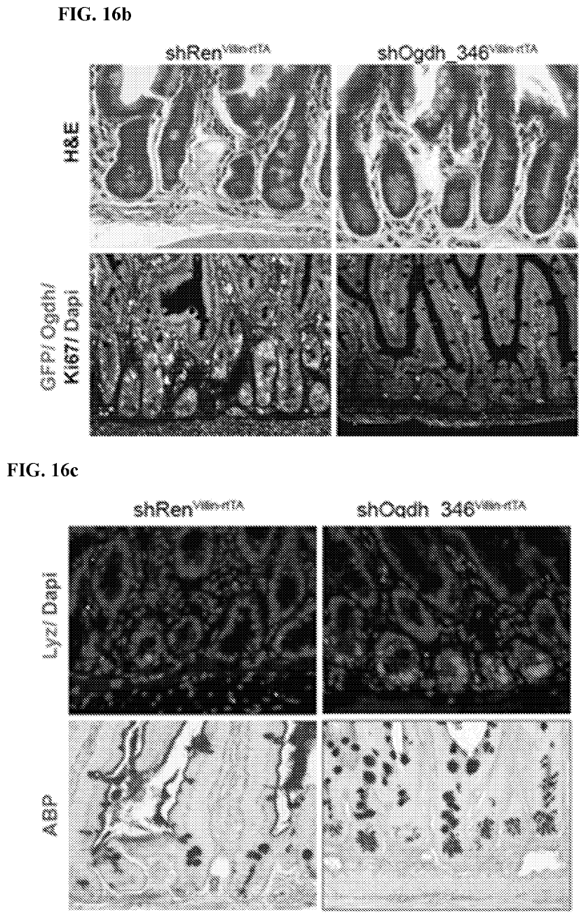

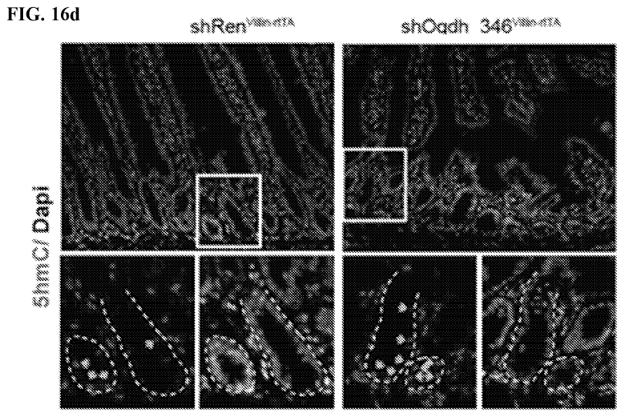

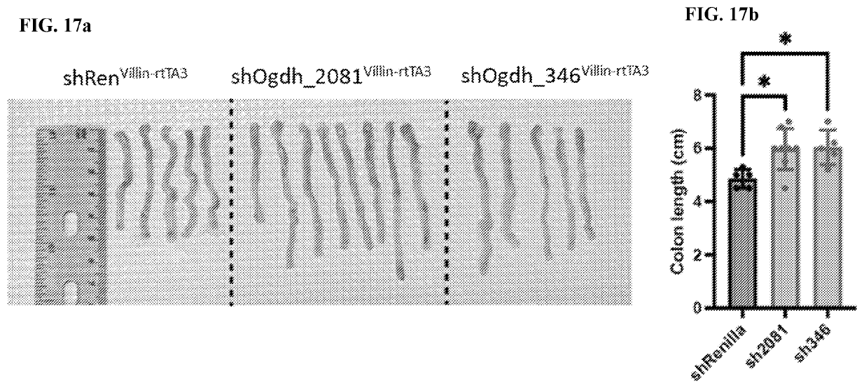

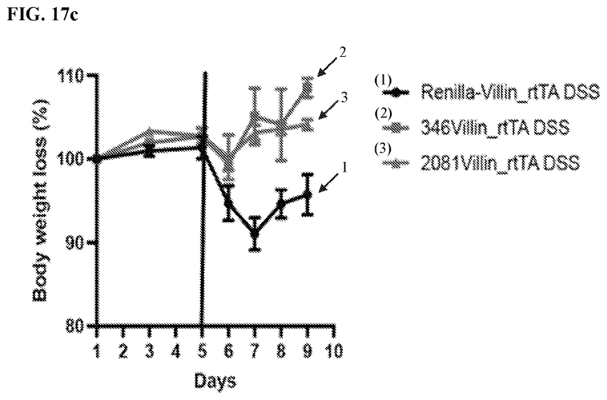

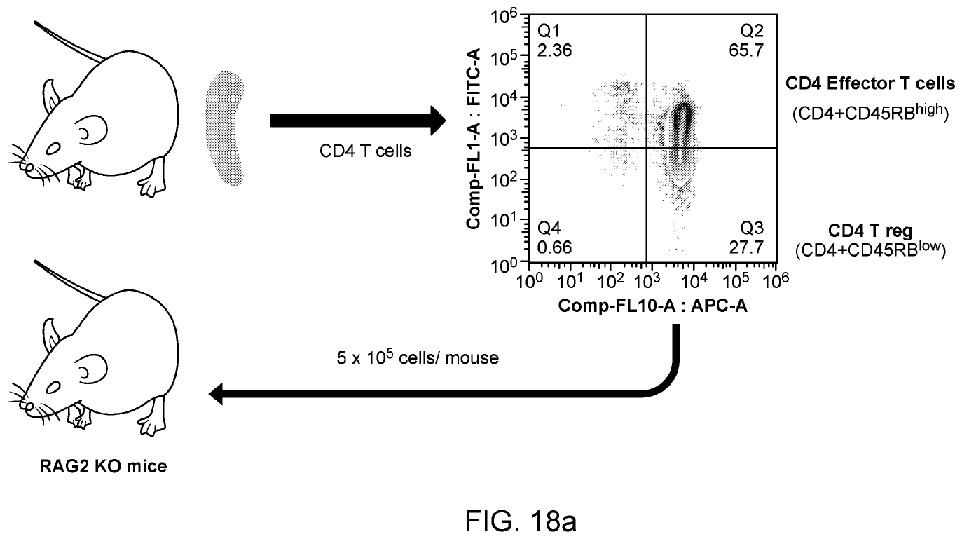

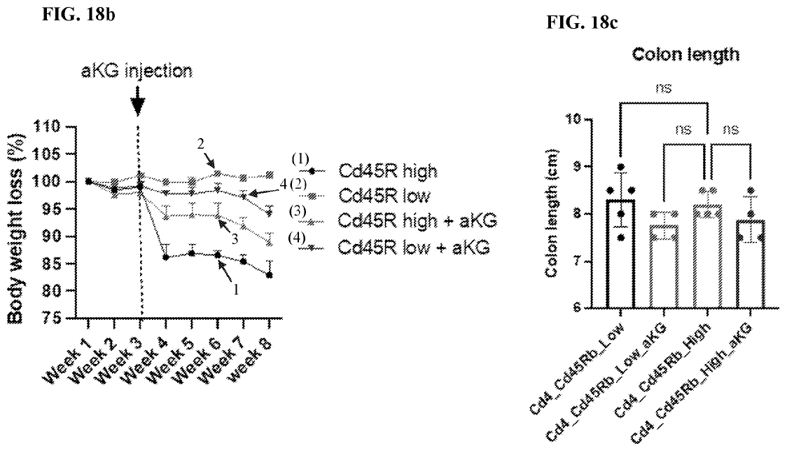

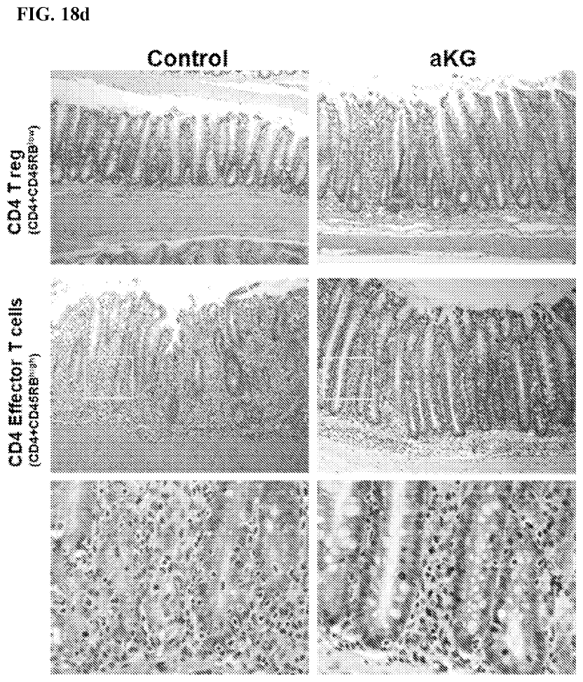

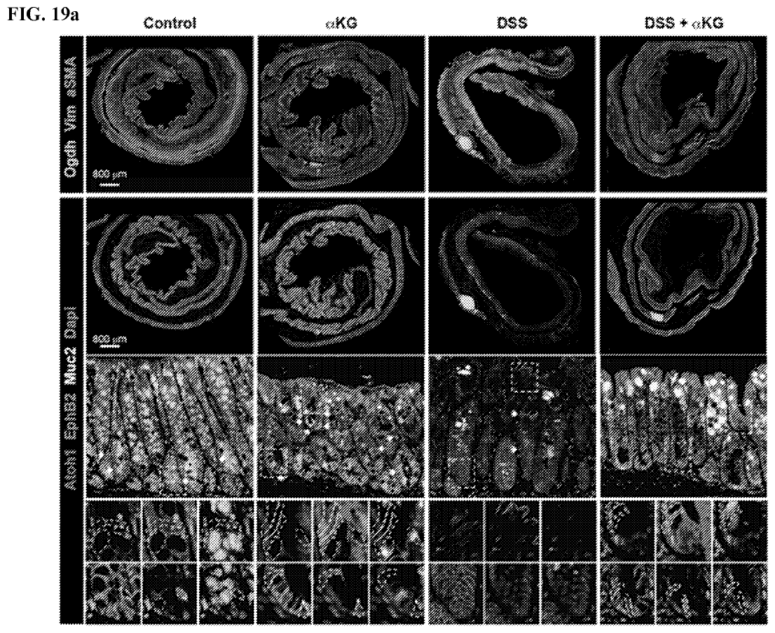

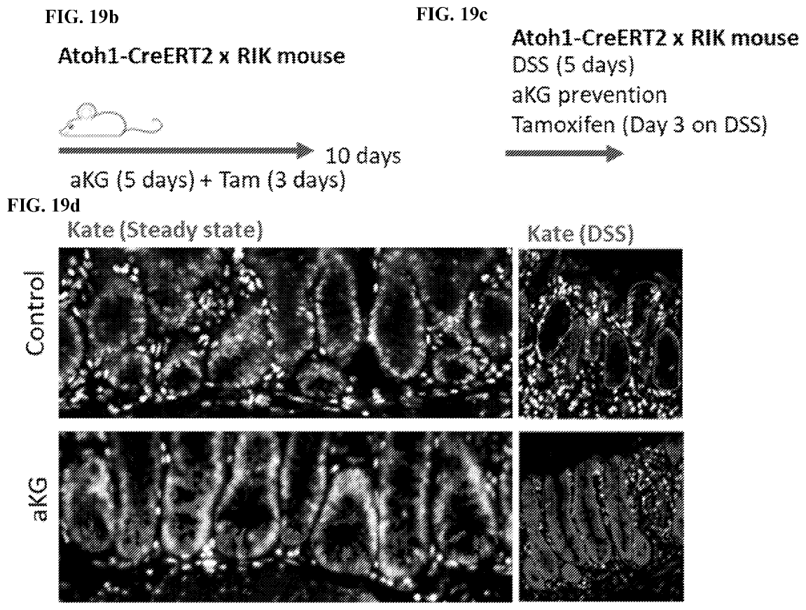

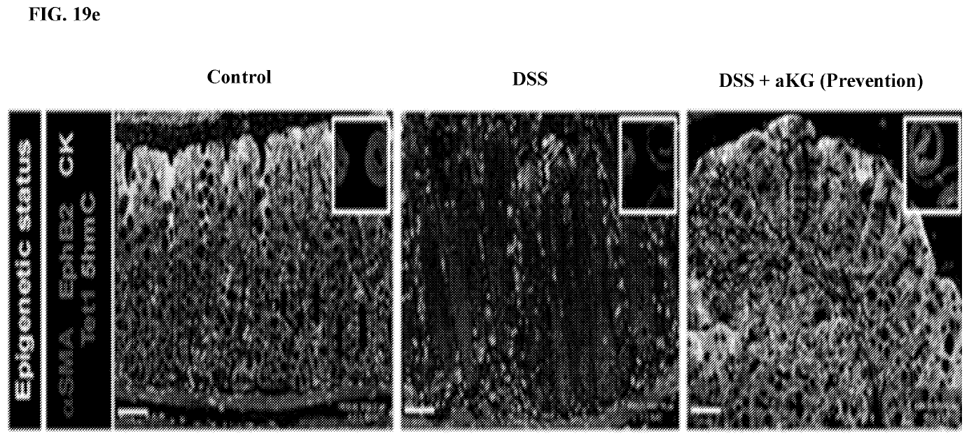

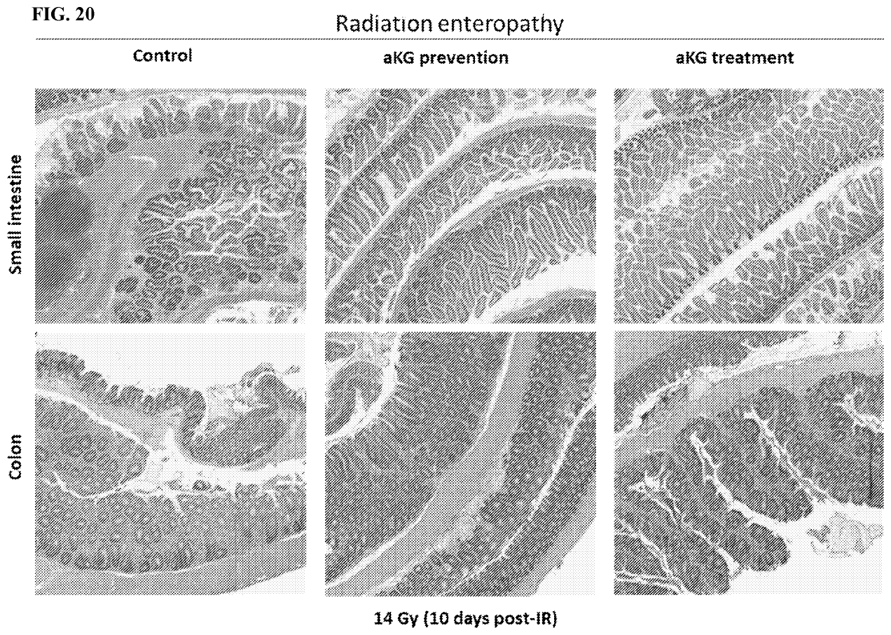

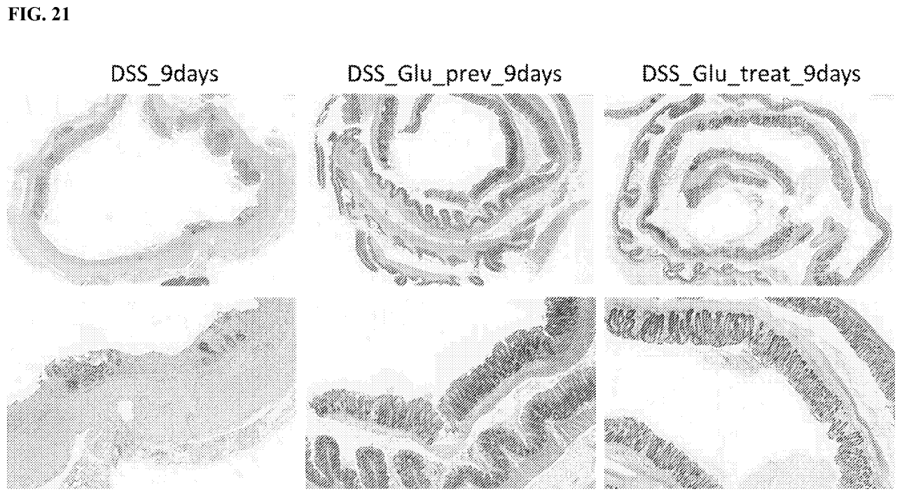

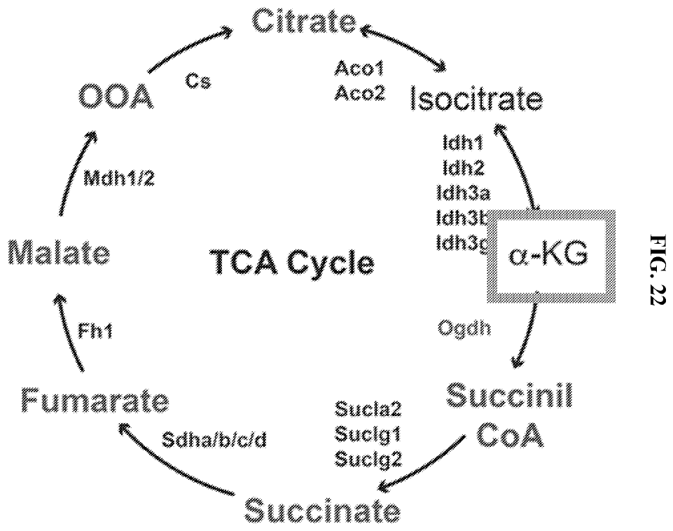

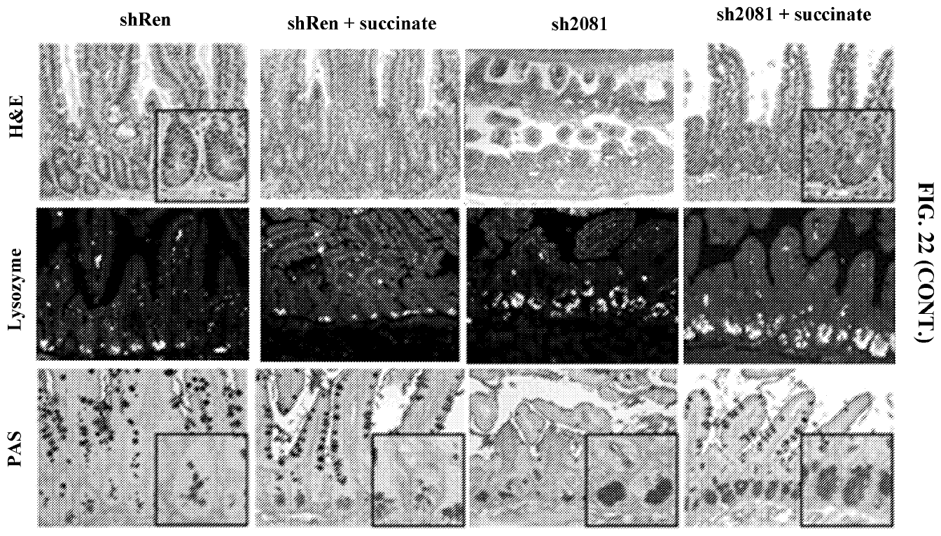

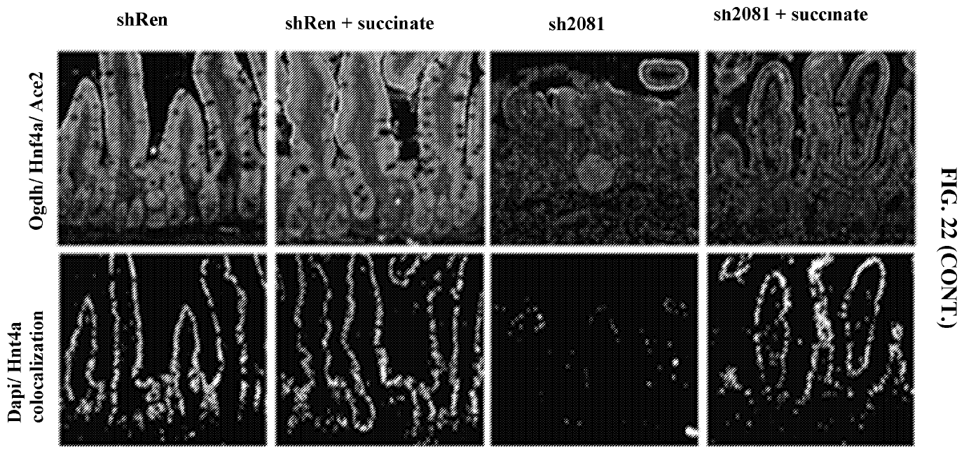

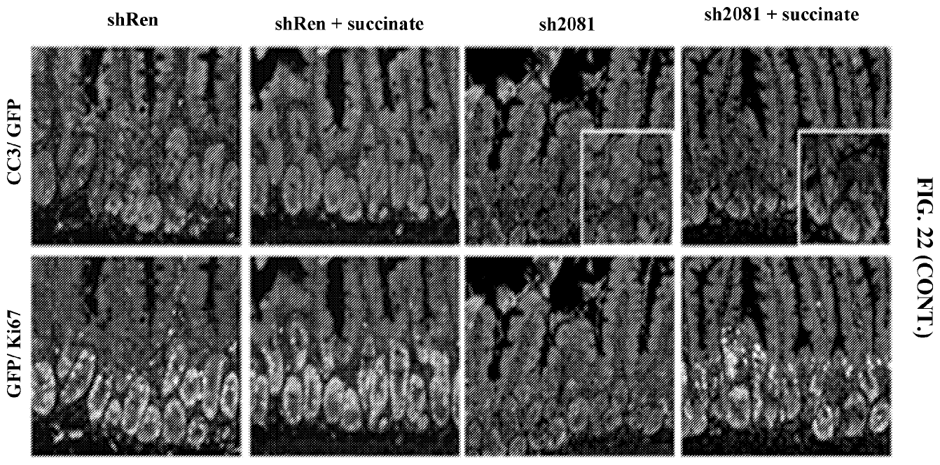

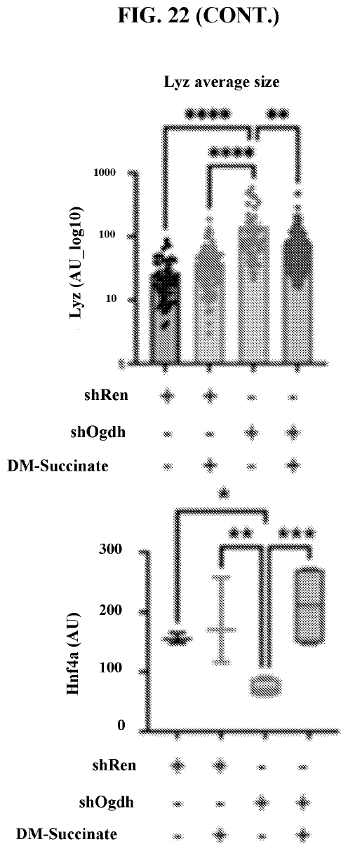

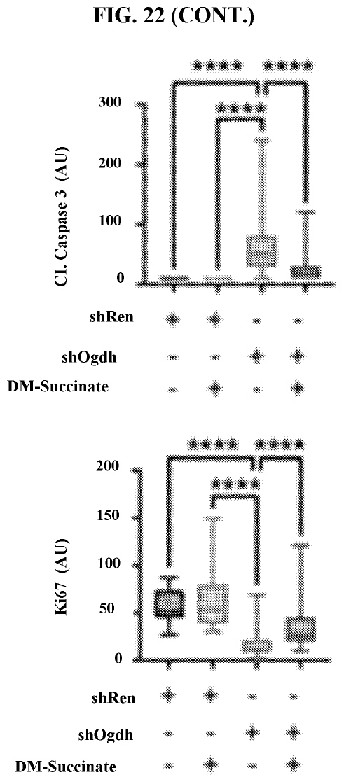

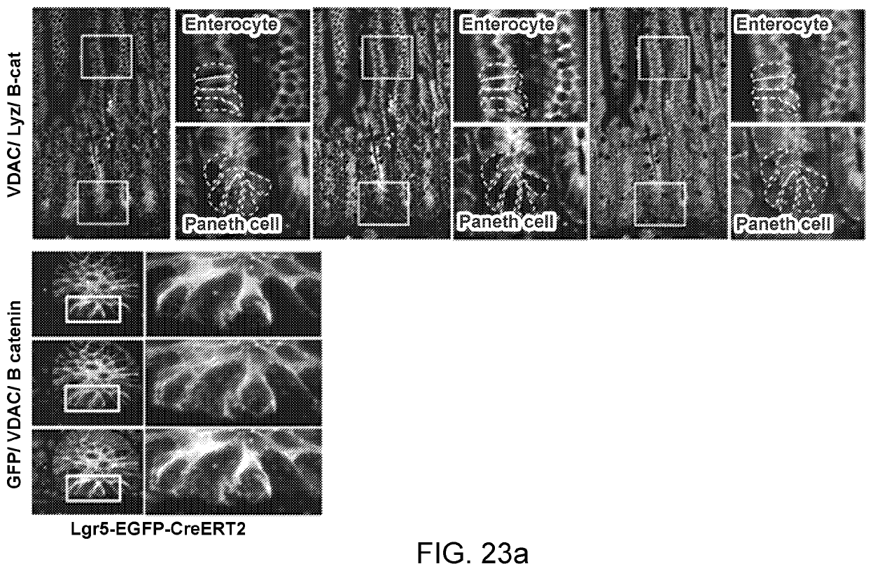

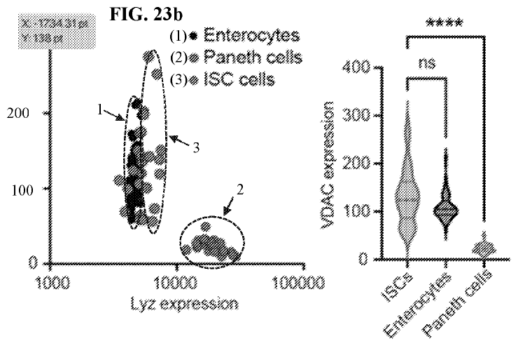

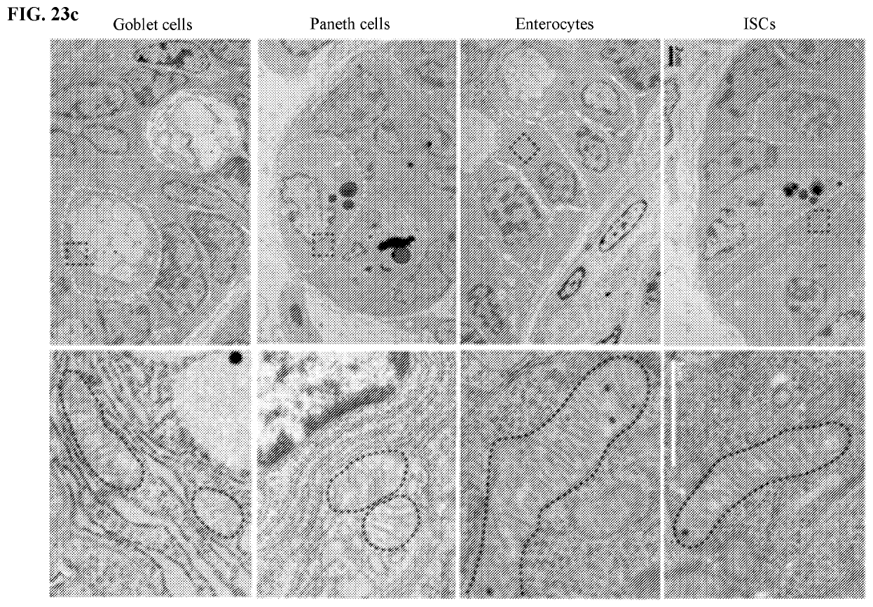

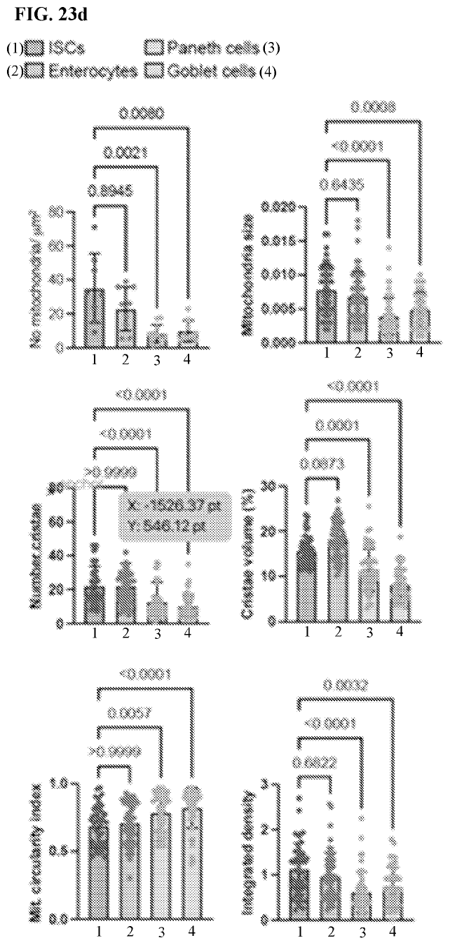

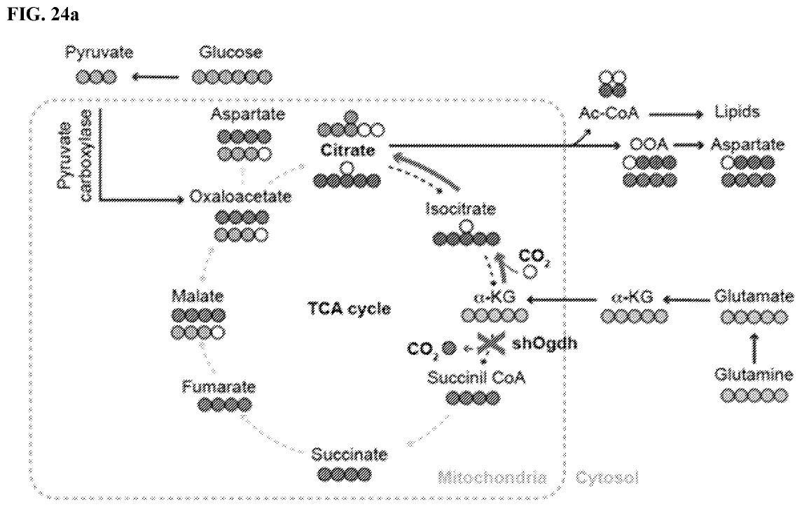

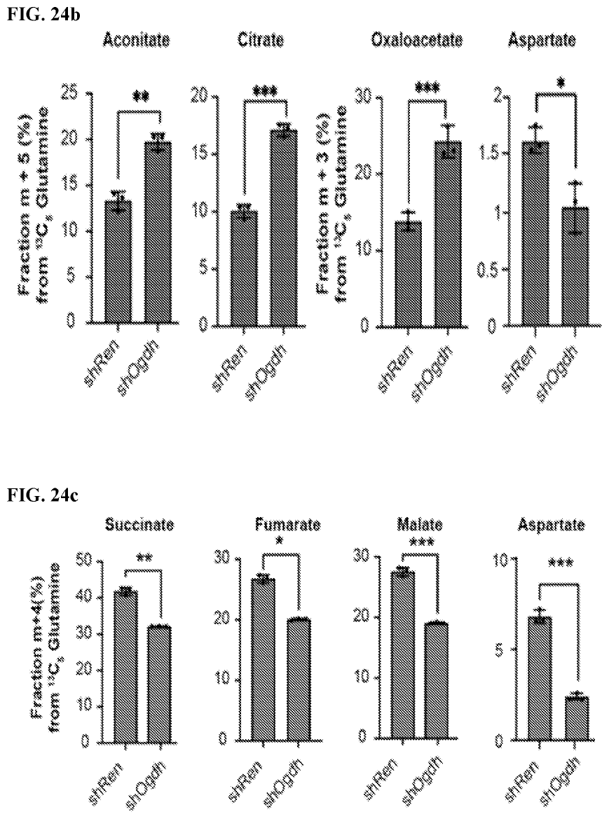

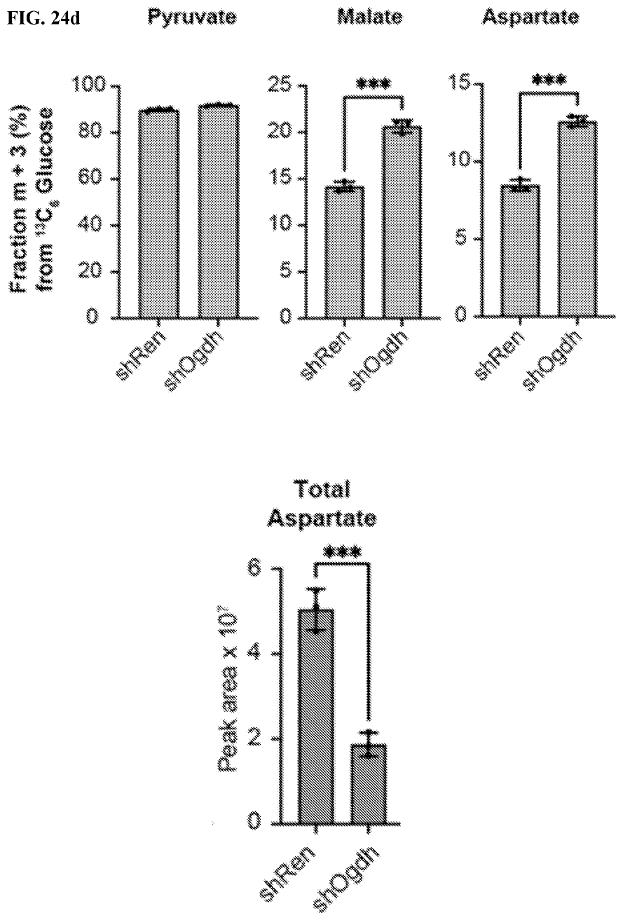

OGDH being important for biosynthesis functions in the enterocyte lineage (mirroring AML), while its inhibition leads to αKG increases driving differentiation of secretory lineage (FIG. 16). For colitis, the data show: 1) OGHD inhibition or αKG supplementation can stimulate intestinal regeneration of the secretory lineage in two model of colitis (FIGS. 17-18); 2) that succinate supplementation can rescue the negative effects of ODGH inhibition on the enterocyte lineage, while maintaining the positive benefits on this on promoting the secretory lineage (FIG. 22).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1 Morris, J. P. t. et al. alpha-Ketoglutarate links p53 to cell fate during tumour suppression. Nature 573, 595-599 (2019).
2 Carey, B. W., Finley, L. W., Cross, J. R., Allis, C. D. & Thompson, C. B. Intracellular alpha-ketoglutarate maintains the pluripotency of embryonic stem cells. Nature 518, 413-416 (2015).
3 Tran, T. Q. et al. alpha-Ketoglutarate attenuates Wnt signaling and drives differentiation in colorectal cancer. Nat Cancer 1, 345-358 (2020).
4 Gersemann, M. et al. Differences in goblet cell differentiation between Crohn's disease and ulcerative colitis. Differentiation 77, 84-94 (2009).
5 Wehkamp, J. & Stange, E. F. An Update Review on the Paneth Cell as Key to Ileal Crohn's Disease. Front Immunol 11, 646 (2020).
6 Scoville, E. A. et al. Alterations in Lipid, Amino Acid, and Energy Metabolism Distinguish Crohn's Disease from Ulcerative Colitis and Control Subjects by Serum Metabolomic Profiling. Metabolomics 14, 17 (2018).
7 Khaloian, S. et al. Mitochondrial impairment drives intestinal stem cell transition into dysfunctional Paneth cells predicting Crohn's disease recurrence. Gut 69, 1939-1951 (2020).
8 Connors, J., Dawe, N. & Van Limbergen, J. The Role of Succinate in the Regulation of Intestinal Inflammation. Nutrients 11 (2018).
9 Gehart, H. & Clevers, H. Tales from the crypt: new insights into intestinal stem cells. Nat Rev Gastroenterol Hepatol 16, 19-34 (2019).
10 Clevers, H. & Batlle, E. SnapShot: the intestinal crypt. Cell 152, 1198-1198 e1192 (2013).
11 Sato, T. et al. Single Lgr5 stem cells build crypt-villus structures in vitro without a mesenchymal niche. Nature 459, 262-265 (2009).
12 Chaves-Perez, A. et al. Transit-amplifying cells control R-spondins in the mouse crypt to modulate intestinal stem cell proliferation. J Exp Med 219 (2022).
13 McCarthy, N., Kraiczy, J. & Shivdasani, R. A. Cellular and molecular architecture of the intestinal stem cell niche. Nat Cell Biol 22, 1033-1041 (2020).
14 Al-Dewachi, H. S., Appleton, D. R., Watson, A. J. & Wright, N. A. Variation in the cell cycle time in the crypts of Lieberkuhn of the mouse. Virchows Arch B Cell Pathol Incl Mol Pathol 31, 37-44 (1979).
15 Cook, G. W. et al. Structural variation and its potential impact on genome instability: Novel discoveries in the EGFR landscape by long-read sequencing. PLoS One 15, e0226340 (2020).
16 Helander, H. F. & Fandriks, L. Surface area of the digestive tract—revisited. Scand J Gastroenterol 49, 681-689 (2014).
17 Parikh, K. et al. Colonic epithelial cell diversity in health and inflammatory bowel disease. Nature 567, 49-55 (2019).
18 Cliff, T. S. & Dalton, S. Metabolic switching and cell fate decisions: implications for pluripotency, reprogramming and development. Curr Opin Genet Dev 46, 44-49 (2017).
19 Rath, E. & Haller, D. Intestinal epithelial cell metabolism at the interface of microbial dysbiosis and tissue injury. Mucosal Immunol 15, 595-604 (2022).
20 Tong, W. et al. The intestine is a major contributor to circulating succinate in mice. FASEB J 36, e22546 (2022).
21 Beaumont, M. & Blachier, F. Amino Acids in Intestinal Physiology and Health. Adv Exp Med Biol 1265, 1-20 (2020).
22 Arnold, P. K. et al. A non-canonical tricarboxylic acid cycle underlies cellular identity. Nature 603, 477-481 (2022).
23 Baksh, S. C. & Finley, L. W. S. Metabolic Coordination of Cell Fate by alpha-Ketoglutarate-Dependent Dioxygenases. Trends Cell Biol 31, 24-36 (2021).
24 Haber, A. L. et al. A single-cell survey of the small intestinal epithelium. Nature 551, 333-339 (2017).
25 Biton, M. et al. T Helper Cell Cytokines Modulate Intestinal Stem Cell Renewal and Differentiation. Cell 175, 1307-1320 e1322 (2018).
26 Burclaff, J. et al. A Proximal-to-Distal Survey of Healthy Adult Human Small Intestine and Colon Epithelium by Single-Cell Transcriptomics. Cel/Mol Gastroenterol Hepatol 13, 1554-1589 (2022).
27 Barker, N. et al. Identification of stem cells in small intestine and colon by marker gene Lgr5. Nature 449, 1003-1007 (2007).
28 Yin, X. et al. Niche-independent high-purity cultures of Lgr5+ intestinal stem cells and their progeny. Nat Methods 11, 106-112 (2014).
29 Premsrirut, P. K. et al. A rapid and scalable system for studying gene function in mice using conditional RNA interference. Cell 145, 145-158 (2011).
30 Bolden, J. E. et al. Inducible in vivo silencing of Brd4 identifies potential toxicities of sustained BET protein inhibition. Cell Rep 8, 1919-1929 (2014).
31 Li, X. et al. A preclinical platform for assessing antitumor effects and systemic toxicities of cancer drug targets. Proc Natl Acad Sci USA 119, e2110557119 (2022).
32 Dow, L. E. et al. A pipeline for the generation of shRNA transgenic mice. Nat Protoc 7, 374-393 (2012).
33 Rodriguez-Colman, M. J. et al. Interplay between metabolic identities in the intestinal crypt supports stem cell function. Nature 543, 424-427 (2017).
34 Allen, E. L. et al. Differential Aspartate Usage Identifies a Subset of Cancer Cells Particularly Dependent on OGDH. Cell Rep 17, 876-890 (2016).
35 Hardie, D. G. AMP-activated protein kinase: an energy sensor that regulates all aspects of cell function. Genes Dev 25, 1895-1908 (2011).
36 Hardie, D. G. Minireview: the AMP-activated protein kinase cascade: the key sensor of cellular energy status. Endocrinology 144, 5179-5183 (2003).
37 Shaw, R. J. et al. The tumor suppressor LKB1 kinase directly activates AMP-activated kinase and regulates apoptosis in response to energy stress. Proc Natl Acad Sci USA 101, 3329-3335 (2004).
38 Chen, L. et al. A reinforcing HNF4-SMAD4 feed-forward module stabilizes enterocyte identity. Nat Genet 51, 777-785 (2019).
39 Smillie, C. S. et al. Intra- and Inter-cellular Rewiring of the Human Colon during Ulcerative Colitis. Cell 178, 714-730 e722 (2019).
40 Gassler, N. Paneth cells in intestinal physiology and pathophysiology. World J Gastrointest Pathophysiol 8, 150-160 (2017).
41 Fan, Z. et al. Generation of an oxoglutarate dehydrogenase knockout rat model and the effect of a high-fat diet. RSC Adv 8, 16636-16644 (2018).
42 Chassaing, B., Aitken, J. D., Malleshappa, M. & Vijay-Kumar, M. Dextran sulfate sodium (DSS)-induced colitis in mice. Curr Protoc Immunol 104, 15 25 11-15 25 14 (2014).
43 Bolger, A. M., Lohse, M. & Usadel, B. Trimmomatic: a flexible trimmer for Illumina sequence data. Bioinformatics 30, 2114-2120 (2014).
44 Dobin, A. et al. STAR: ultrafast universal RNA-seq aligner. Bioinformatics 29, 15-21 (2013).
45 Liao, Y., Smyth, G. K. & Shi, W. featureCounts: an efficient general purpose program for assigning sequence reads to genomic features. Bioinformatics 30, 923-930 (2014).
46 Love, M. I., Huber, W. & Anders, S. Moderated estimation of fold change and dispersion for RNA-seq data with DESeq2. Genome Biol 15, 550 (2014).
47 Chen, E. Y. et al. Enrichr: interactive and collaborative HTML5 gene list enrichment analysis tool. BMC Bioinformatics 14, 128 (2013).
48 Barriga, F. M. et al. MACHETE identifies interferon-encompassing chromosome 9p21.3 deletions as mediators of immune evasion and metastasis. Nat Cancer 3, 1367-1385 (2022).
49 Ludikhuize, M. C., Meerlo, M., Burgering, B. M. T. & Rodriguez Colman, M. J. Protocol to profile the bioenergetics of organoids using Seahorse. STAR Protoc 2, 100386 (2021).
50 Weigmann, B. et al. Isolation and subsequent analysis of murine lamina propria mononuclear cells from colonic tissue. Nat Protoc 2, 2307-2311 (2007).
51 Burdziak, C. et al. Epigenetic plasticity cooperates with cell-cell interactions to direct pancreatic tumorigenesis. Science 380, eadd5327 (2023).
52 Moffitt, J. R. et al. Molecular, spatial, and functional single-cell profiling of the hypothalamic preoptic region. Science 362 (2018).
53 Moffitt, J. R. et al. High-throughput single-cell gene-expression profiling with multiplexed error-robust fluorescence in situ hybridization. Proc Natl Acad Sci USA 113, 11046-11051 (2016).

1. A method for treating or preventing a gastrointestinal inflammatory disease in a subject in need thereof comprising administering to the subject a therapeutically effective amount of an agent that increases alpha-ketoglutarate (αKG) expression and/or activity.

2. A method for selecting a subject suffering from a gastrointestinal inflammatory disease for treatment with an agent that increases alpha-ketoglutarate (αKG) activity comprising
a. detecting OGDH mRNA and/or OGDH polypeptide levels in a biological sample obtained from the patient that are elevated relative to a predetermined threshold or a reference sample obtained from a healthy control subject; and
b. administering to the subject an effective amount of an agent that increases αKG activity.

3. The method of claim 1, wherein the gastrointestinal inflammatory disease is colitis or Chron's disease.

4. The method of claim 1, wherein the agent is αKG, glutamine, succinate, or dimethyl-succinate; or wherein the agent inhibits expression and/or activity of a component of oxoglutarate dehydrogenase (OGDH) complex.

5. (canceled)

6. The method of claim 4, wherein the component of OGDH complex is oxoglutarate dehydrogenase (OGDH), dihydrolipoyl succinyltransferase (DLST), or dihydrolipoyl dehydrogenase (DLD).

7. The method of claim 4, wherein the agent is a small molecule, an inhibitory nucleic acid that specifically targets the component of OGDH complex, or a neutralizing antibody that specifically targets the component of OGDH complex.

8. The method of claim 7, wherein the small molecule inhibits OGDH and is succinyl phosphonate, (S)-2-[(2,6-dichlorobenzoyl)amino]succinic acid (AA6), KGD09, KGD02, or an azide-alkyne cyclized derivative of KGD09 or KGD02; or
wherein the small molecule inhibits DLST and is obatoclax mesylate, CP724714, LY317615 (enzastaurin), gemcitabine, BMS 708163 (Avagacestat), Lapatinib, or IPA3; or
wherein the small molecule inhibits DLD and is 5-methoxyindole-2-carboxylic acid (MICA) or a valproic acid (VPA) metabolite (e.g., valproyl-CoA, valproyl-dephospho-CoA); or
wherein the inhibitory nucleic acid that specifically targets the component of OGDH complex is a siRNA, a shRNA, an antisense oligonucleotide, or a sgRNA.

9. (canceled)

10. (canceled)

11. (canceled)

12. The method of claim 8, wherein the inhibitory nucleic acid that specifically targets the component of OGDH complex is operably linked to a heterologous promoter, an inducible promoter, a constitutive promoter, a tissue-specific promoter, or an ubiquitous promoter.

13. The method of claim 12, wherein the inhibitory nucleic acid that specifically targets the component of OGDH complex is selectively expressed in intestinal epithelial cells.

14. The method of claim 1, wherein the agent is administered orally, topically, intranasally, systemically, intravenously, subcutaneously, intraperitoneally, intradermally, intraocularly, iontophoretically, transmucosally, or intramuscularly.

15. The method of claim 1, further comprising separately, sequentially or simultaneously administering one or more additional therapeutic agents to the subject, wherein the one or more additional therapeutic agents are selected from among aminosalicylates (e.g., sulfasalazine, mesalamine, olsalazine, balsalazide), corticosteroids (e.g., Prednisone, Prednisolone, Methylprednisolone, Budesonide), azathioprine, 6-mercaptopurine, cyclosporine, tacrolimus, ozanimod, tofacitinib, upadacitinib, etrasimod, adalimumab, golimumab, infliximab, ustekinumab and vedolizumab.

16. The method of claim 1, wherein administration of the agent that increases αKG activity results in an increased abundance of secretory intestinal cells in the subject.

17. The method of claim 1, wherein administration of the agent does not alter biosynthetic function or viability of enterocytes in the subject.

18. The method of claim 1, wherein the agent that increases αKG activity is specifically targeted for delivery into intestinal epithelium using a nanocarrier.

19. The method of claim 18, wherein the nanocarrier comprises an extracellular vesicle, a lipid-based nanoparticle, a microsphere, a liposome, an inorganic nanoparticle, a SPION, a SiNP, a SNEDD, or a polymeric nanoparticle.

20. The method of claim 19, wherein the nanocarrier is functionalized with PEG, listeriolysin O (LLO), silica mesoporous microparticles, an antibody, a carbohydrate, or a ligand that binds a receptor that overexpressed in intestinal epithelium.

21. The method of claim 1, wherein the agent that increases αKG activity further comprises a pH-dependent release coating or a delayed-release coating.

22. The method of claim 21, wherein the pH-dependent release coating or the delayed-release coating comprises one or more of hyaluronic acid, alginate, Eudragit, polysaccharides, inulin, xanthan gum, locust bean gum, portulaca-derived polysaccharides, pectin, guar gum, carboxymethyl starch (CM), Layered double hydroxides (LDHs), calixarenes, and cholesteryl hemisuccinate (CHEMS).

23. A method for enhancing tissue regeneration in a subject that has received radiation therapy comprising administering to the subject a therapeutically effective amount of alpha-ketoglutarate (αKG) or glutamine.

24. The method of claim 23, wherein the radiation therapy is external beam radiation therapy: or wherein tissue regeneration comprises regeneration of intestinal tissue or colon tissue.

25. (canceled)
